(19) United States
(12) Patent Application Publication
(10) Pub. No.: US 2015/0051144 A1
(43) Pub. Date: Feb. 19, 2015

(54) COMPOUNDS FOR THE TREATMENT OF DYSLIPIDEMIA AND OTHER DISEASES

(71) Applicant: Cadila Healthcare Limited, Ahmedabad (IN)

(72) Inventors: Harikishore Pingali, Ahmedabad (IN); Vrajesh Pandya, Ahmedabad (IN)

(21) Appl. No.: 14/394,978

(22) PCT Filed: Sep. 26, 2012
(86) PCT No.: PCT/IN2012/000640

§ 371 Date: Oct. 16, 2014

(30) Foreign Application Priority Data
Jun. 25, 2012 (IN) 1814MUM2012

(51) Int. Cl. C07D 333/22; A61K 31/351; C07C 237/52; A61K 31/5375; A61K 31/496; C07D 211/60; A61K 31/445; A61K 31/5377; C07D 209/04; C07D 309/06; A61K 31/495

(52) U.S. Cl. CPC C07D 333/22; C07D 309/06; A61K 31/351; C07C 237/52; A61K 31/5375; A61K 31/495; C07D 211/60; A61K 31/445; A61K 31/5377; C07D 209/04; A61K 31/496
USPC 514/6.5; 549/426; 514/459; 544/168; 514/237.8; 544/162; 514/237.5; 544/391; 514/255.01; 546/221; 514/327; 544/146; 514/231.5; 544/373; 514/254.09; 514/7.2

(57) ABSTRACT

The present invention relates to compounds of the general formula (I), their tautomeric forms, their stereoisomers, their pharmaceutically acceptable salts, pharmaceutical compositions containing them, methods for their preparation, use of these compounds in medicine and the intermediates involved in their preparation. The present invention is directed towards compounds which can be used to treat diseases such as hyperlipidemia and also have a beneficial effect on cholesterol.

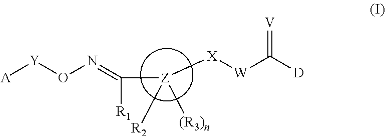

### FIELD OF INVENTION

The present invention relates to compounds of the general formula (I), their tautomeric forms, their stereoisomers, their pharmaceutically acceptable salts, pharmaceutical compositions containing them, methods for their preparation, use of these compounds in medicine and the intermediates involved in their preparation.

The present invention is directed towards compounds which can be used to treat diseases such as hyperlipidemia and also have a beneficial effect on cholesterol.

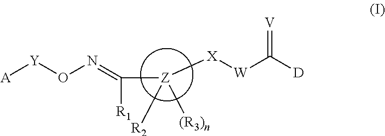

The compounds of the general formula (I) lower blood glucose, lower or modulate triglyceride levels and/or cholesterol levels and/or low-density lipoproteins (LDL) and raises the high-density lipoproteins (HDL) plasma levels and hence are useful in combating different medical conditions, where such lowering (and raising) is beneficial. Thus, it could be used in the treatment and/or prophylaxis of obesity, hyperlipidemia, hypercholesteremia, hypertension, atherosclerotic disease events, vascular restenosis, diabetes and many other related conditions.

The compounds of general formula (I) are useful to prevent or reduce the risk of developing atherosclerosis, which leads to diseases and conditions such as artereosclerotic cardiovascular diseases, stroke, coronary heart diseases, cerebrovascular diseases, peripheral vessel diseases and related disorders.

These compounds of general formula (I) are useful for the treatment and/or prophylaxis of metabolic disorders loosely defined as Syndrome X. The characteristic features of Syndrome X include initial insulin resistance followed by hyperinsulinemia, dyslipidemia and impaired glucose tolerance. The glucose intolerance can lead to non-insulin dependent diabetes mellitus (NIDDM, Type 2 diabetes), which is characterized by hyperglycemia, which if not controlled may lead to diabetic complications or metabolic disorders caused by insulin resistance. Diabetes is no longer considered to be associated only with glucose metabolism, but it affects anatomical and physiological parameters, the intensity of which vary depending upon stages/duration and severity of the diabetic state. The compounds of this invention are also useful in prevention, halting or slowing progression or reducing the risk of the above mentioned disorders along with the resulting secondary diseases such as cardiovascular diseases, like arteriosclerosis, atherosclerosis; diabetic retinopathy, diabetic neuropathy and renal disease including diabetic nephropathy, glomerulonephritis, glomerular sclerosis, nephrotic syndrome, hypertensive nephrosclerosis and end stage renal diseases, like microalbuminuria and albuminuria, which may be result of hyperglycemia or hyperinsulinemia.

The compounds of the present invention can be useful as aldose reductase inhibitors; for improving cognitive functions in dementia, and in the treatment and/or prophylaxis of disorders such as psoriasis, polycystic ovarian syndrome (PCOS), cancer, osteoporosis, leptin resistance, inflammation and inflammatory bowel diseases, wound healing, xanthoma, pancreatitis, myotonic dystrophy, endothelial cell dysfunction and hyperlipidemia.

### BACKGROUND OF THE INVENTION

Higher LDL cholesterol levels in the plasma increase cardiovascular risk and reduction in the levels of LDL would decrease CVD risk by a comparable percentage (PNAS, 2009, 106, 9546-9547). Clearance of LDL cholesterol from plasma is through the action of LDL receptors in the liver and LDL receptors are cell surface glycoproteins that bind to apoliporpotein B100 (apoB100) on LDL particles with high affinity and mediate their endocytic uptake (Journal of Biological Chemistry, 2009, 284, 10561-10570). Defect in hepatic cholesterol clearance and elevated levels of plasma LDL cholesterol that result from the mutations cause familial hypercholesterolemia. Such mutations are identified in the human LDL receptor and later in apolipoprotein-B (Nature Structural and Molecular Biology, 2007, 14, 413-419). Recently, mutations within the pro-protein convertase subtilisin/kexin of the subtype 9 (PCSK 9) gene were found to represent a third class of mutations associated with autosomal dominant hypercholesterolemia (ADH). Abifadel et al in 2003 discovered pro-protein convertase subtilisin/kexin of the subtype 9 as the third gene involved in autosomal dominant hypercholesterolaemia (ADH) (Nature Genetics, 2003, 34, 154-156, Trends in Biochemical Sciences, 2008, 33, 426-434). Several missense mutations (S127R, D129G, F216L, D374H, D374Y) are associated with hypercholesterolemia and premature atherosclerosis (J Lipid Res. 2008, 49, 1333-1343). Loss-of-function mutations (R46L, L253F, A433T) lead to elevated receptor abundance, enhancing clearance of LDL cholesterol from the circulation and reducing cardiovascular risk (Nature Structural and Molecular Biology, 2007, 14, 413-419).

Pro-protein convertase subtilisin/kexin of the subtype 9 belongs to the subtilisin family of serine proteases and its protein structure consists of a pro-domain, catalytic domain, and cysteine/histidine rich C-terminal domain (Structure, 2007, 15, 545-552). Unlike other pro-protein convertases, wherein the pro-domain is further proteolytically processed to activate the serine protease, the pro-domain of secreted subtype remains intact and tightly bound. Within endoplasmic reticulum this enzyme undergoes autocatalytic process which results in release of ˜14 kDa prodomain that remains associated with the catalytic/C-terminal domains; wherein the pro-domain serves as both a folding chaperon and as an inhibitor of enzymatic activity (Journal of Biological Chemistry, 2009, 284, 10561-10570).

It is well documented that epidermal growth factor-like repeat A (EGF-A) of LDLR interacts with this pro-protein subtype mainly with residues 367-3.81. This EGF-A interaction site is located >20 Å from the catalytic site of this pro-protein subtype. Once EGF-A and this pro-protein subtype interacts they form a complex with the LDLR that enters endosomal pathway and hence LDLR recycling is prevented leading to LDLR degradation. Detailed molecular mechanisms explaining the association of LDLR and this pro-protein subtype and LDLR degradation is not very clear (Drug News Perspectives, 2008, 21, 323-330). Because of inhibition of LDLR recycling, number of LDL receptors on the cell surface are decreased and this increases plasma LDL levels (PNAS, 2009, 106, 9546-9547).

Various approaches for inhibiting this pro-protein subtype are reported, including gene silencing by siRNA or antisense oligonucleotides, mAb disrupting protein-protein interactions or by peptides; all the above-mentioned strategies have shown lowering of LDL cholesterol which may be effective therapy for treating hypercholesterolemia (Biochemical Journal, 2009, 419, 577-584; PNAS, 2008, 105, 11915-11920; Journal of Lipid Research, 2007, 48, 763-767; PNAS, 2009, 106, 9820-9825). However, very little success has been reported in trying to inhibit this pro-protein subtype by using small molecules. Small molecule inhibitors of this pro-protein subtype has its obvious clinical and therapeutic benefit over the other approaches as discussed above for the inhibition of pro-protein convertase subtilisin/kexin of the subtype 9. Small molecule inhibitors of this subtype have been disclosed by us in our application nos. 3556/MUM/2010 & 2292/MUM/2009. We herein disclose novel small molecules which have shown to inhibit the pro-protein convertase subtilisin/kexin of the subtype 9 in in-vitro studies and therefore provides an alternate beneficial approach for treating patients in need of such therapy.

### Preferred Embodiments of the Invention

An important object of the present invention is to provide novel substituted oximino derivatives represented by the general formula (I), their tautomeric forms, their stereoisomers, their pharmaceutically acceptable salts, and pharmaceutical compositions containing them or their mixtures thereof.

In an embodiment of the present invention is provided a process for the preparation of novel substituted oximino derivatives and their derivatives represented by the general formula (I), their tautomeric forms, their stereoisomers, their pharmaceutically acceptable salts.

In a further embodiment of the present invention is provided pharmaceutical compositions containing compounds of the general formula (I), their tautomeric forms, their stereoisomers, their pharmaceutically acceptable salts, or their mixtures in combination with suitable carriers, solvents, diluents and other media normally employed in preparing such compositions.

In a further embodiment of the present invention is provided process for treatment of diseases such as dyslipidemia, hyperlipidemia etc. by providing therapeutically effective amount of the compounds of formula (I) or their pharmaceutically acceptable salts or their suitable pharmaceutical compositions.

The above and other embodiments are described in details hereinafter.

### DETAILED DESCRIPTION OF THE INVENTION

Accordingly, the present invention relates to compounds of the general formula (I),

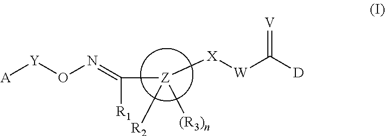

‘A’ represents an optionally substituted single or fused or spirocyclic or bridgeheaded group selected from aryl, heterocyclyl or cycloalkyl groups;

In a preferred embodiment, ‘A’ is selected from optionally substituted aryl or heterocyclyl groups;

In a further preferred embodiment, when ‘A’ represents and aryl group, the aryl group may be selected from substituted or unsubstituted monocyclic or bicyclic aromatic groups;

In a still further preferred embodiment, the aryl group is an optionally substituted phenyl group.

In an embodiment, when ‘A’ represents a heterocyclyl group, the heterocyclyl group may be selected from single or fused mono, bi or tricyclic aromatic or non-aromatic groups containing one or more hetero atoms selected from O, N or S(O)ₒ wherein ‘o’ represents integers from 0 to 2;

In a preferred embodiment, the heterocyclyl group may be selected from pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzofuranyl, benzothienyl, indolinyl, indolyl, azaindolyl, azaindolinyl, pyrazolopyrimidinyl, azaquinazolinyl, pyridofuranyl, pyridothienyl, thienopyrimidyl, quinolinyl, pyrimidinyl, pyrazolyl, quinazolinyl, pyridazinyl, triazinyl, benzimidazolyl, benzotriazolyl, phthalazynil, naphthylidinyl, purinyl, carbazolyl, phenothiazinyl, phenoxazinyl, benzoxazolyl, benzothiazolyl, thiazepinyl, oxazolidinyl, thiazolidinyl, dihydrothiophene, dihydropyran, dihydrofuran, dihydrothiazole, benzopyranyl, benzopyranonyl, benzodihydrofuranyl, benzodihydrothienyl, pyrazolopyrimidonyl, azaquinazolinoyl, thienopyrimidonyl, quinazolonyl, pyrimidonyl, benzoxazinyl, benzoxazinonyl, benzothiazinyl, benzothiazinonyl, thieno piperidinyl and the like;

‘Y’ represents either a bond or substituted or unsubstituted linear or branched (C₁-C₆)alkyl, (C₂-C₆)alkenyl groups or the groups represented by ‘-U(CH₂)ₘ—’ wherein U represents O, S(O)ₒ, NR₈; ‘m’ represents integers from 2 to 4 and R₈ represents H, substituted or unsubstituted linear or branched (C₁-C₆)alkyl;

‘V’ represents O or S;

‘Z’ represents an optionally substituted single or fused group selected from aryl, heterocyclyl or cycloalkyl groups;

In a preferred embodiment, ‘Z’ is selected from optionally substituted aryl or heterocyclyl groups;

In a further preferred embodiment, when ‘Z’ represents an aryl group, the aryl group may be selected from substituted or unsubstituted monocyclic or bicyclic aromatic groups;

In a still further preferred embodiment, the aryl group is an optionally substituted phenyl group.

When ‘Z’ represents a heterocyclyl group, the heterocyclyl group may be selected from single or fused mono or bi cyclic aromatic or non-aromatic groups containing one or more hetero atoms selected from O, N or S(O)ₒ;

In a still preferred embodiment, when ‘Z’ represents heteroaromatic group, the heteroaromatic group may be selected from pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzofuranyl, benzothienyl, indolinyl, indolyl, azaindolyl, azaindolinyl, pyrazolopyrimidinyl, azaquinazolinyl, pyridofuranyl, pyridothienyl, thienopyrimidyl, quinolinyl, pyrimidinyl, pyrazolyl, quinazolinyl, pyridazinyl, triazinyl, benzimidazolyl, benzotriazolyl, phthalazynil, naphthylidinyl, purinyl, carbazolyl, phenothiazinyl, phenoxazinyl, benzoxazolyl, benzothiazolyl groups.

‘X’ represents either a bond, or may be selected from O, S(O)ₒ or NR₈; wherein R₈ is as defined earlier;

‘W’ represents substituted or unsubstituted linear or branched (C₁-C₆)alkyl, (C₂-C₆)alkenyl groups;

R₁ represents hydrogen, optionally substituted, (C₁-C₆)alkyl, (C₃-C₆)cycloalkyl, alkoxyalkyl, hydroxyalkyl, aminoalkyl, aryl, heterocyclyl, aralkyl, heterocyclylalkyl groups;

R₂ represents hydrogen, or the groups selected from (C₁-C₆)alkyl, aryl, heterocyclyl, aralkyl, heterocyclylalkyl, (C₁-C₆)alkoxy, hydroxyalkyl, thio(C₁-C₆)alkyl, amino, aminoalkyl, alkylamino, halogen, hydroxyl, ester, formyl, acyl, haloalkyl each of which may be optionally substituted;

Alternatively R₁ and R₂ wherever possible, together may form 4 to 7 membered saturated or partially saturated ring containing from 0-2 additional heteroatoms selected from the group consisting of N, O, and S(O)ₒ;

R₃ at each occurrence independently represents hydrogen, halogen, (C₁-C₃)alkyl, halo(C₁-C₃)alkyl, (C₁-C₃)alkoxy, hydroxyl, ester, formyl, acyl, thio(C₁-C₃)alkyl, sulfenyl derivatives, sulfonyl derivatives;

‘D’ represents —NR₄R₅ or the group —N(R₆)(CH₂)ₚCONR₇R₈; optionally substituted spirocyclic or bridgeheaded group containing from 0-2 additional heteroatoms selected from the group consisting of N, O, and S(O)ₒ;

R₄, R₅, R₆ at each occurrence independently represents H, (C₁-C₆) linear or branched alkyl, (C₁-C₆) linear or branched alkenyl, (C₁-C₆) linear or branched alkynyl, hydroxy, (C₁-C₆) alkoxy, (C₁-C₆) alkenoxy, hydroxy(C₁-C₆)alkyl, alkoxyalkyl, haloalkyl, (C₃-C₆) cycloalkyl, thio(C₁-C₆)alkyl, (C₁-C₆)alkylthio, halo, oxo, imino, nitro, aryl, heterocyclyl, optionally substituted amino, amino(C₁-C₆)alkyl, alkylamino, cyano, formyl, acyl, haloalkoxy, aryl, aryloxy, aralkyl, aralkoxy, heterocyclyl, heterocyclylalkyl, heterocycloxy, heterocyclylalkoxy groups or the groups selected from carboxylic acid and its derivatives such as esters and amides, alkylsulfonyl, alkylsulfonylamino, alkylsulfonyloxy, each of which may be optionally substituted with a provisio that when ‘D’ represents —NR₄R₅ and either of R₄ or R₅ is H the other one does not represent

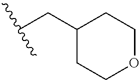 [SMILES: *CC1CCOCC1]

In an alternate embodiment, R₄ and R₅ wherever possible, together may form optionally substituted 4 to 7 membered saturated or partially saturated ring containing from 0-2 additional heteroatoms selected from the group consisting of N, O, and S(O)ₒ;

‘p’ represents integers from 0 to 5;

‘n’ represents integers from 0-3;

When A, R₁, R₂, R₃R₄, or R₅ are substituted, the substituents at each occurrence may be independently selected from hydroxyl, oxo, halo, thiol, nitro, amino, cyano, formyl, or substituted or unsubstituted groups selected from amidino, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocyclyl, heterocyclylalkyl, heterocycloxy, heterocyclylalkoxy, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, cycloalkylthio, arylthio, heterocyclylthio, alkylsulfinyl, cycloalkylsulfinyl, arylsulfinyl, heterocyclylsulfinyl, alkylsulfonyl, cycloalkylsulfonyl, arylsulfonyl, heterocyclylsulfonyl, alkylsulfonylamino, cycloalkylsulfonylamino, arylsulfonylamino, heterocyclylsulfonylamino, alkylsulfonyloxy, cycloalkylsulfonyloxy, arylsulfonyloxy, heterocyclylsulfonyloxy, alkoxycarbonylamino, aryloxycarbonylamino, aralkyloxycarbonylamino, aminocarbonylamino, alkylaminocarbonylamino, alkoxyamino, hydroxyl amino, sulfinyl derivatives, sulfonyl derivatives, sulfonic acid and its derivatives.

When the substituents on A, R₁, R₂, R₃R₄ or R₅ are further substituted, the substituents may be selected from one or more groups described above.

In a further preferred embodiment the groups, radicals described above may be selected from:
the “alkyl” group used either alone or in combination with other radicals, denotes a linear or branched radical containing one to six carbons, selected from methyl, ethyl, n-propyl, iso-propyl, n-butyl, sec-butyl, tert-butyl, amyl, t-amyl, n-pentyl, n-hexyl, and the like;
the “alkenyl” group used either alone or in combination with other radicals, is selected from a radical containing from two to six carbons, more preferably groups selected from vinyl, allyl, 2-butenyl, 3-butenyl, 2-pentenyl, 3-pentenyl, 4-pentenyl, 2-hexenyl, 3-hexenyl, 4-hexenyl and the like; the “alkenyl” group includes dienes and trienes of straight and branched chains;
the “cycloalkyl”, or “alicyclic” group used either alone or in combination with other radicals, is selected from a cyclic radical containing three to six carbons, more preferably cyclopropyl, cyclobutyl, cyclopentyl, cyclohexyl and the like;
the “cycloalkenyl” group used either alone or in combination with other radicals, are preferably selected from cyclopropenyl, 1-cyclobutenyl, 2-cylobutenyl, 1-cyclopentenyl, 2-cyclopentenyl, 3-cyclopentenyl, 1-cyclohexenyl, 2-cyclohexenyl, 3-cyclohexenyl and the like; The terms “bicycloalkenyl” means more than one cycloalkenyl groups fused together;
the “alkoxy” group used either alone or in combination with other radicals, is selected from groups containing an alkyl radical, as defined above, attached directly to an oxygen atom, more preferably groups selected from methoxy, ethoxy, n-propoxy, iso-propoxy, n-butoxy, t-butoxy, iso-butoxy, pentyloxy, hexyloxy, and the like;
the “cycloalkoxy” group used either alone or in combination with other radicals, is selected from a cyclic radical containing three to seven carbons, more preferably cyclopropyloxy, cyclobutylxoy, cyclopentyloxy, cyclohexyloxy and the like; The terms “bicycloalkyloxy” means more than one cycloalkyl groups fused together;
the “alkenoxy” group used either alone or in combination with other radicals, is selected from groups containing an alkenyl radical, as defined above, attached to an oxygen atom, more preferably selected from vinyloxy, allyloxy, butenoxy, pentenoxy, hexenoxy, and the like;
the “haloalkyl” group is selected from an alkyl radical, as defined above, suitably substituted with one or more halogens; such as perhaloalkyl, more preferably, perfluoro(C₁-C₆)alkyl such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups;
the “haloalkoxy” group is selected from suitable haloalkyl, as defined above, directly attached to an oxygen atom, more preferably groups selected from fluoromethoxy, chloromethoxy, fluoroethoxy, chloroethoxy and the like;
the spirocyclic group is selected from a cycloalkyl or heterocycloalkyl bicyclic radical, where fusion occurs at single atom.
the bridgehead group is selected from a cycloalkyl or heterocycloalkyl bicyclic radical, where fusion of rings occur across a sequence of atoms.
the “aryl” or “aromatic” group used either alone or in combination with other radicals, is selected from a suitable aromatic system containing one, two or three rings wherein such rings may be attached together in a pendant manner or may be fused, more preferably the groups are selected from phenyl, naphthyl, tetrahydronaphthyl, indane, biphenyl, and the like;
the “aryloxy” group used either alone or in combination with other radicals, is selected from groups containing an aryl radical, as defined above, attached directly to an oxygen atom, more preferably groups selected from phenoxy, naphthyloxy, tetrahydronaphthyloxy, biphenyloxy, and the like;
the “heterocyclyl” or “heterocyclic” group used either alone or in combination with other radicals, is selected from suitable aromatic or non-aromatic radicals containing one or more hetero atoms selected, from O, N or S. The non-aromatic radicals may be saturated, partially saturated or unsaturated mono, bi or tricyclic radicals, containing one or more heteroatoms selected from nitrogen, sulfur and oxygen, more preferably selected from aziridinyl, azetidinyl, pyrrolidinyl, imidazolidinyl, piperidinyl, piperazinyl, 2-oxopiperidinyl, 4-oxopiperidinyl, 2-oxopiperazinyl, 3-oxopiperazinyl, morpholinyl, thiomorpholinyl, 2-oxomorpholinyl, azepinyl, diazepinyl, oxapinyl, thiazepinyl, oxazolidinyl, thiazolidinyl, dihydrothiophene, dihydropyran, dihydrofuran, dihydrothiazole, benzopyranyl, benzopyranonyl, benzodihydrofuranyl, benzodihydrothienyl, pyrazolopyrimidonyl, azaquinazolinoyl, thienopyrimidonyl, quinazolonyl, pyrimidonyl, benzoxazinyl, benzoxazinonyl, benzothiazinyl, benzothiazinonyl, thieno piperidinyl, and the like; the aromatic radicals, may be selected from suitable single or fused mono, bi or tricyclic aromatic heterocyclic radicals containing one or more hetero atoms selected from O, N or; S, more preferably the groups are selected from pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzofuranyl, benzothienyl, indolinyl, indolyl, azaindolyl, azaindolinyl, pyrazolopyrimidinyl, azaquinazolinyl, pyridofuranyl, pyridothienyl, thienopyrimidyl, quinolinyl, pyrimidinyl, pyrazolyl, quinazolinyl, pyridazinyl, triazinyl, benzimidazolyl, benzotriazolyl, phthalazynil, naphthylidinyl, purinyl, carbazolyl, phenothiazinyl, phenoxazinyl, benzoxazolyl, benzothiazolyl and the like;
the groups “heterocycloxy”, “heterocyclylalkoxy” are selected from suitable heterocyclyl, heterocyclylalkyl groups respectively, as defined above, attached to an oxygen atom;
the “acyl” group used either alone or in combination with other radicals, is selected from a radical containing one to eight carbons, more preferably selected from formyl, acetyl, propanoyl, butanoyl, iso-butanoyl, pentanoyl, hexanoyl, heptanoyl, benzoyl and the like, which may be substituted;
the “acyloxy” group used either alone or in combination with other radicals, is selected from a suitable acyl group, as defined above, directly attached to an oxygen atom, more preferably such groups are selected from acetyloxy, propionyloxy, butanoyloxy, iso-butanoyloxy, benzoyloxy and the like;
the “acylamino” group used either alone or in combination with other radicals, is selected from a suitable acyl group as defined earlier, attached to an amino radical, more preferably such groups are selected from CH₃CONH, C₂H₅CONH, C₃H₇CONH, C₄H₉CONH, C₆H₅CONH and the like, which may be substituted;
the “mono-substituted amino” group used either alone or in combination with other radicals, represents an amino group substituted with one group selected from (C₁-C₆)alkyl, substituted alkyl, aryl, substituted aryl or arylalkyl groups as defined earlier, more preferably such groups are selected from methylamine, ethylamine, n-propylamine, n-butylamine, n-pentylamine and the like;
the ‘disubstituted amino” group used either alone or in combination with other radicals, represents an amino group, substituted with two radicals that may be same or different selected from (C₁-C₆)alkyl, substituted alkyl, aryl, substituted aryl, or arylalkyl groups, as defined above, more preferably the groups are selected from dimethylamino, methylethylamino, diethylamino, phenylmethyl amino and the like;
the “arylamino” used either alone or in combination with other radicals, represents an aryl group, as defined above, linked through amino having a free valence bond from the nitrogen atom, more preferably the groups are selected from phenylamino, naphthylamino, N-methyl anilino and the like;
the “oxo” or “carbonyl” group used either alone (—C═O—) or in combination with other radicals such as alkyl described above, for e.g. “alkylcarbonyl”, denotes a carbonyl radical (—C═O—) substituted with an alkyl radical described above such as acyl or alkanoyl;
the “carboxylic acid” group, used alone or in combination with other radicals, denotes a —COOH group, and includes derivatives of carboxylic acid such as esters and amides;
the “ester” group used alone or in combination with other radicals, denotes —COO— group, and includes carboxylic acid derivatives, more preferably the ester moieties are selected from alkoxycarbonyl, such as methoxycarbonyl, ethoxycarbonyl, and the like, which may optionally be substituted; aryloxycarbonyl group such as phenoxycarbonyl, napthyloxycarbonyl, and the like, which may optionally be substituted; aralkoxycarbonyl group such as benzyloxycarbonyl, phenethyloxycarbonyl, napthylmethoxycarbonyl, and the like, which may optionally be substituted; heteroaryloxycarbonyl, heteroaralkoxycarbonyl, wherein the heteroaryl group, is as defined above, which may optionally be substituted; heterocyclyloxycarbonyl, where the heterocyclic group, as defined earlier, which may optionally be substituted;
the “amide” group used alone or in combination with other radicals, represents an aminocarbonyl radical (H₂N—C═O), wherein the amino group is mono- or di-substituted or unsubstituted, more preferably the groups are selected from methyl amide, dimethyl amide, ethyl amide, diethyl amide, and the like;
the “aminocarbonyl” group used either alone or in combination with other radicals, may be selected from ‘aminocarbonyl’, ‘aminocarbonylalkyl”, “n-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl”, and “N-alkyl-N-hydroxyaminocarbonylalkyl”, each of them being optionally substituted. The terms “N-alkylaminocabonyl” and “N,N-dialkylaminocarbonyl” denotes aminocarbonyl radicals, as defined above, which have been substituted with one alkyl radical and with two alkyl radicals, respectively. Preferred are “lower alkylaminocarbonyl” having lower alkyl radicals as described above attached to aminocarbonyl radical. The terms “N-arylaminocarbonyl” and “N-alkyl-N-arylaminocarbonyl” denote amiocarbonyl radicals substituted, respectively, with one aryl radical, or one alkyl, and one aryl radical. The term “aminocarbonylalkyl” includes alkyl radicals substituted with aminocarbonyl radicals;
the “hydroxyalkyl” group used either alone or in combination with other radicals, is selected from an alkyl group, as defined above, substituted with one or more hydroxy radicals, more preferably the groups are selected from hydroxymethyl, hydroxyethyl, hydroxypropyl, hydroxybutyl, hydroxypentyl, hydroxyhexyl and the like;
the “aminoalkyl” group used alone or in combination with other radicals, denotes an amino (—NH₂) moiety attached to an alkyl radical, as defined above, which may be substituted, such as mono- and di-substituted aminoalkyl. The term “alkylamino” used herein, alone or in combination with other radicals, denotes an alkyl radical, as defined above, attached to an amino group, which may be substituted, such as mono- and di-substituted alkylamino;
the “alkoxyalkyl” group used alone or in combination with other radicals, denotes an alkoxy group, as defined above, attached to an alkyl group as defined above, more preferably the groups may be selected from methoxymethyl, ethoxymethyl, methoxyethyl, ethoxyethyl and the like;
the “alkylthio” group used either alone or in combination with other radicals, denotes a straight or branched or cyclic monovalent substituent comprising an alkyl group as defined above, linked through a divalent sulfur atom having a free valence bond from the sulfur atom, more preferably the groups may be selected from methylthio, ethylthio, propylthio,
the “thioalkyl” group used either alone or in combination with other radicals, denotes an alkyl group, as defined above, attached to a group of formula —SR′, where R′ represents hydrogen, alkyl or aryl group, e.g. thiomethyl, methylthiomethyl, phenylthiomethyl and the like, which may be optionally substituted.
the “alkoxycarbonylamino” group used alone or in combination with other radicals, is selected from a suitable alkoxycarbonyl group, as defined above, attached to an amino group, more preferably methoxycarbonylamino, ethoxycarbonylamino, and the like;
the “arylthio” group used either alone or in combination with other radicals, denotes a comprising an aryl group as defined above, linked through a divalent sulfur atom having a free valence bond from the sulfur atom, more preferably the groups may be selected from phenylthio, naphthylthio, tetrahydronaphthylthio, indanethio, biphenylthio, and the like;
the “heterocyclylthio” group used either alone or in combination with other radicals, denotes a comprising an heterocyclyl group as defined above, linked through a divalent sulfur atom having a free valence bond from the sulfur atom, more preferably the groups may be selected from aziridinylthio, azetidinylthio, pyrrolidinylthio, imidazolidinylthio, piperidinylthio, piperazinylthio, 2-oxopiperidinylthio, 4-oxopiperidinylthio, 2-oxopiperazinylthio, 3-oxopiperazinylthio, morpholinylthio, thiomorpholinylthio, 2-oxomorpholinylthio, azepinylthio, diazepinylthio, oxapinylthio, thiazepinylthio, oxazolidinylthio, thiazolidinylthio, dihydrothiophenethio, dihydropyranthio, dihydrofuranthio, dihydrothiazolethio, benzopyranylthio, benzopyranonylthio, benzodihydrofuranylthio, benzodihydrothienylthio, pyrazolopyrimidonylthio, azaquinazolinoylthio, thienopyrimidonylthio, quinazolonylthio, pyrimidonylthio, benzoxazinylthio, benzoxazinonylthio, benzothiazinylthio, benzothiazinonylthio, thieno piperidinylthio, pyridylthio, thienylthio, furylthio, pyrrolylthio, oxazolylthio, thiazolylthio, isothiazolylthio, imidazolylthio, isoxazolylthio, oxadiazolylthio, thiadiazolylthio, triazolylthio, tetrazolylthio, benzofuranylthio, benzothienylthio, indolinylthio, indolylthio, azaindolylthio, azaindolinylthio, pyrazolopyrimidinylthio, azaquinazolinylthio, pyridofuranylthio, pyridothienylthio, thienopyrimidylthio, quinolinylthio, pyrimidinylthio, pyrazolylthio, quinazolinylthio, pyridazinylthio, triazinylthio, benzimidazolylthio, benzotriazolylthio, phthalazynilthio, naphthylidinylthio, purinylthio, carbazolylthio, phenothiazinylthio, phenoxazinylthio, benzoxazolylthio, benzothiazolylthio and the like;
the “alkoxycarbonylamino” group used alone or in combination with other radicals, is selected from a suitable alkoxycarbonyl group, as defined above, attached to an amino group, more preferably methoxycarbonylamino, ethoxycarbonylamino, and the like;
the “aminocarbonylamino”, “alkylaminocarbonylamino”, “dialkylaminocarbonylamino” groups used alone or in combination with other radicals, is a carbonylamino (—CONH₂) group, attached to amino(NH₂), alkylamino group or dialkylamino group respectively, where alkyl group is as defined above;
the “amidino” group used either alone or in combination with other radicals, represents a —C(═NH)—NH₂ radical; the “alkylamidino” group represents an alkyl radical, as described above, attached to an amidino group;
the “alkoxyamino” group used either alone or in combination with other radicals, represents a suitable alkoxy group as defined above, attached to an amino group;
the “hydroxyamino” group used either alone or in combination with other radicals, represents a —NHOH moiety, and may be optionally substituted with suitable groups selected from those described above;
the “sulfenyl” group or “sulfenyl derivatives” used alone or in combination with other radicals, represents a bivalent group, —SO— or RₓSO, where Rₓ is an optionally substituted alkyl, aryl, heteroaryl, heterocyclyl, group selected from those described above;
the “sulfonyl” group or “sulfones derivatives” used either alone or in combination with other radicals, with other terms such as alkylsulfonyl, represents a divalent radical —SO₂—, or RₓSO₂—, where Rₓ is as defined above. More preferably, the groups may be selected from “alkylsulfonyl” wherein suitable alkyl radicals, selected from those defined above, is attached to a sulfonyl radical, such as methylsulfonyl, ethylsulfonyl, propylsulfonyl and the like, “arylsulfonyl” wherein an aryl radical, as defined above, is attached to a sulfonyl radical, such as phenylsulfonyl and the like. The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.
The phrase “therapeutically effective” is intended to qualify the amount of active ingredients used in the treatment of a disease or disorder. This amount will achieve the goal of reducing or eliminating the said disease or disorder.
The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.
As used herein, reference to “treatment” of a patient is intended to include prophylaxis. The term “patient” means all mammals including humans. Examples of patients include humans, cows, dogs, cats, goats, sheep, pigs, and rabbits.

Preferably, the patient is a human.

Suitable groups and substituents on the groups may be selected from those described anywhere in the specification.
Particularly useful compounds may be selected from
2-(2-Methyl-4-(2-phenyl-1-(((4-(trifluoromethyl)benzyl)oxy)imino)ethyl)phenoxy)-1-morpholinoethanone;
1-Morpholino-2-(4-(2-phenyl-1-(((4-(trifluoromethyl)benzyl)oxy)imino)ethyl)phenoxy)ethanone;
2-(2-Methyl-4-(2-phenyl-1-(((4-(trifluoromethyl)benzyl)oxy)imino)ethyl)phenoxy)-1-(4-methylpiperazin-1-yl)ethanone;
1-(4-Methylpiperazin-1-yl)-2-(4-(2-phenyl-1-(((4-(trifluoromethyl)benzyl)oxy)imino)ethyl)phenoxy)ethanone;
2-(2-Methyl-4-(2-phenyl-1-(((4-(trifluoromethyl)benzyl)oxy)imino)ethyl)phenoxy)-N-(2-morpholino-2-oxoethyl)acetamide;
N-(2-Morpholino-2-oxoethyl)-2-(4-(2-phenyl-1-(((4-(trifluoromethyl)benzyl)oxy)imino)ethyl)phenoxy)acetamide;
N-Hydroxy-2-(2-methyl-4-(2-phenyl-1-(((4-(trifluoromethyl)benzyl)oxy)imino)ethyl)phenoxy)acetamide;
tert-Butyl 4-((2-(4-(2-phenyl-1-(((4-(trifluoromethyl)benzyl)oxy)imino)ethyl)phenoxy)acetamido)oxy)piperidine-1-carboxylate;
N-Morpholino-2-(4-(2-phenyl-1-(((4-(trifluoromethyl)benzyl)oxy)imino)ethyl)phenoxy)acetamide;
2-(2-Methyl-4-(2-(thiophen-3-yl)-1-(((4-(trifluoromethyl)benzyl)oxy)imino)ethyl)phenoxy)-1-morpholinoethanone;
1-Morpholino-2-(4-(1-(((4-(trifluoromethyl)benzyl)oxy)imino)ethyl)phenoxy)ethanone;
1-(4-Methylpiperazin-1-yl)-2-(4-(1-(((4-(trifluoromethyl)benzyl)oxy)imino)ethyl)phenoxy)ethanone;
1-Morpholino-2-(4-(1-(((4-(trifluoromethyl)benzyl)oxy)imino)propyl)phenoxy)ethanone
1-(4-Methylpiperazin-1-yl)-2-(4-(1-(((4-(trifluoromethyl)benzyl)oxy)imino)propyl)phenoxy)ethanone;
1-(Piperidin-1-yl)-2-(4-(1-(((4-(trifluoromethyl)benzyl)oxy)imino)propyl)phenoxy)ethanone;
N-Cyclopentyl-2-(4-(1-(((4-(trifluoromethyl)benzyl)oxy)imino)propyl)phenoxy)acetamide;
N-Cyclopropyl-2-(4-(1-(((4-(trifluoromethyl)benzyl)oxy)imino)propyl)phenoxy)acetamide;
4-(2-(4-(1-(((4-(Trifluoromethyl)benzyl)oxy)imino)propyl)phenoxy)acetyl)piperazin-2-one;
N-(2-Hydroxyethyl)-N-phenyl-2-(4-(1-(((4-(trifluoromethyl)benzyl)oxy)imino)propyl)phenoxy)acetamide;
N-Morpholino-2-(4-(1-(((4-(trifluoromethyl)benzyl)oxy)imino)propyl)phenoxy)acetamide;
1-(4-Hydroxypiperidin-1-yl)-2-(4-(1-(((4-(trifluoromethyl)benzyl)oxy)imino)propyl)phenoxy)ethanone;
N-(1,3-Dimethyl-1H-pyrazol-5-yl)-2-(4-(1-(((4-(trifluoromethyl)benzyl)oxy)imino)propyl)phenoxy)acetamide;
2-(2-Methyl-4-(1-(((4-(trifluoromethyl)benzyl)oxy)imino)butyl)phenoxy)-1-morpholinoethanone;
2-(2-Methyl-4-(1-(((4-(trifluoromethyl)benzyl)oxy)imino)butyl)phenoxy)-1-(4-methylpiperazin-1-yl)ethanone;
2-(4-(1-((Benzyloxy)imino)-2-phenylethyl)phenoxy)-1-morpholinoethanone;
2-(2-Methyl-4-(1-(((4-methylbenzyl)oxy)imino)-2-phenylethyl)phenoxy)-1-morpholinoethanone;
2-(4-(1-(((4-Methylbenzyl)oxy)imino)-2-phenylethyl)phenoxy)-1-morpholinoethanone;
2-(4-(1-(((4-Fluorobenzyl)oxy)imino)-2-phenylethyl)-2-methylphenoxy)-1-morpholinoethanone;
2-(4-(1-(((4-Fluorobenzyl)oxy)imino)-2-phenylethyl)phenoxy)-1-morpholinoethanone;
2-(4-(1-(((4-Fluorobenzyl)oxy)imino)-2-phenylethyl)phenoxy)-1-(4-methylpiperazin-1-yl)ethanone;
2-(4-(1-(((4-Fluorobenzyl)oxy)imino)-2-phenylethyl)phenoxy)-N-(2-morpholino-2-oxoethyl)acetamide;
2-(4-(1-(((4-Chlorobenzyl)oxy)imino)-2-phenylethyl)-2-methylphenoxy)-1-morpholinoethanone;
2-(4-(1-(((4-Chlorobenzyl)oxy)imino)-2-phenylethyl)phenoxy)-1-morpholinoethanone;
2-(2-Methyl-4-(2-phenyl-1-(((4-(trifluoromethoxy)benzyl)oxy)imino)ethyl)phenoxy)-1-morpholinoethanone;
1-Morpholino-2-(4-(2-phenyl-1-(((4-(trifluoromethoxy)benzyl)oxy)imino)ethyl)phenoxy)ethanone;
2-(4-(1-(((4-Methoxybenzyl)oxy)imino)-2-phenylethyl)-2-methylphenoxy)-1-morpholinoethanone;
2-(4-(1-(((4-Methoxybenzyl)oxy)imino)-2-phenylethyl)phenoxy)-1-morpholinoethanone;
2-(4-(1-(((4-Methoxybenzyl)oxy)imino)-2-phenylethyl)-2-methylphenoxy)-1-(4-methylpiperazin-1-yl)ethanone;
2-(4-(1-(((4-Methoxybenzyl)oxy)imino)-2-phenylethyl)phenoxy)-1-(4-methylpiperazin-1-yl)ethanone;
2-(4-(1-(((4-Methoxybenzyl)oxy)imino)-2-phenylethyl)-2-methylphenoxy)-1-(piperidin-1-yl)ethanone;
2-(4-(1-(((4-Methoxybenzyl)oxy)imino)-2-phenylethyl)phenoxy)-1-(piperidin-1-yl)ethanone;
2-(4-(1-(((4-(Methylsulfonyl)benzyl)oxy)imino)propyl)phenoxy)-1-morpholinoethanone;
1-(4-Methylpiperazin-1-yl)-2-(4-(1-(((4-(methylsulfonyl)benzyl)oxy)imino)propyl)phenoxy)ethanone;
1-Morpholino-2-(4-(2-phenyl-1-((pyridin-2-ylmethoxy)imino)ethyl)phenoxy)ethanone;
2-(4-(1-((2-(1H-Indol-1-yl)ethoxy)imino)propyl)phenoxy)-1-morpholinoethanone;
2-(4-(1-((2-(1H-Indol-1-yl)ethoxy)imino)propyl)phenoxy)-1-(4-methylpiperazin-1-yl)ethanone;
3-Methyl-2-((((1-(4-(2-morpholino-2-oxoethoxy)phenyl)propyl idene)amino)oxy)methyl)quinazolin-4(3H)-one;
2-(4-(1-(((5-Ethylpyrimidin-2-yl)oxy)imino)propyl)phenoxy)-1-morpholinoethanone;
2-(4-(1-(((5-Ethylpyrimidin-2-yl)oxy)imino)propyl)phenoxy)-1-(4-methylpiperazin-1-yl)ethanone;
2-(4-(1-(((5-Methyl-2-phenyloxazol-4-yl)methoxy)imino)propyl)phenoxy)-1-(4-methylpiperazin-1-yl)ethanone;
2-(4-(1-(((5-Methyl-2-phenyloxazol-4-yl)methoxy)imino)propyl)phenoxy)-1-morpholinoethanone;
2-(4-(1-(((3-(tert-Butyl)-1-(p-tolyl)-1H-pyrazol-5-yl)methoxy)imino)propyl)phenoxy)-1-morpholinoethanone;
2-(4-(1-(((3-(tert-Butyl)-1-(p-tolyl)-1H-pyrazol-5-yl)methoxy)imino)propyl)phenoxy)-1-(4-methylpiperazin-1-yl)ethanone;
1-(Piperidin-1-yl)-2-((5-(((4-(trifluoromethyl)benzyl)oxy)imino)-5,6,7,8-tetrahydronaphthalen-2-yl)oxy)ethanone;
1-Morpholino-2-((5-(((4-(trifluoromethyl)benzyl)oxy)imino)-5,6,7,8-tetrahydronaphthalen-2-yl)oxy)ethanone;
1-(4-Methylpiperazin-1-yl)-2-((5-(((4-(trifluoromethyl)benzyl)oxy)imino)-5,6,7,8-tetrahydronaphthalen-2-yl)oxy)ethanone;
N-Morpholino-2-((5-(((4-(trifluoromethyl)benzyl)oxy)imino)-5,6,7,8-tetrahydronaphthalen-2-yl)oxy)acetamide;
N-(Piperidin-1-yl)-2-((5-(((4-(trifluoromethyl)benzyl)oxy)imino)-5,6,7,8-tetrahydronaphthalen-2-yl)oxy)acetamide;
N-(5-Chlorobenzo[d]oxazol-2-yl)-2-((5-(((4-(trifluoromethyl)benzyl)oxy)imino)-5,6,7,8-tetrahydronaphthalen-2-yl)oxy)acetamide;
N-(4-Methylpyrimidin-2-yl)-2-((5-(((4-(trifluoromethyl)benzyl)oxy)imino)-5,6,7,8-tetrahydronaphthalen-2-yl)oxy)acetamide;
N-(1,3-Dimethyl-1H-pyrazol-5-yl)-2-((5-(((4-(trifluoromethyl)benzyl)oxy)imino)-5,6,7,8-tetrahydronaphthalen-2-yl)oxy)acetamide;
tert-Butyl 4-((2-((5-(((4-(trifluoromethyl)benzyl)oxy)imino)-5,6,7,8-tetrahydronaphthalen-2-yl)oxy)acetamido)oxy)piperidine-1-carboxylate;
N-(2-Isocyanophenyl)-2-((5-(((4-(trifluoromethyl)benzyl)oxy)imino)-5,6,7,8-tetrahydronaphthalen-2-yl)oxy)acetamide;
2-((4-((Naphthalen-2-ylmethoxy)imino)chroman-7-yl)oxy)-1-(piperidin-1-yl)ethanone;
2-((4-((Benzyloxy)imino)chroman-7-yl)oxy)-1-(piperidin-1-yl)ethanone;
2-((4-(((4-Fluorobenzyl)oxy)imino)chroman-7-yl)oxy)-1-(piperidin-1-yl)ethanone;
1-(Piperidin-1-yl)-2-((4-(((4-(trifluoromethyl)benzyl)oxy)imino)chroman-7-yl)oxy)ethanone;
2-((4-(((4-Methoxybenzyl)oxy)imino)chroman-7-yl)oxy)-1-(piperidin-1-yl)ethanone;
2-((4-(((4-Methoxybenzyl)oxy)imino)chroman-7-yl)oxy)-1-morpholinoethanone;
2-((4-(((4-Methoxybenzyl)oxy)imino)chroman-7-yl)oxy)-1-(4-methylpiperazin-1-yl)ethanone;
8-(2-((5-(((4-(Trifluoromethyl)benzyl)oxy)imino)-5,6,7,8-tetrahydronaphthalen-2-yl)oxy)acetyl)-1-oxa-3,8-diazaspiro[4.5]decan-2-one;
8-(2-((4-(((4-(Trifluoromethyl)benzyl)oxy)imino)chroman-7-yl)oxy)acetyl)-1-oxa-3,8-diazaspiro[4.5]decan-2-one;
8-(2-((4-(((4-Methoxybenzyl)oxy)imino)chroman-7-yl)oxy)acetyl)-1-oxa-3,8-diazaspiro[4.5]decan-2-one;
8-(2-((4-((Naphthalen-2-ylmethoxy)imino)chroman-7-yl)oxy)acetyl)-1-oxa-3,8-diazaspiro[4.5]decan-2-one;
8-(2-((4-(((6-(Trifluoromethyl)pyridin-3-yl)methoxy)imino)chroman-7-yl)oxy)acetyl)-1-oxa-3,8-diazaspiro[4.5]decan-2-one;
8-(2-((4-(((1-Methyl-1H-indol-6-yl)methoxy)imino)chroman-7-yl)oxy)acetyl)-1-oxa-3,8-diazaspiro[4.5]decan-2-one;
8-(2-(4-(1-(((4-Methoxybenzyl)oxy)imino)propyl)phenoxy)acetyl)-1-oxa-3,8-diazaspiro[4.5]decan-2-one;
8-(2-(4-(1-(((4-(Trifluoromethyl)benzyl)oxy)imino)propyl)phenoxy)acetyl)-1-oxa-3,8-diazaspiro[4.5]decan-2-one;
8-(2-(4-(1-(((4-(Trifluoromethyl)benzyl)oxy)imino)ethyl)phenoxy)acetyl)-1-oxa-3,8-diazaspiro[4.5]decan-2-one;
8-(2-(4-(1-(((4-Methoxybenzyl)oxy)imino)ethyl)phenoxy)acetyl)-1-oxa-3,8-diazaspiro[4.5]decan-2-one;
8-(2-(4-(1-((4-Methoxyphenoxy)imino)ethyl)phenoxy)acetyl)-1-oxa-3,8-diazaspiro[4.5]decan-2-one;
8-(2-(4-(1-((4-(Trifluoromethyl)phenoxy)imino)ethyl)phenoxy)acetyl)-1-oxa-3,8-diazaspiro[4.5]decan-2-one;
1-(6-Azaspiro[2.5]octan-6-yl)-2-(4-(1(((4(trifluoromethyl)benzyl)oxy)imino)ethyl)phenoxy)ethanone;
1-(6-Azaspiro[2.5]octan-6-yl)-2-(4-(1-(((4-(trifluoromethyl)benzyl)oxy)imino)propyl)phenoxy)ethanone;
2-(4-(2-Phenyl-1-(((4-(trifluoromethyl)benzyl)oxy)imino)ethyl)phenoxy)-1-(6-azaspiro[2.5]octan-6-yl)ethanone;
2-(4-(1-(((4-Methoxybenzyl)oxy)imino)propyl)phenoxy)-1-(6-azaspiro[2.5]octan-6-yl)ethanone;
2-(4-(1-(((4-Fluorobenzyl)oxy)imino)propyl)phenoxy)-1-(6-azaspiro[2.5]octan-6-yl)ethanone;
2-(4-(1-((3-(6-Azaspiro[2.5]octan-6-yl)propoxy)imino)propyl)phenoxy)-1-(piperidin-1-yl)ethanone;
2-(4-(1-((2-(6-Azaspiro[2.5]octan-6-yl)ethoxy)imino)propyl)phenoxy)-1-(piperidin-1-yl)ethanone;
2-(4-(1-((2-(Methyl(phenyl)amino)ethoxy)imino)propyl)phenoxy)-1-(piperidin-1-yl)ethanone;
2-(4-(1-((2-((4-Fluorophenyl)(methyl)amino)ethoxy)imino)propyl)phenoxy)-1-(piperidin-1-yl)ethanone;
2-(4-(1-((2-((4-Fluorophenyl)(methyl)amino)ethoxy)imino)propyl)phenoxy)-1-morpholinoethanone;
2-(4-(1-((2-((4-Fluorophenyl)(methyl)amino)ethoxy)imino)propyl)phenoxy)-1-thiomorpholinoethanone;
2-(4-(1-((Naphthalen-2-ylmethoxy)imino)propyl)phenoxy)-1-(6-azaspiro[2.5]octan-6-yl)ethanone;
2-(4-(1-((Naphthalen-2-ylmethoxy)imino)pentyl)phenoxy)-1-(6-azaspiro[2.5]octan-6-yl)ethanone;
2-(4-(1-(((3,4-Dimethylbenzyl)oxy)imino)pentyl)phenoxy)-1-(6-azaspiro[2.5]octan-6-yl)ethanone;
2-(4-(1-(((3,4-Dimethylbenzyl)oxy)imino)-3-methoxypropyl)phenoxy)-1-(6-azaspiro[2.5]octan-6-yl)ethanone;
2-(4-(1-((Dibenzo[b,d]thiophen-3-ylmethoxy)imino)-3-methoxypropyl)phenoxy)-1-(6-azaspiro[2.5]octan-6-yl)ethanone;
2-(4-(1-((Dibenzo[b,d]thiophen-3-ylmethoxy)imino)-3-methoxypropyl)phenoxy)-1-(piperidin-1-yl)ethanone;
N-((1-Methylpiperidin-4-yl)oxy)-2-(4-(2-morpholino-1-(((4-(trifluoromethyl)benzyl)oxy)imino)ethyl)phenoxy)ethanethioamide;
2-(4-(1-(((3,5-Bis(trifluoromethyl)benzyl)oxy)imino)-2-morpholinoethyl)phenoxy)-N-((1-methylpiperidin-4-yl)oxy)ethanethioamide;
2-(4-(1-(((3,5-Bis(trifluoromethyl)benzyl)oxy)imino)-2-morpholinoethyl)phenoxy)-N-((1-methylpiperidin-4-yl)oxy)acetamide;
2-(4-(1-(((3,5-Bis(trifluoromethyl)benzyl)oxy)imino)-2-(piperidin-1-yl)ethyl)phenoxy)-2-methyl-N-((1-methylpiperidin-4-yl)methyl)propanamide;
2-methyl-N-((1-Methylpiperidin-4-yl)methyl)-2-(4-(1-(((tetrahydro-2H-pyran-4-yl)methoxy)imino)ethyl)phenoxy)propanamide;
N-((1-Methylpiperidin-4-yl)methyl)-2-(4-(1-(((tetrahydro-2H-pyran-4-yl)methoxy)imino)ethyl)phenoxy)propanamide;
2-(4-(2-Methoxy-1-(((4-(trifluoromethyl)benzyl)oxy)imino)ethyl)phenoxy)-N-((1-methylpiperidin-4-yl)methyl)ethanethioamide;
2-(4-(2-Methoxy-1-(((4-(trifluoromethyl)benzyl)oxy)imino)ethyl)phenoxy)-1-(piperidin-1-yl)ethanethione;
2-(4-(1-(([1,1′-Biphenyl]-4-ylmethoxy)imino)-2-methoxyethyl)phenoxy)-1-(piperidin-1-yl)ethanethione;
2-(4-(2-Methoxy-1-(((4′-(trifluoromethyl)-[1,1′-biphenyl]-4-yl)methoxy)imino)ethyl)phenoxy)-1-(piperidin-1-yl)ethanethione;
2-(4-(2-Methoxy-1-(((4′-(trifluoromethyl)-[1,1′-biphenyl]-4-yl)methoxy)imino)ethyl)phenoxy)-1-(4-methylpiperazin-1-yl)ethanethione;
N-(Cyclohexylmethyl)-3-(4-(1-(((4-(trifluoromethyl)benzyl)oxy)imino)propyl)phenyl)propanethioamide;
N-(Cyclohexylmethyl)-3-(4-(2-phenyl-1-(((4-(trifluoromethyl)benzyl)oxy)imino)ethyl)phenyl) propanethioamide;
N-((1-Methylpiperidin-4-yl)methyl)-2-(4-(2-phenyl-1-(((4-(trifluoromethyl)benzyl)oxy)imino)ethyl)phenoxy)ethanethioamide;
2-(4-(2-Cyclohexyl-1-(((4-(trifluoromethyl)benzyl)oxy)imino)ethyl)phenoxy)-N-((1-methylpiperidin-4-yl)methyl)ethanethioamide;
N-((1-Methylpiperidin-4-yl)methyl)-2-(4-(2-morpholino-1-(((4-(trifluoromethyl)benzyl)oxy)imino)ethyl)phenoxy)ethanethioamide;
1-(4-Allylpiperazin-1-yl)-2-(4-(1-((pyridin-4-ylmethoxy)imino)propyl)phenoxy)ethanone;
1-(4-Allylpiperazin-1-yl)-2,2-difluoro-2-(4-(1-((pyridin-4-ylmethoxy)imino)propyl)phenoxy)ethanone;
N-(Cyclohexylmethyl)-2,2-difluoro-N-methyl-2-(4-(1-((pyridin-4-ylmethoxy)imino)propyl)phenoxy)acetamide;
N-(Cyclohexylmethyl)-2,2-difluoro-2-(4-(1-((pyridin-4-ylmethoxy)imino)propyl)phenoxy)acetamide;
2-(4-(1-((Benzyloxy)imino)propyl)phenoxy)-N-(cyclohexylmethyl)-2,2-difluoroacetamide;
N-(Cyclohexylmethyl)-2,2-difluoro-2-(4-(1-(((4-(trifluoromethyl)benzyl)oxy)imino)propyl)phenoxy)acetamide;
N-(Cyclohexylmethyl)-2-(4-(1-(((4-(trifluoromethyl)benzyl)oxy)imino)propyl)phenoxy)ethanethioamide;
2-(4-(1-(((1,2,3,4-Tetrahydronaphthalen-1-yl)oxy)imino)propyl)phenoxy)-1-(4-(3-(trifluoromethyl)phenyl)piperazin-1-yl)ethanone;
2-(4-(1-((Cyclohexyloxy)imino)propyl)phenoxy)-1-(4-(3-(trifluoromethyl)phenyl)piperazin-1-yl)ethanone;
2-(4-(1-((Benzyloxy)imino)propyl)phenoxy)-1-(4-(3-(trifluoromethyl)phenyl)piperazin-1-yl)ethanone;
2-(4-(1-(((2-Fluorobenzyl)oxy)imino)propyl)phenoxy)-1-(4-(3-(trifluoromethyl)phenyl)piperazin-1-yl)ethanone;
2-(4-(1-((Pyridin-4-ylmethoxy)imino)propyl)phenoxy)-1-(4-(3-(trifluoromethyl)phenyl)piperazin-1-yl)ethanone;
1-(4-Phenylpiperazin-1-yl)-2-(4-(1-((pyridin-4-ylmethoxy)imino)propyl)phenoxy)ethanone;
8-(2-(4-(1-(((1,2,3,4-Tetrahydronaphthalen-1-yl)oxy)imino)propyl)phenoxy)acetyl)-1-oxa-3,8-diazaspiro[4.5]decan-2-one;
1-(Piperidin-1-yl)-2-(4-(1-(((1,2,3,4-tetrahydronaphthalen-1-yl)oxy)imino)propyl)phenoxy)ethanone;
1-(4-(2-Hydroxyethyl)piperazin-1-yl)-2-(4-(1-(((1,2,3,4-tetrahydronaphthalen-1-yl)oxy)imino)propyl)phenoxy)ethanone;
2-Hydroxy-1-(4-(2-(4-(1-(((1,2,3,4-tetrahydronaphthalen-1-yl)oxy)imino)propyl)phenoxy)acetyl)piperazin-1-yl)ethanone;
2-Hydroxy-1-(4-(2-(4-(1-(((2-methylbenzyl)oxy)imino)propyl)phenoxy)acetyl)piperazin-1-yl)ethanone;
2-Hydroxy-1-(4-(2-(4-(1-(((2-(trifluoromethyl)benzyl)oxy)imino) propyl)phenoxy)acetyl)piperazin-1-yl)ethanone;
2-(4-(1-(((5-Fluoro-2-(trifluoromethyl)benzyl)oxy)imino)propyl)phenoxy)-1-(4-(2-hydroxyacetyl)piperazin-1-yl)ethanone;
N-Methyl-2-((1-methyl-5-((phenoxyimino)methyl)-1H-indol-2-yl)oxy)-N-(2-oxo-2-(piperidin-1-yl)ethyl)acetamide;
2-((5-(((Benzyloxy)imino)methyl)-1-methyl-1H-indol-2-yl)oxy)-N-methyl-N-(2-oxo-2-(piperidin-1-yl)ethyl)acetamide;
N-Methyl-2-((1-methyl-5-((((4-(trifluoromethyl)benzyl)oxy)imino)methyl)-1H-indol-2-yl)oxy)-N-(2-oxo-2-(piperidin-1-yl)ethyl)acetamide;
2-((1-Methyl-5-((((4-(trifluoromethyl)benzyl)oxy)imino)methyl)-1H-indol-2-yl)oxy)-N-(2-oxo-2-(piperidin-1-yl)ethyl)acetamide;
2-((5-((((4-Fluorobenzyl)oxy)imino)methyl)-1-methyl-1H-indol-2-yl)oxy)-N-(2-oxo-2-(piperidin-1-yl)ethyl)acetamide;
N-Methyl-2-((1-methyl-4-(1-(((3-phenylallyl)oxy)imino)propyl)-1H-pyrrol-2-yl)oxy)-N-(2-oxo-2-(piperidin-1-yl)ethyl)acetamide;
N-Benzyl-2-((1-methyl-4-(1-(((3-phenylallyl)oxy)imino)propyl)-1H-pyrrol-2-yl)oxy)acetamide;
2-((1-Methyl-4-(1-(((3-phenylallyl)oxy)imino)propyl)-1H-pyrrol-2-yl)oxy)-N-(4-(trifluoromethyl)benzyl)acetamide;
2-((1-Methyl-4-(1-((3-phenylpropoxy)imino)propyl)-1H-pyrrol-2-yl)oxy)-N-(4-(trifluoromethyl)benzyl)acetamide;
2-((1-Methyl-4-(1-((3-phenylpropoxy)imino)propyl)-1H-pyrrol-2-yl)oxy)-N-(naphthalen-2-ylmethyl)acetamide;
2-(4-(1-((Dibenzo[b,d]thiophen-3-ylmethoxy)imino)-3-methoxypropyl)phenoxy)-N-methyl-N-(2-oxo-2-(piperidin-1-yl)ethyl)acetamide;
2-(4-(1-((Benzo[b]thiophen-6-ylmethoxy)imino)-3-methoxypropyl)phenoxy)-N-methyl-N-(2-oxo-2-(piperidin-1-yl)ethyl)acetamide;
2-(4-(1-((Benzyloxy)imino)-3-methoxypropyl)phenoxy)-N-methyl-N-(2-oxo-2-(piperidin-1-yl)ethyl)acetamide;
2-((5-(1-((Benzyloxy)imino)-3-methoxypropyl)pyridin-2-yl)oxy)-N-methyl-N-(2-oxo-2-(piperidin-1-yl)ethyl)acetamide;
2-((4-(1-((Benzyloxy)imino)propyl)-1-methyl-1H-pyrrol-2-yl)oxy)-N-methyl-N-(2-oxo-2-(piperidin-1-yl)ethyl)acetamide;
2-(4-(1-((2-(6-Azaspiro[2.5]octan-6-yl)ethoxy)imino)ethyl)phenoxy)-N-((3,4-dihydro-2H-pyran-4-yl)methyl)acetamide;
2-(4-(1-((Benzyloxy)imino)ethyl)phenoxy)-N-((3,4-dihydro-2H-pyran-4-yl)methyl)acetamide;
N-((3,4-Dihydro-2H-pyran-4-yl)methyl)-2-(4-(1-(((4-(trifluoromethyl)benzyl)oxy)imino)ethyl)phenoxy)acetamide;
N-((3,4-Dihydro-2H-pyran-4-yl)methyl)-2-(4-(1-(((6-(trifluoromethyl)pyridin-3-yl)methoxy)imino)ethyl)phenoxy)acetamide;
N-((3,4-Dihydro-2H-pyran-4-yl)methyl)-2-(4-(2-hydroxy-1-(((6-(trifluoromethyl)pyridin-3-yl)methoxy)imino)ethyl)phenoxy)acetamide;
N-((3,4-dihydro-2H-pyran-4-yl)methyl)-2-(4-(2-methoxy-1-(((6-(trifluoromethyl)pyridin-3-yl)methoxy)imino)ethyl)phenoxy)acetamide;
N-((3,4-Dihydro-2H-pyran-4-yl)methyl)-2-(4-(1-(((6-(trifluoromethyl)pyridin-3-yl)methoxy)imino)butyl)phenoxy)acetamide;
N-((3,6-Dihydro-2H-pyran-4-yl)methyl)-2-(4-(1-(((4-(trifluoromethyl)benzyl)oxy)imino)ethyl)phenoxy)acetamide;
N-((3,6-Dihydro-2H-pyran-4-yl)methyl)-2-(4-(1-((naphthalen-2-ylmethoxy)imino)ethyl)phenoxy)acetamide;
N-((3,6-Dihydro-2H-pyran-4-yl)methyl)-2-((5-(1-((naphthalen-2-ylmethoxy)imino)ethyl)pyridin-2-yl)oxy)acetamide;
N-((3,6-Dihydro-2H-pyran-4-yl)methyl)-2-((1-methyl-4-(1-((naphthalen-2-ylmethoxy)imino)ethyl)-1H-pyrrol-2-yl)oxy)acetamide;
N-((3,6-Dihydro-2H-pyran-4-yl)methyl)-2-((1-methyl-4-(1-((quinolin-7-ylmethoxy)imino)ethyl)-1H-pyrrol-2-yl)oxy)acetamide;
2-((4-(1-((2-(6-Azaspiro[2.5]octan-6-yl)ethoxy)imino)ethyl)-1-methyl-1H-pyrrol-2-yl)oxy)-N-((3,6-dihydro-2H-pyran-4-yl)methyl)acetamide;
2-(4-(1-((2-(6-Azaspiro[2.5]octan-6-yl)ethoxy)imino)ethyl)phenoxy)-N-((3,6-dihydro-2H-pyran-4-yl)methyl)acetamide;
N-((3,6-Dihydro-2H-pyran-4-yl)methyl)-2-((4-(((4-(trifluoromethyl)benzyl)oxy)imino)chroman-7-yl)oxy)acetamide;
N-((3,6-Dihydro-2H-pyran-4-yl)methyl)-2-(4-(1-(((4-(trifluoromethyl)benzyl)oxy)imino)propyl)phenoxy)acetamide;
N-((3,6-Dihydro-2H-pyran-4-yl)methyl)-2-(4-(1-(((4-fluorobenzyl)oxy)imino)propyl)phenoxy)acetamide;
N-((3,6-Dihydro-2H-pyran-4-yl)methyl)-2-(4-(1-(((4-fluorobenzyl)oxy)imino)propyl)phenoxy)ethanethioamide;
N-((3,6-Dihydro-2H-pyran-4-yl)methyl)-2-(4-(1-(((4-methoxybenzyl)oxy)imino)propyl)phenoxy)ethanethioamide;
N-((3,6-Dihydro-2H-pyran-4-yl)methyl)-2-(4-(1-(((4-methoxybenzyl)oxy)imino)-2-phenylethyl)phenoxy)ethanethioamide;
N-((3,6-Dihydro-2H-pyran-4-yl)methyl)-2-(4-(1-(((4-methoxybenzyl)oxy)imino)-2-phenylethyl)phenoxy)acetamide;
2-(4-(1-((Bicyclo[2.2.2]octan-2-yloxy)imino)ethyl)phenoxy)-1-(piperidin-1-yl)ethanone;
2-(4-(1-((Bicyclo[2.2.2]octan-2-yloxy)imino)-2-phenylethyl)phenoxy)-1-(piperidin-1-yl)ethanone;
2-(4-(1-((Bicyclo[2.2.2]octan-2-yloxy)imino)-2-cyclohexylethyl)phenoxy)-1-(piperidin-1-yl)ethanone;
2-(4-(1-((Bicyclo[2.2.2]octan-2-yloxy)imino)-2-morpholinoethyl)phenoxy)-1-(piperidin-1-yl)ethanone;
2-((5-(1-((Bicyclo[2.2.2]octan-2-yloxy)imino)-2-morpholinoethyl)pyridin-2-yl)oxy)-1-(piperidin-1-yl)ethanone;
2-((8-((Bicyclo[2.2.2]octan-2-yloxy)imino)-5,6,7,8-tetrahydroisoquinolin-3-yl)oxy)-1-(piperidin-1-yl)ethanone;
2-(4-(1-((3-(Bicyclo[2.2.2]octan-2-yloxy)propoxy)imino)propyl)phenoxy)-N-(cyclohexylmethyl)acetamide;
2-(4-(1-((3-(Bicyclo[2.2.2]octan-2-yloxy)propoxy)imino)propyl)phenoxy)-1-morpholinoethanone;
2-(4-(1-((3-(Bicyclo[2.2.2]octan-2-yloxy)propoxy)imino)propyl)phenoxy)-N-phenylacetamide;
2-(4-(1-((3-(Bicyclo[2.2.2]octan-2-yloxy)propoxy)imino)propyl)phenoxy)-N-(2-cyanophenyl)acetamide;
2-(4-(1-((Bicyclo[2.2.2]octan-2-yloxy)imino)propyl)phenoxy)-N-(2-cyanophenyl)acetamide;
2-(4-(1-((Bicyclo[2.2.2]octan-2-yloxy)imino)propyl)phenoxy)-N-((1-methylpiperidin-4-yl)methyl)acetamide;
2-(4-(1-((Bicyclo[2.2.2]octan-2-yloxy)imino)ethyl)phenoxy)-N-((1-methylpiperidin-4-yl)methyl)acetamide.

The novel compounds of this invention may be prepared using the reactions and techniques as shown in schemes below and described in this section. The reactions are performed in solvents appropriate to the reagents and materials employed and are suitable for the transformations being affected. It is understood by those skilled in the art that the nature and order of the synthetic steps presented may be varied for the purpose of optimizing the formation of the compounds of the present invention. It will also be well appreciated that one or more of the reactants may be protected and deprotected for facile synthesis by techniques known to persons skilled in the art. It will also be appreciated that one or more of the compounds of the present invention may exist in stereoisomeric and/or diastereomeric forms. Such stereoisomers and/or diastereoisomers as well as their optical antipodes are to be construed to be within the scope of the present invention. It will also be well appreciated that one or more of these compounds may be converted to their salts and other derivatives based on the specific groups present on the compounds, which can be well comprehended by persons skilled in the art. Such salts and/or other derivatives, as the case may be should also be construed to be within the scope of the present invention.

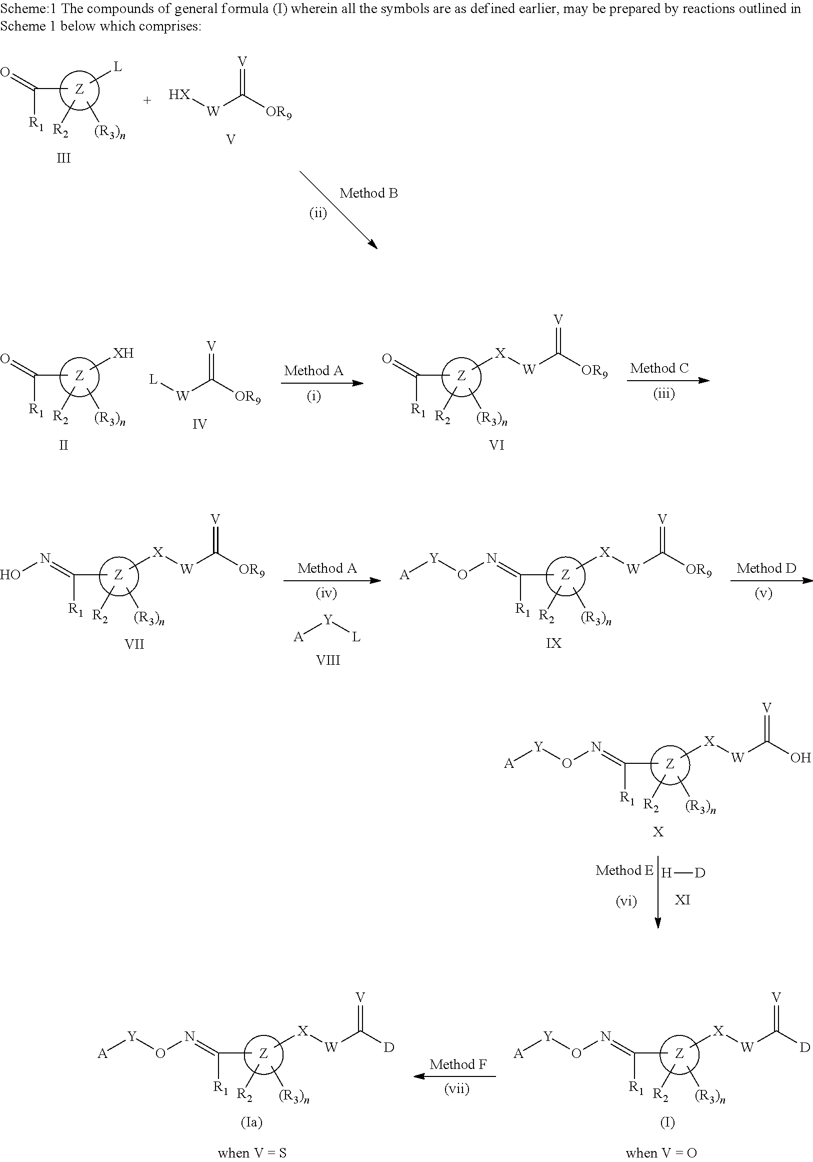

i. Compounds of general formula VI wherein R₉ represents C₁-C₆ linear or branched alkyl or aralkyl, and all other symbols are as defined earlier may be prepared by coupling of compounds of general formula II wherein all the symbols are as defined earlier and compounds of general formula IV whereas ‘L’ represents a suitable leaving group and all other symbols are as defined earlier using suitable base and suitable reaction medium by means of the methods available in the literature for standard nucleophilic substitution reaction;
ii. Alternatively compounds of general formula VI wherein all the symbols are as defined earlier may be prepared by coupling of compounds of formula III whereas all the symbols are as defined earlier and compounds of general formula V wherein all other symbols are as defined using suitable base and suitable reaction medium by means of the methods available in the literature for standard nucleophilic substitution reaction;
iii. Compounds of general formula VII wherein all the symbols are as defined earlier may be prepared by reacting compounds of general formula VI wherein all the symbols are as defined earlier with hydroxylamine hydrochloride in presence of suitable base and suitable solvents;
iv. Compounds of formula IX wherein all the symbols are as defined earlier may be prepared by coupling of compounds of formula VII wherein all the symbols are as defined earlier and compounds of general formula VIII wherein ‘L’ represents a suitable leaving group and all other symbols are as defined earlier using suitable base and suitable reaction medium;
v. Compounds of general formula X wherein all the symbols are as defined earlier may be prepared by the hydrolysis of compounds of general formula IX wherein all the symbols are as defined earlier under suitable condition;
vi. Compounds of formula (I) wherein all the symbols are as defined earlier may be prepared by coupling of compounds of formula X wherein all symbols are as defined earlier and compounds of formula XI wherein all symbols are as defined using suitable methods available in the literature for standard peptide coupling;
vii. Compounds of formula (Ia) wherein V represents S and all the symbols are as defined earlier may be prepared by thionation of compounds of formula (Ia) wherein all symbols are as defined earlier using suitable methods available in the literature for sulfur insertion;

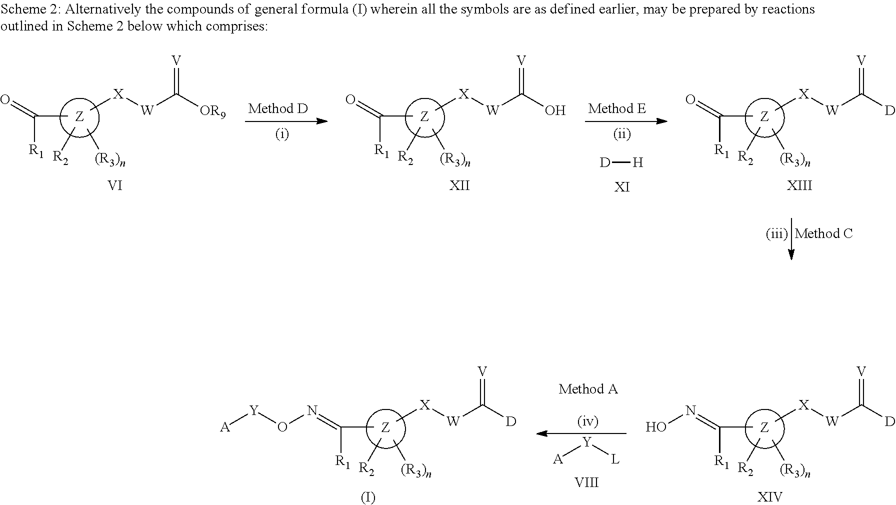

i. Compounds of general formula XII wherein all the symbols are as defined earlier may be prepared by the hydrolysis of compounds of general formula VI wherein all the symbols are as defined earlier under suitable condition;
ii. Compounds of formula XIII wherein all the symbols are as defined earlier may be prepared by coupling of compounds of formula XII wherein all symbols are as defined earlier and compounds of formula XI wherein all symbols are as defined earlier using suitable methods available in the literature for standard peptide coupling;
iii. Compounds of general formula XIV wherein all the symbols are as defined earlier may be prepared by reacting compounds of general formula XIII wherein all the symbols are as defined earlier with hydroxylamine hydrochloride in presence of suitable base and suitable solvents;
iv. Compounds of formula (I) wherein all the symbols are as defined earlier may be prepared by coupling of compounds of formula XIV wherein all the symbols are as defined earlier and compounds of general formula VIII wherein all the symbols are as defined earlier using suitable base and suitable reaction medium.
   Method A: The compounds of formula VI, IX and (I) wherein all the symbols are as defined earlier may be prepared by appropriate starting materials as described in Scheme 1 and Scheme 2 using suitable inorganic base(s) such as NaOH, KOH, K₂CO₃, Cs₂CO₃ and the like or organic base(s) such as pyridine, triethyl amine, diisopropyl ethylamine and the like. The reaction may be carried out neat or in presence of suitable protic solvent(s) such as methanol, ethanol, butanol and the like or suitable aprotic solvent(s) such as dimethyl formamide, tetrahydrofuran, dichloromethane and the like or suitable mixtures thereof. The reaction may be carried out at a temperature in the range 0° C. to reflux temperature of the solvent(s) used and the reaction time may range from 1 to 48 hours.
   Method B: The compounds of formula VI wherein all the symbols are as defined earlier may be prepared by using appropriate starting materials as described in Scheme 1 using suitable inorganic base(s) such as NaOH, KOH, K₂CO₃, Cs₂CO₃ and the like or suitable organic base(s) such as pyridine, triethyl amine, diisopropyl ethylamine and the like. Alternatively the compounds of formula VI wherein all the symbols are as defined earlier may also be prepared by using suitable palladium based catalyst such as palladium acetate, Pd(Ph₃P)₄ and the like and with or without organic ligand such as BINAP and the like. The reaction may be carried out neat or in presence of suitable protic solvent(s) such as methanol, ethanol, butanol and the like or suitable aprotic solvent(s) such as dimethyl formamide, toluene, tetrahydrofuran, dichloromethane and the like or mixtures thereof. The reaction may be carried out at a temperature in the range 0° C. to reflux temperature of the solvent(s) used and the reaction time may range from 1 to 48 hours.
   Method C: The compounds of the formula VII and XIV wherein all the symbols are as defined earlier may be prepared by reacting appropriate ketones as described in Scheme 1 and Scheme 2 with hydroxylamine hydrochloride in the presence of a base(s) like NaOH, NaOAc, pyridine and the like. The reaction may be carried out in presence of suitable solvent(s) such as methanol, ethanol, butanol, water and the like or suitable mixtures thereof. The reaction may be carried out at a temperature in the range 0° C. to reflux temperature of the solvent(s) used and the reaction time may range from 1 to 48 hours.
   Method D: The compounds of the formula X and XII wherein all the symbols are as defined earlier may be prepared by hydrolyzing appropriate esters as described in Scheme 1 and Scheme 2 using suitable base(s) e.g., NaOH, LiOH, KOH and the like. Reaction may be conducted in suitable solvent(s) such as methanol, ethanol, THF, water and the like or the mixtures thereof. The reaction may be carried out at a temperature in the range 20° C. to reflux temperature of the solvent(s) used and the reaction time may range from 1 to 48 hours.
   Method E: The compounds of the formula (I) and XIII wherein all the symbols are as defined earlier may be prepared by coupling reaction of appropriate acid and appropriate amine as described in scheme 1 and scheme 2 under suitable conditions such as those described in Tetrahedron, 2005, 61(46), 10827-10852 with suitable modifications and alterations as are well known to a skilled person. The reaction may be carried out in presence of reagents(s) such as N-(3-dimethylaminopropyl)-N′-ethylcarbodimide hydrochloride (EDCl) & 1-Hydroxybenzotriazole (HOBT), and the like. The reaction may be carried in suitable solvent(s) such as dimethyl formamide, tetrahydrofuran, dichloromethane and the like or mixtures thereof. The reaction may be carried out at a temperature in the range 0° C. to reflux temperature of the solvent(s) used and the reaction time may range from 1 to 48 hours.
   Method F: The compounds of the formula Ia wherein ‘V’ represents ‘S’ and all other symbols are as defined earlier may be prepared by reacting compounds of the formula I with reagents with elemental sulfur donating capacity or sulfur itself. The reactions may be carried out using suitable solvents and conditions as reported in literature. Preferred method for sulfur insertion is to treat compounds of formula I with Phosphorous pentasulfide or Lawesson's reagent (2,4-bis(4-methoxyphenyl)-1,3,2,4-dithiadiphosphetane-2,4-disulfide) in a solvents such as THF, toluene or pyridine etc. The reaction may be carried out at a temperature in the range 0° C. to reflux temperature of the solvent(s) used and the reaction time may range from 1 to 48 hours.
   The pharmaceutical composition is provided by employing conventional techniques. Preferably the composition is in unit dosage form containing an effective amount of the active component, that is, the compounds of formula (I) according to this invention.
   The quantity of active component, that is, the compounds of formula (I) according to this invention, in the pharmaceutical composition and unit dosage form thereof may be varied or adjusted widely depending upon the particular application method, the potency of the particular compound and the desired concentration. Generally, the quantity of active component will range between 0.5% to 90% by weight of the composition.
   The compounds of the present invention can be used either alone or in combination with one or more therapeutic agents selected from insulin, insulin derivatives and mimetics, insulin secretagogues, insulin sensitizers, biguanide agents, alpha-glucosidase inhibitors, insulinotropic sulfonylurea receptor ligands, meglitinides, GLP-1 (glucagon like peptide-1), GLP-1 analogs, DPPIV (dipeptidyl peptidase IV) inhibitors, GPR-119 activators, sodium-dependent glucose co-transporter (SGLT2) inhibitors, PPAR modulators, non-glitazone type PPAR.delta agonist, HMG-CoA reductase inhibitors, cholesterol-lowering drugs, rennin inhibitors, anti-thrombotic and anti-platelet agents and anti-obesity agents or pharmaceutically acceptable salts thereof. The invention is explained in greater detail by the examples given below, which are provided by way of illustration only and therefore should not be construed to limit the scope of the invention.

1H NMR spectral data given in the examples (vide infra) are recorded using a 400 MHz spectrometer (Bruker A VANCE-400) and reported in S scale. Until and otherwise mentioned the solvent used for NMR is CDCl₃ using tetramethyl silane as the internal standard.

### Example 1

### N-((Tetrahydro-2H-pyran-4-yl)methyl)-2-(4-(1-(((4-(trifluoromethyl)benzyl)oxy)imino)propyl)phenoxy)acetamide

### Step 1: Ethyl 2-(2-methyl-4-(2-phenylacetyl)phenoxy)acetate

To a solution of 1-(4-hydroxy-3-methylphenyl)-2-phenylethanone (25 g, 0.11 moles) in DMF (125 mL), potassium carbonate (30.5 gm, 0.22 moles) and ethyl bromo acetate (20.3 gm, 0.121 moles) were added and the reaction mixture was stirred at 50° C. for 3 hours. The reaction mixture was poured into ice cold water and extracted with ethyl acetate. The combined ethyl acetate extract was washed with water & brine, dried over sodium sulphate and evaporated under reduced pressure to yield 31 gm (90%) of product as thick liquid.

¹H NMR: 1.27 (t, J=7.1 Hz, 3H), 2.31 (s, 3H), 4.23-4.30 (m, 4H), 4.64 (s, 2H), 6.67 (d, J=8.2 Hz, 1H), 7.24-7.34 (m, 5H), 7.82-7.85 (m, 2H).

### Step 2: Ethyl 2-(4-(1-(hydroxyimino)-2-phenylethyl)-2-methylphenoxy)acetate

To a solution of the product of step 1 (11 g, 0.035 moles) in methanol (100 ml), hydroxyl amine hydrochloride (4.89 g, 0.035 moles) and a solution of sodium acetate (5.78 g, 0.035 moles) in water (50 ml) were added and the reaction mixture was refluxed for 1 hour. The solvents were evaporated under reduced pressure. The residue was dissolved in water and extracted with ethyl acetate. The combined ethyl acetate extract was washed with water & brine, dried over sodium sulphate and evaporated under reduced pressure to yield 10.4 gm (90%) of product as solid.

¹H NMR: 1.29 (t, J=7.1 Hz; 3H), 2.26 (s, 3H), 4.17 (s, 2H), 4.24 (q, J=7.1 Hz, 2H), 4.62 (s, 2H), 6.61 (d, J=8.8 Hz, 2H), 7.16-7.20 (m, 2H), 7.35 (d, J=8.5 Hz, 2H), 7.47 (s, 2H).

### Step 3: Ethyl 2-(2-methyl-4-(2-phenyl-1-(((4-(trifluoromethyl)benzyl)oxy)imino)ethyl)phenoxy)acetate

To a solution of the product of step 2 (0.50 g, 2.09 mmoles) in DMF (5 mL), cesium carbonate (1.02 gm, 3.14 mmoles) and 4-(trifluoromethyl)benzyl bromide (0.5 gm, 2.09 mmoles) were added and the reaction mixture was stirred at 25° C. for 3 hours. The reaction mixture was poured into ice cold water and extracted with ethyl acetate. The combined ethyl acetate extract was washed with water & brine, dried over sodium sulphate and evaporated under reduced pressure to yield 0.55 gm (58%) of product as thick liquid.

¹H NMR: 1.27 (t, J=7.1 Hz, 3H), 2.26 (s, 3H), 4.13 (s, 2H), 4.23 (q, J=7.1 Hz, 2H), 4.61 (s, 2H), 5.27 (s, 2H), 6.60 (d, J=8.5 Hz, 1H), 7.15-7.19 (m, 5H), 7.21-7.25 (m, 3H), 7.49 (s, 1H), 7.54 (d, J=7.9 Hz, 2H).

### Step 4: 2-(2-Methyl-4-(2-phenyl-1-(((4-(trifluoromethyl)benzyl)oxy)imino)ethyl)phenoxy)acetic acid

To a solution of the product of step 3 (0.5 g, 1.03 mmoles) in THF (10 ml), a solution of lithium hydroxide (86 mg, 2.06 mmoles) in water (5 ml) was added and the reaction mixture was stirred at 25° C. for 3 hours. The solvents were evaporated under reduced pressure. The residue was dissolved in water, acidified with 1N HCl and extracted with ethyl acetate. The combined ethyl acetate extract was washed with water & brine, dried over sodium sulphate and evaporated under reduced pressure to yield 0.45 gm (95%) of product as solid.

¹H NMR: 2.15 (s, 3H), 4.16 (s, 2H), 4.68 (s, 2H), 5.31 (s, 2H), 6.76 (d, J=8.8 Hz, 1H), 7.15-7.25 (m, 5H), 7.41 (dd, J=8.4 & 2.4 Hz, 1H), 7.50 (d, J=1.6 Hz, 1H), 7.55 (d, J=8.0 Hz, 2H), 7.70 (d, J=8.4 Hz, 2H).

### Step 5: 2-(2-Methyl-4-(2-phenyl-1-(((4-(trifluoromethyl)benzyl)oxy)imino)ethyl)phenoxy)-1-morpholinoethanone

To a solution of the product of step 4 (0.5 g, 1.09 mmoles) in DMF (5 mL), morpholine (99 mg, 1.1-5 mmoles), HOBT (50 mg), EDC.HCl (225 mg, 1.30 mmoles) and DMAP (50 mg) were added and reaction mixture was stirred at 25° C. for 16 hours. The reaction mixture was poured into ice cold water and extracted with ethyl acetate. The combined ethyl acetate extract was washed with water & brine, dried over sodium sulphate and evaporated under reduced pressure. The crude product was purified by column chromatography using ethyl acetate:Hexane (1:1) as eluent to yield 0.35 g (57%) of product as white solid.

¹H NMR (DMSO-d₆): 2.14 (s, 3H), 3.42-3.43 (m, 4H), 3.54-3.57 (m, 4H), 4.15 (s, 2H), 4.84 (s, 2H), 5.31 (s, 2H), 6.79 (d, J=8.4 Hz, 1H), 7.14-7.16 (m, 3H), 7.20-7.24 (m, 2H), 7.40 (d, J=8.8 Hz, 1H), 7.49 (s, 1H), 7.55 (d, J=8.0 Hz, 2H), 7.70 (d, J=8.0 Hz, 2H).

### Example 2

### N-Hydroxy-2-(2-methyl-4-(2-phenyl-1-(((4-(trifluoromethyl)benzyl)oxy)imino)ethyl)phenoxy)acetamide

To a solution of ethyl 2-(2-methyl-4-(2-phenyl-1-(((4-(trifluoromethyl)benzyl)oxy)imino)ethyl)phenoxy)acetate (500 mg, 1.03 mmoles) in THF (7.5 ml), a solution of NH₂OH (68 mg, 2.06 mmoles) in MeOH (8 ml) was added followed by the addition of water (0.8 ml) and NaCN (15 mg, 0.3 mmoles) at 25° C. The reaction mixture was stirred at 25° C. for 24 hours. The reaction mixture was poured into ice cold water and extracted by ethyl acetate (20 ml x 3). The combined ethyl acetate extract was washed with water and brine, dried over sodium sulphate and evaporated under reduced pressure. The crude product was purified by column chromatography (Eluent: 15% ethyl acetate in hexane, 230-400 mesh silica gel) to yield 150 mg of title product as white solid.

### Example 3

### 2-(2-Methyl-4-(2-phenyl-1-(((4-(trifluoromethyl)benzyl)oxy)imino)ethyl)phenoxy)-N-(2-morpholino-2-oxoethyl)acetamide

### Step 1: Ethyl 2-(2-(2-methyl-4-(2-phenyl-1-(((4-(trifluoromethyl)benzyl)oxy)imino)ethyl)phenoxy)acetamido)acetate

To a solution of product of step 4 of example 1 (300 mg, 0.66 mmoles) in DMF (2 mL), glycine ethyl ester hydrochloride (96 mg, 0.69 mmoles), HOBT (138 mg, 1.02 mmoles), EDCI (151 mg, 0.79 mmoles) and N-ethyl morpholine (250 μl, 1.97 mmoles) were added and reaction mixture was stirred at 25° C. for 18 hours under nitrogen atmosphere. The reaction mixture was poured into ice cold water and extracted by ethyl acetate (20 ml x 3). The combined ethyl acetate extract was washed with water and brine, dried over sodium sulphate and evaporated under reduced pressure to yield 320 mg product as thick liquid.

¹H NMR (DMSO-d₆): 1.13 (t, J=7.2 Hz, 3H), 2.20 (s, 3H), 3.85 (d, J=6.0 Hz, 2H), 4.03 (q, J=7.0 Hz, 2H), 4.17 (s, 2H), 4.56 (s, 2H), 5.31 (s, 2H), 6.80 (d, J=8.8 Hz, 1H), 7.13-7.16 (m, 3H), 7.20-7.24 (m, 2H), 7.42 (dd, J=8.8 & 2.0 Hz, 1H), 7.51 (d, J=1.6 Hz, 1H), 7.55 (d, J=8.0 Hz, 2H), 7.70 (d, J=8.0 Hz, 2H), 8.26 (t, J=5.8 Hz, NH)

Yield: 90%

### Step 2: 2-(2-(2-Methyl-4-(2-phenyl-1-(((4-(trifluoromethyl)benzyl)oxy)imino)ethyl)phenoxy)acetamido)acetic acid

To a solution of the product of step 1 (360 mg, 0.66 mmoles) in ethanol (6 ml), a solution of sodium hydroxide (53 mg, 1.32 mmoles) in water (2 ml) was added and the reaction mixture was stirred at ambient temperature for 4 hours. The solvents were evaporated under reduced pressure. The residue was dissolved in water and acidified with 1N HCl. White solid separated which was filtered and washed with water & dried over P₂O₅ under vacuum to give 570 mg of title product

¹H NMR (DMSO-d₆): 2.20 (s, 3H), 3.77 (d, J=6.0 Hz, 2H), 4.16 (s, 2H), 4.53 (s, 2H), 5.31 (s, 2H), 6.82 (d, J=8.8 Hz, 1H), 7.12-7.16 (m, 3H), 7.20-7.24 (m, 2H), 7.41 (dd, J=8.6 & 2.2 Hz, 1H), 7.50 (d, J=1.6 Hz, 1H); 7.54 (d, J=8.0 Hz, 2H), 7.70 (d, J=8.0 Hz, 2H), 8.12 (t, J=6.0 Hz, NH), 12.8 (bs, OH).

Yield: 82%

### Step 3: 2-(2-Methyl-4-(2-phenyl-1-(((4-(trifluoromethyl)benzyl)oxy)imino)ethyl)phenoxy)-N-(2-morpholino-2-oxoethyl)acetamide

To a solution of the product of step 2 (280 mg, 0.55 mmoles) in DMF (2 mL), morpholine (50 μL, 0.57 mmoles), HOBT (115 mg, 0.85 mmoles), EDCI (125 mg, 0.65 mmoles) and N-ethyl morpholine (207 μL, 1.64 mmoles) were added and reaction mixture was stirred at 25° C. for 3 hours under nitrogen atmosphere. The reaction mixture was poured into ice cold water and extracted by ethyl acetate (20 ml x 3). The combined ethyl acetate extract was washed with water and brine, dried over sodium sulphate and evaporated under reduced pressure. The crude product was purified by recrystallisation in 50 Ethyl acetate: Hexane (1:1) (20 ml) to yield 180 mg product as white solid.

¹H NMR: 2.21 (s, 3H), 3.39-3.44 (m, 4H), 3.52-3.55 (m, 4H), 4.00 (d, J=5.2 Hz, 2H), 4.17 (s, 2H), 4.56 (s, 2H), 5.31 (s, 2H), 6.85 (d, J=8.8 Hz, 1H), 7.13-7.16 (m, 3H), 7.20-7.24 (m, 2H), 7.42 (dd, J=8.8 & 2.0 Hz, 1H), 7.51 (s, 1H), 7.55 (d, J=8.0 Hz, 2H), 7.70 (d, J=8.0 Hz, 2H), 7.93 (t, J=5.0 Hz, NH).

Yield: 57%

The following examples were prepared following the general procedures given in the Example 1-3 with suitable modifications, alterations and other process variations which are within the scope of a person skilled in the art.

### Example 4

### 1-Morpholino-2-(4-(2-phenyl-1-(((4-(trifluoromethyl)benzyl)oxy)imino)ethyl)phenoxy)ethanone

¹H NMR (DMSO-d₆): 3.41 (bs, 4H), 3.54-3.57 (m, 4H), 4.16 (s, 2H), 4.82 (s, 2H), 5.31 (s, 2H), 6.87 (d, J=8.8 Hz, 2H), 7.13-7.16 (m, 3H), 7.20-7.24 (m, 2H), 7.52-7.59 (m, 4H), 7.70 (d, J=8.4 Hz, 2H).

Yield: 84%

### Example 5

### 2-(2-Methyl-4-(2-phenyl-1-(((4-(trifluoromethyl)benzyl)oxy)imino)ethyl)phenoxy)-1-(4-methylpiperazin-1-yl)ethanone

¹H NMR: 2.15 (s, 6H), 2.22 (m, 2H), 2.29 (m, 2H), 3.39-3.42 (m, 4H), 4.15 (s, 2H), 4.81 (m, 2H), 5.30 (m, 2H), 6.78 (d, J=8.8 Hz, 1H), 7.15 (d, J=7.2 Hz, 3H), 7.21 (d, J=7.2 Hz, 2H), 7.39-7.42 (dd, J=8.8 & 8.4 Hz, 1H), 7.49 (d, J=2.0 Hz, 1H), 7.55 (d, J=8.4 Hz, 2H), 7.71 (d, J=8.4 Hz, 2H).

Yield: 76%

### Example 6

### 1-(4-Methylpiperazin-1-yl)-2-(4-(2-phenyl-1-(((4-(trifluoromethyl)benzyl)oxy)imino)ethyl)phenoxy)ethanone

¹H NMR: 2.27 (s, 3H), 2.35-2.38 (m, 4H), 3.55 (t, J=5.0 Hz, 2H), 3.62 (t, J=4.6 Hz, 2H), 4.14 (s, 2H), 4.66 (s, 2H), 5.27 (s, 2H), 6.88 (d, J=8.4 Hz, 2H), 7.15-7.25 (m, 5H), 7.40 (d, J=8.0 Hz, 2H), 7.56-7.59 (m, 4H).

Yield: 58%

### Example 7

### N-(2-Morpholino-2-oxoethyl)-2-(4-(2-phenyl-1-(((4-(trifluoromethyl)benzyl)oxy)imino)ethyl)phenoxy)acetamide

¹H NMR (DMSO-d₆): 3.40-3.41 (m, 4H), 3.52-3.54 (m, 4H), 4.00 (d, J=5.2 Hz, 2H), 4.17 (s, 2H), 4.54 (s, 2H), 5.31 (s, 2H), 6.93 (d, J=8.8 Hz, 2H), 7.14-7.16 (m, 3H), 7.20-7.24 (m, 2H), 7.55-7.62 (m, 4H), 7.70 (d, J=8.0 Hz, 2H), 8.08 (t, J=5.0 Hz, NH).

Yield: 77%

### Example 8

### tert-Butyl 4-((2-(4-(2-phenyl-1-(((4-(trifluoromethyl)benzyl)oxy)imino)ethyl)phenoxy)acetamido)oxy)piperidine-1-carboxylate

¹H NMR (DMSO-d₆); 1.37 (bs, 11H), 1.70 (s, 2H), 3.05 (d, J=2.4 Hz, 2H), 3.55 (d, J=4.0 Hz, 2H), 3.91 (m, 1H), 4.17 (s, 2H), 4.48 (s, 2H), 5.31 (s, 2H), 6.91 (d, J=8.8 Hz, 2H), 7.14 (t, J=7.2 Hz, 3H), 7.22 (t, J=7.4 Hz, 2H), 7.61-7.55 (m, 4H), 7.72 (d, J=6.8 Hz, 2H), 11.24 (s, 1H).

Yield: 10%

### Example 9

### N-Morpholino-2-(4-(2-phenyl-1-(((4-(trifluoromethyl)benzyl)oxy)imino)ethyl)phenoxy)acetamide

¹H NMR (DMSO-d₆): 2.75 (t, J=4.8 Hz, 4H), 3.60 (t, J=4.6 Hz, 4H), 4.17 (d, J=4.8 Hz, 2H), 4.43 (s, 1H), 4.86 (s, 1H), 5.31 (s, 2H), 6.82 (d, J=8.8 Hz, 1H), 6.90 (d, J=9.2 Hz, 1H), 7.13-7.17 (m, 3H), 7.21-7.25 (m, 2H), 7.56-7.62 (m, 4H), 7.71 (d, J=8.0 Hz, 2H), 8.82 (s, 0.5H), 9.19 (s, 0.5H).

Yield: 43%

### Example 10

### 2-(2-Methyl-4-(2-(thiophen-3-yl)-1-(((4-(trifluoromethyl)benzyl)oxy)imino)ethyl)phenoxy)-1-morpholinoethanone

¹H NMR (DMSO-d₆): 2.15 (s, 3H), 3.43 (bs, 4H), 3.54-3.57 (m, 4H), 4.11 (s, 2H), 4.85 (s, 2H), 5.30 (s, 2H), 6.80 (d, J=8.8 Hz, 1H), 6.89 (d, J=5.2 Hz, 1H), 7.12 (m, 1H), 7.38-7.40 (m, 1H), 7.42 (dd, J=8.6 & 1.8 Hz, 1H), 7.50 (s, 1H), 7.55 (d, J=8.0 Hz, 2H), 7.70 (d, J=8.0 Hz, 2H).

Yield: 79%

### Example 11

### 1-Morpholino-2-(4-(1-(((4-(trifluoromethyl)benzyl)oxy)imino)ethyl)phenoxy)ethanone

¹H NMR (DMSO-d₆): 2.21 (s, 3H), 3.43 (s, 4H), 3.59 (d, J=8.0 Hz, 4H), 4.85 (s, 2H), 5.26 (s, 2H), 6.93-6.90 (m, 2H), 7.56-7.53 (m, 2H), 7.60 (d, J=8.0 Hz, 2H), 7.73 (d, J=8.0 Hz, 2H).

Yield: 51%

### Example 12

### 1-(4-Methylpiperazin-1-yl)-2-(4-(1-(((4-(trifluoromethyl)benzyl)oxy)imino)ethyl)phenoxy)ethanone

¹H NMR (DMSO-d₆); 2.04 (s, 3H,) 2.21 (s, 3H), 2.31 (s, 4H), 3.42 (bs, 4H) 4.83 (s, 2H), 5.25 (s, 2H), 6.91 (d, J=8.4 Hz, 2H), 7.56 (d, J=8.8 Hz, 2H), 7.60 (d, J=7.6 Hz, 2H), 7.73 (d, J=7.6 Hz, 2H),

Yield: 69%

### Example 13

### 1-Morpholino-2-(4-(1-(((4-(trifluoromethyl)benzyl)oxy)imino)propyl)phenoxy)ethanone

¹H NMR (DMSO-d₆): 1.04 (t, 3H), 2.76-2.65 (m, 2H), 3.43 (s, 4H), 3.59-3.55 (m, 4H), 4.85 (s, 2H), 5.25 (s, 2H), 6.93 (d, J=9.2 Hz, 2H), 7.55 (d, J=8.8 Hz, 2H), 7.60 (d, J=8.0 Hz, 2H), 7.74 (d, J=8.0 Hz, 2H).

Yield: 72%

### Example 14

### 1-(4-Methylpiperazin-1-yl)-2-(4-(1-(((4-(trifluoromethyl)benzyl)oxy)imino)propyl)phenoxy)ethanone

¹H NMR (DMSO-d₆): 1.04 (t, J=7.6 Hz, 3H), 1.97 (s, 3H), 2.24 (bs, 2H), 2.48 (bs, 2H), 2.76-2.70 (m, 2H), 3.42 (t, J=4.8 Hz, 4H), 4.83 (s, 2H), 5.24 (s, 2H), 6.91 (d, 2H), 7.55 (d, J=9.2 Hz, 2H) 7.60 (d, J=8.0 Hz, 2H), 7.74 (d, J=8.0 Hz, 2H),

Yield: 34%

### Example 15

### 1-(Piperidin-1-yl)-2-(4-(1-(((4-(trifluoromethyl)benzyl)oxy)imino)propyl)phenoxy)ethanone

¹H NMR (DMSO-d₆): 1.04 (t, J=7.2 Hz, 3H), 1.41 (bs, 2H), 1.56 (d, J=20.8 Hz, 4H), 2.74-2.66 (m, 2H), 3.38 (s, 4H), 4.81 (s, 2H), 5.24 (s, 2H), 6.91 (d, J=8.4 Hz, 2H), 7.67-7.53 (m, 4H), 7.74 (d, J=8.0 Hz, 2H).

Yield: 52%

### Example 16

### N-Cyclopentyl-2-(4-(1-(((4-(trifluoromethyl)benzyl)oxy)imino)propyl)phenoxy)acetamide

¹H NMR (DMSO-d₆): 1.04 (t, J=7.2 Hz, 3H), 1.38-1.50 (m, 4H), 1.59-1.63 (m, 2H), 1.75-1.81 (m, 2H), 2.70 (q, J=7.6 Hz, 2H), 4.02 (q, J=7.0 Hz 1H), 4.45 (s, 2H), 5.25 (s, 2H), 6.93 (d, J=8.8 Hz, 2H), 7.55 (dd, J=8.8 & 2.0 Hz, 2H), 7.58 (d, J=8.4 Hz, 2H), 7.72 (d, J=8.4 Hz, 2H), 7.96 (d, J=7.6 Hz, 1H).

Yield: 40%

### Example 17

### N-Cyclopropyl-2-(4-(1-(((4-(trifluoromethyl)benzyl)oxy)imino)propyl)phenoxy)acetamide

¹H NMR (DMSO-d₆): 0.44-0.48 (m, 2H), 0.59-0.64 (m, 2H), 1.04 (t, J=7.6 Hz 3H), 2.68-2.49 (m, 1H), 2.70 (q, J=7.6 Hz, 2H), 4.44 (s, 2H), 5.25 (s, 2H), 6.92 (d, J=8.8 Hz, 2H), 7.55-7.59 (m, 4H), 7.71 (d, J=8.0 Hz, 2H), 8.12 (d, J=3.6 Hz 1H).

Yield: 50%

### Example 18

### 4-(2-(4-(1-(((4-(Trifluoromethyl)benzyl)oxy)imino)propyl)phenoxy)acetyl)piperazin-2-one

¹H NMR (DMSO-d₆): 1.04 (t, J=7.4 Hz, 3H), 2.70 (q, J=7.4 Hz, 2H), 3.16 (brs, 2H), 3.58-3.65 (m, 2H), 3.92 (s, 1H), 4.06 (s, 1H), 4.87 (d, J=8.4 Hz, 2H), 5.24 (s, 2H), 6.91 (d, J=8.8 Hz, 2H), 7.55 (dd, J=8.0 & 2.8 Hz, 2H), 7.58 (d, J=8.0 Hz, 2H), 7.71 (d, J=8.0 Hz, 2H), 8.09 (bd, 1H).

Yield: 33%

### Example 19

### N-(2-Hydroxyethyl)-N-phenyl-2-(4-(1-(((4-(trifluoromethyl)benzyl)oxy)imino)propyl)phenoxy)acetamide

¹H NMR (DMSO-d₆): 1.04 (t, J=0.7.6 Hz, 3H), 2.70 (q, J=7.6 Hz, 2H), 3.29 (t, J=5.8 Hz, 2H), 4.25 (t, J=5.6 Hz, 2H), 4.80 (s, 2H), 5.25 (s, 2H), 5.64 (t, J=6.0 Hz, 1H), 6.50-6.54 (m, 1H), 6.57 (d, J=7.6 Hz, 2H), 6.90 (d, J=8.8 Hz, 2H), 7.04-7.07 (m, 2H), 7.52 (d, J=8.8 Hz, 2H), 7.58 (d, J=8.0 Hz, 2H), 7.71 (d, J=8.4 Hz, 2H).

Yield: 27%

### Example 20

### N-(2-Hydroxyethyl)-N-phenyl-2-(4-(1-(((4-(trifluoromethyl)benzyl)oxy)imino)propyl)phenoxy)acetamide

¹H NMR (DMSO-d₆): 1.04 (t, J=7.6 Hz, 3H), 2.70 (q, J=7.6 Hz, 2H), 3.29 (t, J=5.8 Hz, 2H), 4.25 (t, J=5.6 Hz, 2H), 4.80 (s, 2H), 5.25 (s, 2H), 5.64 (t, J=6.0 Hz, 1H), 6.50-6.54 (m, 1H), 6.57 (d, J=7.6 Hz, 2H), 6.90 (d, J=8.8 Hz, 2H), 7.04-7.07 (m, 2H), 7.52 (d, J=8.8 Hz, 2H), 7.58 (d, J=8.0 Hz, 2H), 7.71 (d, J=8.4 Hz, 2H).

Yield: 27%

### Example 21

### 1-(4-Hydroxypiperidin-1-yl)-2-(4-(1-(((4-(trifluoromethyl)benzyl)oxy)imino)propyl)phenoxy)ethanone

¹H NMR (DMSO-d₆): 1.04 (t, J=7.4 Hz, 3H), 1.20-1.24 (m, 1H), 1.35 (bd, 1H), 1.66-1.76 (m, 2H), 2.70 (q, J=7.4 Hz, 2H), 3.01 (t, J=10.0 Hz, 1H), 3.15 (t, J=10.4 Hz, 1H), 3.66-3.71 (m, 2H), 3.83-3.86 (m, 1H), 4.74 (d, J=4.0 Hz, 1H), 4.82 (d, J=2.4 Hz, 2H), 5.24 (s, 2H), 6.89 (d, J=8.8 Hz, 2H), 7.53 (d, J=8.8 Hz, 2H), 7.58 (d, J=8.0 Hz, 2H), 7.71 (d, J=8.0 Hz, 2H).

Yield: 29%

### Example 22

### N-(1,3-Dimethyl-1H-pyrazol-5-yl)-2-(4-(1-(((4-(trifluoromethyl)benzyl)oxy)imino)propyl)phenoxy)acetamide

¹H NMR (DMSO-d₆): 1.05 (t, J=7.6 Hz, 3H), 2.08 (s, 3H), 2.72 (q, J=7.4 Hz, 2H), 3.54 (s, 3H), 4.77 (s, 2H), 5.26 (s, 2H), 5.96 (s, 1H), 6.99 (d, J=8.8 Hz, 2H), 7.59 (d, J=8.8 Hz, 4H), 7.73 (d, J=8.0 Hz, 2H), 10.08 (s, 1H).

Yield: 25%

### Example 23

### 2-(2-Methyl-4-(1-(((4-(trifluoromethyl)benzyl)oxy)imino)butyl)phenoxy)-1-morpholinoethanone

¹H NMR (DMSO-d₆): 0.87 (t, J=7.2 Hz, 3H), 1.43-1.49 (m, 2H), 2.17 (s, 3H), 2.71 (t, J=7.6 Hz, 2H), 3.43-3.45 (m, 4H), 3.55-3.59 (m, 4H), 4.86 (s, 2H), 5.24 (s, 2H), 6.83 (d, J=8.8 Hz, 1H), 7.35-7.37 (dd, J=8.4 & 8.8 Hz, 1H), 7.42 (d, J=1.6 Hz, 1H), 7.58 (d, J=8.0 Hz, 2H), 7.72 (d, J=8.0 Hz, 2H).

Yield: 86%

### Example 24

### 2-(2-Methyl-4-(1-(((4-(trifluoromethyl)benzyl)oxy)imino)butyl)phenoxy)-1-(4-methylpiperazin-1-yl)ethanone

¹H NMR (DMSO-d₆): 0.87 (t, J=7.6 Hz, 3H), 1.41-1.51 (m, 2H), 2.17 (s, 6H), 2.24 (s, 2H), 2.31 (s, 2H), 2.71 (t, J=7.6 Hz, 2H), 3.37-3.43 (m, 4H), 4.84 (s, 2H), 5.24 (s, 2H), 6.82 (d, J=8.4 Hz, 1H), 7.35-7.37 (dd, J=8.4 & 8.4 Hz, 1H), 7.42 (d, J=1.2 Hz, 1H), 7.58 (d, J=8.0 Hz, 2H), 7.72 (d, J=8.0 Hz, 2H).

Yield: 83%

### Example 25

### 2-(4-(1-((Benzyloxy)imino)-2-phenylethyl)phenoxy)-1-morpholinoethanone

¹H NMR: 3.56-3.63 (m, 8H), 4.13 (s, 2H), 4.66 (s, 2H), 5.25 (s, 2H), 6.852 (d, J=2.8 Mz, 2H), 7.14-7.24 (m, 5H), 7.27-7.36 (m, 5H), 7.56-7.59 (m, 2H).

Yield: 95%

### Example 26

### 2-(2-Methyl-4-(1-(((4-methylbenzyl)oxy)imino)-2-phenylethyl)phenoxy)-1-morpholinoethanone

¹H NMR (DMSO-d₆): 2.14 (s, 3H), 2.28 (s, 3H), 3.42 (bs, 4H), 3.53-3.57 (m, 4H), 4.10 (s, 2H), 4.83 (s, 2H), 5.15 (s, 2H), 6.78 (d, J=8.8 Hz, 1H), 7.12-7.22 (comp, 7H), 7.24 (d, J=8.0 Hz, 2H), 7.38 (dd, J=8.6 & 1.8 Hz, 1H), 7.49 (d, J=1.2 Hz, 1H).

Yield: 85%

### Example 27

### 2-(4-(1-(((4-Methylbenzyl)oxy)imino)-2-phenylethyl)phenoxy)-1-morpholinoethanone

¹H NMR (DMSO-d₆): 2.28 (s, 3H), 3.41 (bs, 4H), 3.52-3.57 (m, 4H), 4.11 (s, 2H), 4.81 (s, 2H), 5.15 (s, 2H), 6.85 (d, J=9.2 Hz, 2H), 7.11-7.16 (m, 5H), 7.18-7.22 (m, 2H), 7.25 (d, J=8.0 Hz, 2H), 7.56 (d, J=8.8 Hz, 2H).

Yield: 84%

### Example 28

### 2-(4-(1-(((4-Fluorobenzyl)oxy)imino)-2-phenylethyl)-2-methylphenoxy)-1-morpholinoethanone

¹H NMR (DMSO-d₆): 2.15 (s, 3H), 3.42 (bs, 4H), 3.52-3.57 (m, 4H), 4.11 (s, 2H), 4.84 (s, 2H), 5.18 (s, 2H), 6.78 (d, J=8.8 Hz, 1H), 7.12-7.22 (m, 7H), 7.39-7.43 (m, 3H), 7.49 (S, 1H).

Yield: 77%

### Example 29

### 2-(4-(1-(((4-Fluorobenzyl)oxy)imino)-2-phenylethyl)phenoxy)-1-morpholinoethanone

¹H NMR: 3.56-3.63 (m, 81H), 4.11 (s, 2H), 4.67 (s, 2H), 5.19 (s, 2H), 6.87 (d, J=8.8 Mz, 2H), 7.01 (t, J=8.7 Hz, 2H), 7.14-7.22 (m, 5H), 7.29-7.32 (m, 2H), 7.58 (d, J=8.8 Mz, 2H).

Yield: 91%

### Example 30

### 2-(4-(1-(((4-Fluorobenzyl)oxy)imino)-2-phenylethyl)phenoxy)-1-(4-methylpiperazin-1-yl)ethanone

¹H NMR (DMSO-d₆): 2.16 (s, 3H), 2.23 (m, 2H), 2.29 (m, 2H), 3.40 (m, 4H), 4.12 (s, 2H), 4.80 (s, 2H), 5.19 (s, 2H), 6.86 (d, J=6.8 Hz, 2H), 7.12-7.22 (m, 7H), 7.40-7.43 (dd, J=8.4 & 8.4 Hz, 2H), 7.58 (d, J=6.8 Hz, 2H).

Yield: 94%

### Example 31

### 2-(4-(1-(((4-Fluorobenzyl)oxy)imino)-2-phenylethyl)phenoxy)-N-(2-morpholino-2-oxoethyl)acetamide

¹H NMR (DMSO-d₆): 3.40-3.41 (m, 4H), 3.52-3.54 (m, 4H), 3.99 (d, J=5.2 Hz, 2H), 4.13 (s, 2H), 4.54 (s, 2H), 5.19 (s, 2H), 6.94 (d, J=8.8 Mz, 2H), 7.12-7.22 (m, 7H), 7.40-7.43 (m, 2H), 7.61 (d, J=8.8 Hz, 2H), 8.08 (t, J=5.2 Mz, 1H).

Yield: 38%

### Example 32

### 2-(4-(1-(((4-Chlorobenzyl)oxy)imino)-2-phenylethyl)-2-methylphenoxy)-1-morpholinoethanone

¹H NMR (DMSO-d₆): 2.14 (s, 3H), 3.42 (bs, 4H), 3.53-3.57 (m, 4H), 4.12 (s, 2H), 4.84 (s, 2H), 5.19 (s, 2H), 6.78 (d, J=8.8 Hz, 1H), 7.12-7.14 (m, 3H), 7.19-7.23 (m, 2H), 7.36-7.42 (m, 5H), 7.49 (d, J=1.2 Hz, 1H).

Yield: 23%

### Example 33

### 2-(4-(1-(((4-Chlorobenzyl)oxy)imino)-2-phenylethyl)phenoxy)-1-morpholinoethanone

¹H NMR (DMSO-d₆): 3.41 (bs, 4H), 3.53-3.57 (m, 4H), 4.13 (s, 2H), 4.82 (s, 2H), 5.20 (s, 2H), 6.86 (d, J=8.8 Hz, 2H), 7.13-7.15 (m, 3H), 7.19-7.23 (m, 2H), 7.37-7.42 (m, 4H), 7.56 (d, J=8.8 Hz, 2H).

Yield: 68%

### Example 34

### 2-(2-methyl-4-(2-phenyl-1-(((4-(trifluoromethoxy)benzyl)oxy)imino)ethyl)phenoxy)-1-morpholinoethanone

¹H NMR: 2.20 (s, 3H), 3.60-3.645 (m, 8H), 4.12 (s, 2H), 4.68 (s, 2H), 5.22 (s, 2H), 6.77 (d, J=8.4 Mz, 1H), 7.15-7.24 (m, 7H), 7.33 (d, J=8.4 Mz, 2H), 7.37-7.39 (m, 1H), 7.51 (d, J=6.8 Hz, 1H).

Yield: 87%

### Example 35

### 1-morpholino-2-(4-(2-phenyl-1-(((4-(trifluoromethoxy)benzyl)oxy)imino)ethyl)phenoxy)ethanone

¹H NMR (DMSO-d₆): 3.41 (bs, 4H), 3.53-3.57 (m, 4H), 4.14 (s, 2H), 4.82 (s, 2H), 5.24 (s, 2H), 6.86 (d, J=9.2 Hz, 2H), 7.11-7.15 (m, 3H), 7.19-7.22 (m, 2H), 7.33 (d, J=8.0 Hz, 2H). 7.48 (d, J=8.8 Hz, 2H), 7.57 (d, J=8.8 Hz, 2H).

Yield: 48%

### Example 36

### 2-(4-(1-(((4-methoxybenzyl)oxy)imino)-2-phenylethyl)-2-methylphenoxy)-1-morpholinoethanone

¹H NMR (DMSO-d₆): 2.14 (s, 3H), 3.42 (bs, 4H), 3.52-3.57 (m, 4H), 3.73 (s, 3H), 4.09 (s, 2H), 4.83 (s, 2H), 5.12 (s, 2H), 6.78 (d, J=8.8 Hz, 11), 6.89-6.92 (m, 2H), 7.10-7.14 (m, 3H), 7.18-7.21 (m, 2H), 7.29 (dd, J=11.4 & 2.6 Hz, 2H), 7.38 (dd, J=8.6 & 2.2 Hz, 1H), 7.50 (d, J=2.0 Hz, 1H).

Yield: 96%

### Example 37

### 2-(4-(1-(((4-Methoxybenzyl)oxy)imino)-2-phenylethyl)phenoxy)-1-morpholinoethanone

¹H NMR (DMSO-d₆): 3.41 (bs, 4H), 3.52-3.57 (m, 4H), 3.73 (s, 3H), 4.10 (s, 2H), 4.82 (s, 2H), 5.13 (s, 2H), 6.85-6.92 (comp, 4H), 7.10-7.14 (m, 3H), 7.18-7.21 (m, 2H), 7.30-7.33 (m, 2H), 7.56-7.59 (m, 2H).

Yield: 88%

### Example 38

### 2-(4-(1-(((4-methoxybenzyl)oxy)imino)-2-phenylethyl)-2-methylphenoxy)-1-(4-methylpiperazin-1-yl)ethanone

¹H NMR (CD₃OD): 2.21 (s, 3H), 2.29 (s, 3H), 2.40-2.46 (m, 4H), 3.59-3.60 (m, 4H), 3.78 (s, 3H), 4.10 (s, 2H), 4.81 (s, 2H), 5.13 (s, 2H), 6.76 (d, J=8.8 Hz, 1H), 6.87 (dd, J=6.8 & 2.0 Hz, 2H), 7.10-7.12 (m, 3H), 7.14-7.18 (m, 2H), 7.27 (dd, J=6.8 & 2.0 Hz, 2H), 7.38 (dd, J=8.4 & 2.0 Hz, 1H), 7.48 (d, J=1.6 Hz, 1H).

Yield: 33%

### Example 39

### 2-(4-(1-(((4-methoxybenzyl)oxy)imino)-2-phenylethyl)phenoxy)-1-(4-methylpiperazin-1-yl)ethanone

¹H NMR (CD₃OD): 2.69 (s, 3H), 2.97-3.01 (m, 4H), 3.75 (bs, 4H), 3.78 (s, 3H), 4.12 (s, 2H), 4.83 (s, 2H), 5.14 (s, 2H), 6.87-6.92 (comp, 4H), 7.11-7.18 (m, 5H), 7.28 (d, J=8.8 Hz, 2H), 7.58 (d, J=6.8 & 2.0 Hz, 2H).

Yield: 16%

### Example 40

### 2-(4-(1-(((4-methoxybenzyl)oxy)imino)-2-phenylethyl)-2-methylphenoxy)-1-(piperidin-1-yl)ethanone

¹H NMR (DMSO-d₆): 1.40 (bs, 2H), 1.48-1.49 (m, 2H), 1.53-1.56 (m, 2H), 2.14 (s, 3H), 3.37-3.39 (m, 4H), 3.73 (s, 3H), 4.08 (s, 2H), 4.79 (s, 2H), 5.12 (s, 2H), 6.76 (d, J=8.4 Hz, 1H), 6.89-6.92 (m, 2H), 7.10-7.14 (m, 3H), 7.17-7.21 (m, 2H), 7.29-7.32 (m, 2H), 7.38 (dd, J=8.6 & 2.2 Hz, 1H), 7.49 (d, J=2.0 Hz, 1H).

Yield: 80%

### Example 41

### 2-(4-(1-(((4-methoxybenzyl)oxy)imino)-2-phenylethyl)phenoxy)-1-(piperidin-1-yl)ethanone

¹H NMR (DMSO-d₆): 1.40 (bs, 2H), 1.49-1.50 (m, 2H), 1.53-1.56 (m, 2H), 3.33-3.39 (m, 4H), 3.73 (s, 3H), 4.09 (s, 2H), 4.77 (s, 2H), 5.13 (s, 2H), 6.84-6.92 (comp, 4H), 7.10-7.14 (m, 3H), 7.18-7.21 (m, 2H), 7.30-7.33 (m, 2H), 7.5-7.59 (m, 2H).

Yield: 80%

### Example 42

### 2-(4-(1-(((4-(methylsulfonyl)benzyl)oxy)imino)propyl)phenoxy)-1-morpholinoethanone

¹H NMR: 1.04 (t, J=7.6 Hz, 3H), 2.72 (q, J=7.6 Hz, 2H), 3.20 (s, 3H), 3.44 (bs, 4H), 3.56-3.60 (m, 4H), 4.86 (s, 2H), 5.27 (s, 2H), 6.91 (d, J=9.2 Hz, 2H), 7.54 (d, J=8.8 Hz, 2H), 7.62 (d, J=8.4 Hz, 2H), 7.91 (d, J=8.4 Hz, 2H).

Yield: 44%

### Example 43

### 1-(4-methylpiperazin-1-yl)-2-(4-(1-(((4-(methylsulfonyl)benzyl)oxy)imino)propyl)phenoxy)ethanone

¹H NMR: 1.12 (t, J=7.6 Hz, 3H), 2.28 (s, 3H), 2.36-2.40 (m, 4H), 2.74 (q, J=7.6 Hz, 2H), 3.05 (s, 3H), 3.56-3.64 (m, 4H), 4.70 (s, 2H), 5.27 (s, 2H), 6.91-6.94 (m, 2H), 7.53-7.58 (m, 4H), 7.92 (dd, J=6.8 & 1.6 Hz, 2H).

Yield: 35%

### Example 44

### 1-morpholino-2-(4-(2-phenyl-1-((pyridin-2-ylmethoxy)imino)ethyl)phenoxy)ethanone

¹H NMR: 3.55-3.62 (m, 8H), 4.20 (s, 2H), 4.66 (S, 2H), 5.38 (s, 2H), 6.85-6.89 (m, 2H), 7.15-7.21 (m, 2H), 7.25-7.27 (m, 5H), 7.58-7.63 (m, 3H), 8.56 (d, J=4.4 Hz, 1H).

Yield: 70%

### Example 45

### 2-(4-(1-((2-(1H-indol-1-yl)ethoxy)imino)propyl)phenoxy)-1-morpholinoethanone

¹H NMR: 1.00 (t, J=7.6 Hz, 3H), 2.59 (q, J=7.6 Hz, 2H), 3.60-3.68 (m, 8H), 4.47 (s, 4H), 4.71 (s, 2H), 6.50 (d, J=2.8 Hz, 1H), 6.93 (d, J=8.8 Hz, 2H), 7.07-7.12 (m, 2H), 7.19 (t, J=7.6 Hz, 1H), 7.37 (d, J=8.4 Hz, 1H), 7.55 (d, J=8.8 Hz, 2H), 7.61 (d, J=7.6 Hz, 11H).

Yield: 84%

### Example 46

### 2-(4-(1-((2-(1H-indol-1-yl)ethoxy)imino)propyl)phenoxy)-1-(4-methylpiperazin-1-yl)ethanone

¹H NMR: 1.00 (t, J=7.6 Hz, 3H), 2.29 (s, 3H), 2.37-2.42 (m, 4H), 2.59 (q, J=7.4 Hz, 2H), 3.59 (t, J=4.8 Hz, 2H), 3.64 (t, J=4.8 Hz, 2H), 4.46 (s, 4H), 4.71 (s, 2H), 6.49 (d, J=3.2 Hz, 1H), 6.93 (d, J=9.2 Hz, 2H), 7.07-7.12 (m, 2H), 7.19 (t, J=7.6 Hz, 1H), 7.37 (d, J=8.4 Hz, 1H), 7.55 (d, J=8.8 Hz, 2H), 7.61 (d, J=8.0 Hz, 1H).

Yield: 82%

### Example 47

### 3-methyl-2-((((1-(4-(2-morpholino-2-oxoethoxy)phenyl)propylidene)amino)oxy)methyl)quinazolin-4(3H)-one

¹H NMR (DMSO-d₆): 1.08 (t, J=7.4 Hz, 3H), 2.72 (q, J=7.6 Hz, 2H), 3.42-3.43 (m, 4H), 3.54-3.58 (m, 4H), 3.62 (s, 3H), 4.84 (s, 2H), 5.32 (s, 2H), 6.90 (d, J=8.8 Hz, 2H), 7.51-7.56 (m, 3H), 7.66 (d, J=8.0 Hz, 1H), 7.80-7.84 (m, 1H), 8.12 (d, J=7.2 Hz, 1H).

Yield: 47%

### Example 48

### 2-(4-(1-(((5-ethylpyrimidin-2-yl)oxy)imino)propyl)phenoxy)-1-morpholinoethanone

¹H NMR (DMSO-d₆): 1.12 (t, J=7.6 Hz, 3H), 1.20 (t, J=6.8 Hz, 3H), 2.56-2.61 (q, 2H), 2.86-2.92 (q, 2H), 3.45 (br s, 4H), 3.55-3.61 (m, 4H), 4.90 (s, 2H), 6.98-7.02 (m, 2H), 7.70-7.73 (m, 2H), 8.54 (s, 2H).

Yield: 86%

### Example 49

### 2-(4-(1-(((5-ethylpyrimidin-2-yl)oxy)imino)propyl)phenoxy)-1-(4-methylpiperazin-1-yl)ethanone

¹H NMR (DMSO-d₆): 1.12 (t, J=7.6 Hz, 3H), 1.18 (t, J=7.6 Hz, 3H), 2.17 (s, 3H), 2.25-2.33 (m, 4H), 2.56-2.61 (q, 2H), 2.86-2.92 (q, 2H), 3.44 (t, J=5.0 Hz, 4H), 4.88 (s, 2H), 6.97-7.01 (m, 2H), 7.70-7.73 (m, 2H), 8.54 (s, 2H).

Yield: 72%

### Example 50

### 2-(4-(1-(((5-methyl-2-phenyloxazol-4-yl)methoxy)imino)propyl)phenoxy)-1-(4-methylpiperazin-1-yl)ethanone

¹H NMR: 1.07 (t, J=7.4 Hz, 3H), 2.28 (s, 3H), 2.36-2.42 (m, 4H), 2.47 (s, 3H), 2.70 (q, J=7.6 Hz, 2H), 3.57-3.65 (m, 4H), 4.70 (s, 2H), 5.11 (s, 2H), 6.91-6.94 (m, 2H), 7.38-7.45 (m, 3H), 7.56-7.60 (m, 2H), 7.99-8.02 (m, 2H).

Yield: 80%

### Example 51

### 2-(4-(1-(((5-methyl-2-phenyloxazol-4-yl)methoxy)imino)propyl)phenoxy)-1-morpholinoethanone

¹H NMR: 1.07 (t, J=7.6 Hz, 3H), 2.47 (s, 3H), 2.70 (q, J=7.6 Hz, 2H), 3.60-3.67 (m, 8H), 4.71 (s, 2H), 5.11 (s, 2H), 6.91-6.94 (m, 2H), 7.40-7.45 (m, 3H), 7.56-7.60 (m, 2H), 7.99-8.02 (m, 2H).

Yield: 85%

### Example 52

### 2-(4-(1-(((3-(tert-butyl)-1-(p-tolyl)-1H-pyrazol-5-yl)methoxy)imino)propyl)phenoxy)-1-morpholinoethanone

¹H NMR: 1.06 (t, J=7.6 Hz, 3H), 1.36 (s, 9H), 2.37 (s, 3H), 2.68 (q, J=7.6 Hz, 2H), 3.60-3.67 (m, 8H), 4.71 (s, 2H), 5.11 (s, 2H), 6.37 (s, 1H), 6.92 (d, J=9.2 Hz, 2H), 7.21 (d, J=8.0 Hz, 2H), 7.44 (d, J=8.4 Hz, 2H), 7.55 (dd, J=6.8 & 2.0 Hz, 2H).

Yield: 63%

### Example 53

### 2-(4-(1-(((3-(tert-butyl)-1-(p-tolyl)-1H-pyrazol-5-yl)methoxy)imino)propyl)phenoxy)-1-(4-methylpiperazin-1-yl)ethanone

¹H NMR: 1.06 (t, J=7.4 Hz, 3H), 1.36 (s, 9H), 2.29 (s, 3H), 2.37-2.41 (m, 7H), 2.69 (q, J=7.6 Hz, 2H), 3.58-3.65 (m, 4H), 4.70 (s, 2H), 5.11 (s, 2H), 6.37 (s, 1H), 6.92 (dd, J=7.2 & 2.0 Hz, 2H), 7.21 (d, J=8.0 Hz, 2H), 7.44 (d, J=8.4 Hz, 2H), 7.55 (dd, J=6.8 & 2.0 Hz, 2H).

Yield: 57%

### Example 54

### 1-(piperidin-1-yl)-2-((5-(((4-(trifluoromethyl)benzyl)oxy)imino)-5,6,7,8-tetrahydronaphthalen-2-yl)oxy)ethanone

¹H NMR (DMSO-d₆): 1.42 (s, 2H), 1.52-1.58 (m, 4H), 1.72-1.78 (m, 2H), 2.66-2.73 (m, 4H), 3.39 (t, J=6.4 Hz, 4H), 4.78 (s, 2H), 5.25 (s, 2H), 6.76 (t, J=5.6 Hz, 2H), 7.59 (d, J=8.0 Hz, 2H), 7.71 (t, J=9.0 Hz, 3H).

Yield: 31%

### Example 55

### 1-morpholino-2-((5-(((4-(trifluoromethyl)benzyl)oxy)imino)-5,6,7,8-tetrahydronaphthalen-2-yl)oxy)ethanone

¹H NMR (DMSO-d₆): 1.74 (t, J=5.8 Hz, 2H), 2.65-2.72 (m, 4H), 3.43 (bs, 4H), 3.55 (bd, 4H), 4.82 (s, 2H), 5.24 (s, 2H), 6.74 (d, J=8.0 Hz, 2H), 7.58 (d, J=8.0 Hz, 2H), 7.68-7.73 (m, 3H).

Yield: 12%

### Example 56

### 1-(4-methylpiperazin-1-yl)-2-((5-(((4-(trifluoromethyl)benzyl)oxy)imino)-5,6,7,8-tetrahydronaphthalen-2-yl)oxy)ethanone

¹H NMR (DMSO-d₆): 1.75 (t, J=5.8 Hz, 2H), 2.18 (s, 3H), 2.25 (d, J=25.2 Hz, 4H), 2.66-2.73 (m, 4H), 3.59 (s, 4H), 4.81 (s, 2H), 5.25 (s, 2H), 6.74 (d, J=8.0 Hz, 2H), 7.59 (d, J=8.0 Hz, 2H), 7.69-7.79 (m, 3H).

Yield: 50%

### Example 57

### N-morpholino-2-((5-(((4-(trifluoromethyl)benzyl)oxy)imino)-5,6,7,8-tetrahydronaphthalen-2-yl)oxy)acetamide

¹H NMR (DMSO-d₆): 1.81-1.88 (m, 2H), 2.70-2.79 (m, 4H), 2.85 (t, J=4.4 Hz, 4H), 3.82 (t, J=4.6 Hz, 4H), 4.52 (s, 2H), 5.25 (s, 2H), 6.65 (d, J=2.4 Hz, 1H), 6.74 (dd, J=8.8 & 2.8 Hz, 1H), 7.24 (d, J=7.2 Hz, 1H), 7.49 (d, J=8.0 Hz, 2H), 7.60 (d, J=8.0 Hz, 2H), 7.90 (d, J=8.8 Hz, 1H).

Yield: 28%

### Example 58

### N-(piperidin-1-yl)-2-((5-(((4-(trifluoromethyl)benzyl)oxy)imino)-5,6,7,8-tetrahydronaphthalen-2-yl)oxy)acetamide

¹H NMR (DMSO-d₆): 1.33 (brs, 2H), 1.54 (brs, 4H), 2.69 (brs, 8H), 1.75 (brs, 2H), 4.41 (s, 1H), 4.82 (s, 1H), 5.25 (s, 2H), 6.64-6.79 (m, 2H), 7.59 (d, J=7.6 Hz, 2H), 7.72 (t, J=9.6 Hz, 3H), 8.74 (s, 1H).

Yield: 11%

### Example 59

### N-(5-chlorobenzo[d]oxazol-2-yl)-2-((5-(((4-(trifluoromethyl)benzyl)oxy)imino)-5,6,7,8-tetrahydronaphthalen-2-yl)oxy)acetamide

¹H NMR (DMSO-d₆): 1.77 (t, J=5.8 Hz, 2H), 2.67-2.73 (m, 4H), 4.97 (s, 2H), 5.25 (s, 2H), 6.79-6.84 (m, 2H), 7.29 (dd, J=8.4 & 2.0 Hz, 1H), 7.59 (d, J=8.0 Hz, 2H), 7.65-7.68 (m, 2H), 7.72 (dd, J=8.8 & 2.4 Hz, 3H), 12.09 (s, 1H).

Yield: 40%

### Example 60

### N-(4-methylpyrimidin-2-yl)-2-((5-(((4-(trifluoromethyl)benzyl)oxy)imino)-5,6,7,8-tetrahydronaphthalen-2-yl)oxy)acetamide

¹H NMR (DMSO-d₆): 1.75 (t, J=5.8 Hz, 2H), 2.41 (s, 3H), 2.66-2.73 (m, 4H), 5.0 (s, 2H), 5.25 (s, 2H), 6.73-6.79 (m, 2H), 7.07 (d, J=5.2 Hz, 1H), 7.59 (d, J=8.0 Hz, 2H), 7.71 (dd, J=8.8 & 2.8 Hz, 3H), 8.49 (d, J=5.2 Hz, 1H), 10.62 (s, 1H).

Yield: 30%

### Example 61

### N-(1,3-dimethyl-1H-pyrazol-5-yl)-2-((5-(((4-(trifluoromethyl)benzyl)oxy)imino)-5,6,7,8-tetrahydronaphthalen-2-yl)oxy)acetamide

¹H NMR (DMSO-d₆): 1.75 (s, 2H), 2.07 (s, 3H), 2.72-2.69 (m, 4H), 3.53 (s, 3H), 4.74 (s, 2H), 5.25 (s, 2H), 5.95 (s, 1H), 6.85-6.80 (m, 2H), 7.59 (d, J=8.0 Hz, 2H), 7.71-7.75 (m, 3H), 10.05 (s, 1H).

Yield: 40%

### Example 62

### tert-butyl 4-((2-((5-(((4-(trifluoromethyl)benzyl)oxy)imino)-5,6,7,8-tetrahydronaphthalen-2-yl)oxy)acetamido)oxy)piperidine-1-carboxylate

¹H NMR (DMSO-d₆): 1.38 (s, 9H), 1.44 (t, J=4.2 Hz, 2H), 1.75 (t, J=5.8 Hz, 4H), 2.66-2.73 (m, 4H), 3.07 (s, 2H), 3.57 (t, J=6.2 Hz, 2H), 3.94 (s, 1H), 4.50 (s, 2H), 5.25 (s, 2H), 6.76 (s, 1H), 6.78 (d, J=8.8 Hz, 1H), 7.59 (d, J=8.0 Hz, 2H), 7.72 (d, J=7.6 Hz, 3H), 11.25 (s, 1H).

Yield: 20%

### Example 63

### N-(2-isocyanophenyl)-2-((5-(((4-(trifluoromethyl)benzyl)oxy)imino)-5,6,7,8-tetrahydronaphthalen-2-yl)oxy)acetamide

¹H NMR (DMSO-d₆): 1.76 (s, 2H), 2.71 (d, J=6.0 Hz, 4H), 4.79 (s, 2H), 5.26 (s, 2H), 6.86 (t, J=7.4 Hz, 2H), 7.39 (t, J=7.8 Hz, 1H), 7.62 (t, J=10.4 Hz, 3H), 7.69 (q, J=9.4 Hz, 4H), 7.83 (d, J=8.0 Hz, 1H), 10.28 (s, 1H).

Yield: 57%

The following compounds can be prepared by procedure similar to those described above with appropriate variations of reactions, reaction conditions and quantities of reagents.

### Example 64

### 2-((4-((Naphthalen-2-ylmethoxy)imino)chroman-7-yl)oxy)-1-(piperidin-1-yl)ethanone

### Example 65

### 2-((4-((Benzyloxy)imino)chroman-7-yl)oxy)-1-(piperidin-1-yl)ethanone

### Example 66

### 2-((4-(((4-Fluorobenzyl)oxy)imino)chroman-7-yl)oxy)-1-(piperidin-1-yl)ethanone

### Example 67

### 1-(Piperidin-1-yl)-2-((4-(((4-(trifluoromethyl)benzyl)oxy)imino)chroman-7-yl)oxy)ethanone

### Example 68

### 2-((4-(((4-Methoxybenzyl)oxy)imino)chroman-7-yl)oxy)-1-(piperidin-1-yl)ethanone

### Example 69

### 2-((4-(((4-Methoxybenzyl)oxy)imino)chroman-7-yl)oxy)-1-morpholinoethanone

### Example 70

### 2-((4-(((4-Methoxybenzyl)oxy)imino)chroman-7-yl)oxy)-1-(4-methylpiperazin-1-yl)ethanone

### Example 71

### 8-(2-((5-(((4-(Trifluoromethyl)benzyl)oxy)imino)-5,6,7,8-tetrahydronaphthalen-2-yl)oxy)acetyl)-1-oxa-3,8-diazaspiro[4.5]decan-2-one

### Example 72

### 8-(2-((4-(((4-(Trifluoromethyl)benzyl)oxy)imino)chroman-7-yl)oxy)acetyl)-1-oxa-3,8-diazaspiro[4.5]decan-2-one

### Example 73

### 8-(2-((4-(((4-Methoxybenzyl)oxy)imino)chroman-7-yl)oxy)acetyl)-1-oxa-3,8-diazaspiro[4.5]decan-2-one

### Example 74

### 8-(2-((4-((Naphthalen-2-ylmethoxy)imino)chroman-7-yl)oxy)acetyl)-1-oxa-3,8-diazaspiro[4.5]decan-2-one

### Example 75

### 8-(2-((4-(((6-(Trifluoromethyl)pyridin-3-yl)methoxy)imino)chroman-7-yl)oxy)acetyl)-1-oxa-3,8-diazaspiro[4.5]decan-2-one

### Example 76

### 8-(2-((4-(((1-Methyl-1H-indol-6-yl)methoxy)imino)chroman-7-yl)oxy)acetyl)-1-oxa-3,8-diazaspiro[4.5]decan-2-one

### Example 77

### 8-(2-(4-(1-(((4-Methoxybenzyl)oxy)imino)propyl)phenoxy)acetyl)-1-oxa-3,8-diazaspiro[4.5]decan-2-one

### Example 78

### 8-(2-(4-(1-(((4-(Trifluoromethyl)benzyl)oxy)imino)propyl)phenoxy)acetyl)-1-oxa-3,8-diazaspiro[4.5]decan-2-one

### Example 79

### 8-(2-(4-(1-(((4-(Trifluoromethyl)benzyl)oxy)imino)ethyl)phenoxy)acetyl)-1-oxa-3,8-diazaspiro[4.5]decan-2-one

### Example 80

### 8-(2-(4-(1-(((4-Methoxybenzyl)oxy)imino)ethyl)phenoxy)acetyl)-1-oxa-3,8-diazaspiro[4.5]decan-2-one

### Example 81

### 8-(2-(4-(1-((4-Methoxyphenoxy)imino)ethyl)phenoxy)acetyl)-1-oxa-3,8-diazaspiro[4.5]decan-2-one

### Example 82

### 8-(2-(4-(1-((4-(Trifluoromethyl)phenoxy)imino)ethyl)phenoxy)acetyl)-1-oxa-3,8-diazaspiro[4.5]decan-2-one

### Example 83

### 1-(6-Azaspiro[2.5]octan-6-yl)-2-(4-(1-(((4-(trifluoromethyl)benzyl)oxy)imino)ethyl)phenoxy)ethanone

### Example 84

### 1-(6-Azaspiro[2.5]octan-6-yl)-2-(4-(1-(((4-(trifluoromethyl)benzyl)oxy)imino)propyl)phenoxy)ethanone

### Example 85

### 2-(4-(2-Phenyl-1-(((4-(trifluoromethyl)benzyl)oxy)imino)ethyl)phenoxy)-1-(6-azaspiro[2.5]octan-6-yl)ethanone

### Example 86

### 2-(4-(1-(((4-Methoxybenzyl)oxy)imino)propyl)phenoxy)-1-(6-azaspiro[2.5]octan-6-yl)ethanone

### Example 87

### 2-(4-(1-(((4-Fluorobenzyl)oxy)imino)propyl)phenoxy)-1-(6-azaspiro[2.5]octan-6-yl)ethanone

### Example 88

### 2-(4-(1-((3-(6-Azaspiro[2.5]octan-6-yl)propoxy)imino)propyl)phenoxy)-1-(piperidin-1-yl)ethanone

### Example 89

### 2-(4-(1-((2-(6-Azaspiro[2.5]octan-6-yl)ethoxy)imino)propyl)phenoxy)-1-(piperidin-1-yl)ethanone

### Example 90

### 2-(4-(1-((2-(Methyl(phenyl)amino)ethoxy)imino)propyl)phenoxy)-1-(piperidin-1-yl)ethanone

### Example 91

### 2-(4-(1-((2-((4-Fluorophenyl)(methyl)amino)ethoxy)imino)propyl)phenoxy)-1-(piperidin-1-yl)ethanone

### Example 92

### 2-(4-(1-((2-((4-Fluorophenyl)(methyl)amino)ethoxy)imino)propyl)phenoxy)-1-morpholinoethanone

### Example 93

### 2-(4-(1-((2-((4-Fluorophenyl)(methyl)amino)ethoxy)imino)propyl)phenoxy)-1-thiomorpholinoethanone

### Example 94

### 2-(4-(1-((Naphthalen-2-ylmethoxy)imino)propyl)phenoxy)-1-(6-azaspiro[2.5]octan-6-yl)ethanone

### Example 95

### 2-(4-(1-((Naphthalen-2-ylmethoxy)imino)pentyl)phenoxy)-1-(6-azaspiro[2.5]octan-6-yl)ethanone

### Example 96

### 2-(4-(1-(((3,4-Dimethylbenzyl)oxy)imino)pentyl)phenoxy)-1-(6-azaspiro[2.5]octan-6-yl)ethanone

### Example 97

### 2-(4-(1-(((3,4-Dimethylbenzyl)oxy)imino)-3-methoxypropyl)phenoxy)-1-(6-azaspiro[2.5]octan-6-yl)ethanone

### Example 98

### 2-(4-(1-((Dibenzo[b,d]thiophen-3-ylmethoxy)imino)-3-methoxypropyl)phenoxy)-1-(6-azaspiro[2.5]octan-6-yl)ethanone

### Example 99

### 2-(4-(1-((Dibenzo[b,d]thiophen-3-ylmethoxy)imino)-3-methoxypropyl)phenoxy)-1-(piperidin-1-yl)ethanone

### Example 100

### N-((1-methylpiperidin-4-yl)oxy)-2-(4-(2-morpholino-1-(((4-(trifluoromethyl)benzyl)oxy)imino)ethyl)phenoxy)ethanethioamide

### Example 101

### 2-(4-(1-(((3,5-Bis(trifluoromethyl)benzyl)oxy)imino)-2-morpholinoethyl)phenoxy)-N-((1-methylpiperidin-4-yl)oxy)ethanethioamide

### Example 102

### 2-(4-(1-(((3,5-Bis(trifluoromethyl)benzyl)oxy)imino)-2-morpholinoethyl)phenoxy)-N-((1-methylpiperidin-4-yl)oxy)acetamide

### Example 103

### 2-(4-(1-(((3,5-Bis(trifluoromethyl)benzyl)oxy)imino)-2-(piperidin-1-yl)ethyl)phenoxy)-2-methyl-N-((1-methylpiperidin-4-yl)methyl)propanamide

### Example 104

### 2-Methyl-N-((1-methylpiperidin-4-yl)methyl)-2-(4-(1-(((tetrahydro-2H-pyran-4-yl)methoxy)imino)ethyl)phenoxy)propanamide

### Example 105

### N-((1-methylpiperidin-4-yl)methyl)-2-(4-(1-(((tetrahydro-2H-pyran-4-yl) methoxy)imino)ethyl)phenoxy)propanamide

### Example 106

### 2-(4-(2-Methoxy-1-(((4-(trifluoromethyl)benzyl)oxy)imino)ethyl)phenoxy)-N-((1-methylpiperidin-4-yl)methyl)ethanethioamide

### Example 107

### 2-(4-(2-Methoxy-1-(((4-(trifluoromethyl)benzyl)oxy)imino)ethyl)phenoxy)-1-(piperidin-1-yl)ethanethione

### Example 108

### 2-(4-(1-(([1,1′-Biphenyl]-4-ylmethoxy)imino)-2-methoxyethyl)phenoxy)-1-(piperidin-1-yl)ethanethione

### Example 109

### 2-(4-(2-Methoxy-1-(((4′-(trifluoromethyl)-[1,1′-biphenyl]-4-yl)methoxy)imino)ethyl)phenoxy)-1-(piperidin-1-yl)ethanethione

### Example 110

### 2-(4-(2-Methoxy-1-(((4′-(trifluoromethyl)-[1,1′-biphenyl]-4-yl)methoxy)imino)ethyl)phenoxy)-1-(4-methylpiperazin-1-yl)ethanethione

### Example 111

### N-(cyclohexylmethyl)-3-(4-(1-(((4-(trifluoromethyl)benzyl)oxy)imino)propyl)phenyl)propanethioamide

### Example 112

### N-(cyclohexylmethyl)-3-(4-(2-phenyl-1-(((4-(trifluoromethyl)benzyl)oxy)imino)ethyl)phenyl)propanethioamide

### Example 113

### N-((1-methylpiperidin-4-yl)methyl)-2-(4-(2-phenyl-1-(((4-(trifluoromethyl)benzyl)oxy)imino)ethyl)phenoxy)ethanethioamide

### Example 114

### 2-(4-(2-Cyclohexyl-1-(((4-(trifluoromethyl)benzyl)oxy)imino)ethyl)phenoxy)-N-((1-methylpiperidin-4-yl)methyl)ethanethioamide

### Example 115

### N-((1-methylpiperidin-4-yl)methyl)-2-(4-(2-morpholino-1-(((4-(trifluoromethyl)benzyl)oxy)imino)ethyl)phenoxy)ethanethioamide

### Example 116

### 1-(4-Allylpiperazin-1-yl)-2-(4-(1-((pyridin-4-ylmethoxy)imino)propyl)phenoxy)ethanone

### Example 117

### 1-(4-Allylpiperazin-1-yl)-2,2-difluoro-2-(4-(1-((pyridin-4-ylmethoxy)imino)propyl)phenoxy)ethanone

### Example 118

### N-(cyclohexylmethyl)-2,2-difluoro-N-methyl-2-(4-(1-((pyridin-4-ylmethoxy)imino)propyl)phenoxy)acetamide

### Example 119

### N-(cyclohexylmethyl)-2,2-difluoro-2-(4-(1-((pyridin-4-ylmethoxy)imino)propyl) phenoxy)acetamide

### Example 120

### 2-(4-(1-((Benzyloxy)imino)propyl)phenoxy)-N-(cyclohexylmethyl)-2,2-difluoroacetamide

### Example 121

### N-(cyclohexylmethyl)-2,2-difluoro-2-(4-(1-(((4-(trifluoromethyl)benzyl)oxy)imino)propyl)phenoxy)acetamide

### Example 122

### N-(cyclohexylmethyl)-2-(4-(1-(((4-(trifluoromethyl)benzyl)oxy)imino)propyl)phenoxy)ethanethioamide

### Example 123

### 2-(4-(1-(((1,2,3,4-Tetrahydronaphthalen-1-yl)oxy)imino)propyl)phenoxy)-1-(4-(3-(trifluoromethyl)phenyl)piperazin-1-yl)ethanone

### Example 124

### 2-(4-(1-((Cyclohexyloxy)imino)propyl)phenoxy)-1-(4-(3-(trifluoromethyl)phenyl)piperazin-1-yl)ethanone

### Example 125

### 2-(4-(1-((Benzyloxy)imino)propyl)phenoxy)-1-(4-(3-(trifluoromethyl)phenyl)piperazin-1-yl)ethanone

### Example 126

### 2-(4-(1-(((2-Fluorobenzyl)oxy)imino)propyl)phenoxy)-1-(4-(3-(trifluoromethyl)phenyl)piperazin-1-yl)ethanone

### Example 127

### 2-(4-(1-((Pyridin-4-ylmethoxy)imino)propyl)phenoxy)-1-(4-(3-(trifluoromethyl)phenyl)piperazin-1-yl)ethanone

### Example 128

### 1-(4-Phenylpiperazin-1-yl)-2-(4-(1-((pyridin-4-ylmethoxy)imino)propyl)phenoxy)ethanone

### Example 129

### 8-(2-(4-(1-(((1,2,3,4-Tetrahydronaphthalen-1-yl)oxy)imino)propyl)phenoxy)acetyl)-1-oxa-3,8-diazaspiro[4.5]decan-2-one

### Example 130

### 1-(Piperidin-1-yl)-2-(4-(1-(((1,2,3,4-tetrahydronaphthalen-1-yl)oxy)imino)propyl)phenoxy)ethanone

### Example 131

### 1-(4-(2-Hydroxyethyl)piperazin-1-yl)-2-(4-(1-(((1,2,3,4-tetrahydronaphthalen-1-yl)oxy)imino)propyl)phenoxy)ethanone

### Example 132

### 2-Hydroxy-1-(4-(2-(4-(1-(((1,2,3,4-tetrahydronaphthalen-1-yl)oxy)imino)propyl)phenoxy)acetyl)piperazin-1-yl)ethanone

### Example 133

### 2-Hydroxy-1-(4-(2-(4-(1-(((2-methylbenzyl)oxy)imino)propyl)phenoxy)acetyl)piperazin-1-yl)ethanone

### Example 134

### 2-Hydroxy-1-(4-(2-(4-(1-(((2-(trifluoromethyl)benzyl)oxy)imino)propyl)phenoxy)acetyl)piperazin-1-yl)ethanone

### Example 135

### 2-(4-(1-(((5-Fluoro-2-(trifluoromethyl)benzyl)oxy)imino)propyl)phenoxy)-1-(4-(2-hydroxyacetyl)piperazin-1-yl)ethanone

### Example 136

### N-Methyl-2-((1-methyl-5-((phenoxyimino)methyl)-1H-indol-2-yl)oxy)-N-(2-oxo-2-(piperidin-1-yl)ethyl)acetamide

### Example 137

### 2-((5-(((Benzyloxy)imino)methyl)-1-methyl-1H-indol-2-yl)oxy)-N-methyl-N-(2-oxo-2-(piperidin-1-yl)ethyl)acetamide

### Example 138

### N-Methyl-2-((1-methyl-5-((((4-(trifluoromethyl)benzyl)oxy)imino)methyl)-1H-indol-2-yl)oxy)-N-(2-oxo-2-(piperidin-1-yl)ethyl)acetamide

### Example 139

### 2-((1-Methyl-5-((((4-(trifluoromethyl)benzyl)oxy)imino)methyl)-1H-indol-2-yl)oxy)-N-(2-oxo-2-(piperidin-1-yl)ethyl)acetamide

### Example 140

### 2-((5-((((4-Fluorobenzyl)oxy)imino)methyl)-1-methyl-1H-indol-2-yl)oxy)-N-(2-oxo-2-(piperidin-1-yl)ethyl)acetamide

### Example 141

### N-Methyl-2-((1-methyl-4-(1-(((3-phenylallyl)oxy)imino)propyl)-1H-pyrrol-2-yl)oxy)-N-(2-oxo-2-(piperidin-1-yl)ethyl)acetamide

### Example 142

### N-Benzyl-2-((1-methyl-4-(1-(((3-phenylallyl)oxy)imino)propyl)-1H-pyrrol-2-yl)oxy)acetamide

### Example 143

### 2-((1-Methyl-4-(1-(((3-phenylallyl)oxy)imino)propyl)-1H-pyrrol-2-yl)oxy)-N-(4-(trifluoromethyl)benzyl)acetamide

### Example 144

### 2-((1-Methyl-4-(1-((3-phenylpropoxy)imino)propyl)-1H-pyrrol-2-yl)oxy)-N-(4-(trifluoromethyl)benzyl)acetamide

### Example 145

### 2-((1-Methyl-4-(1-((3-phenylpropoxy)imino)propyl)-1H-pyrrol-2-yl)oxy)-N-(naphthalen-2-ylmethyl)acetamide

### Example 146

### 2-(4-(1-((Dibenzo[b,d]thiophen-3-ylmethoxy)imino)-3-methoxypropyl)phenoxy)-N-methyl-N-(2-oxo-2-(piperidin-1-yl)ethyl)acetamide

### Example 147

### 2-(4-(1-((Benzo[b]thiophen-6-ylmethoxy)imino)-3-methoxypropyl)phenoxy)-N-methyl-N-(2-oxo-2-(piperidin-1-yl)ethyl)acetamide

### Example 148

### 2-(4-(1-((Benzyloxy)imino)-3-methoxypropyl)phenoxy)-N-methyl-N-(2-oxo-2-(piperidin-1-yl)ethyl)acetamide

### Example 149

### 2-((5-(1-((Benzyloxy)imino)-3-methoxypropyl)pyridin-2-yl)oxy)-N-methyl-N-(2-oxo-2-(piperidin-1-yl)ethyl)acetamide

### Example 150

### 2-((4-(1-((Benzyloxy)imino)propyl)-1-methyl-1H-pyrrol-2-yl)oxy)-N-methyl-N-(2-oxo-2-(piperidin-1-yl)ethyl)acetamide

### Example 151

### 2-(4-(1-((2-(6-Azaspiro[2.5]octan-6-yl)ethoxy)imino)ethyl)phenoxy)-N-((3,4-dihydro-2H-pyran-4-yl)methyl)acetamide

### Example 152

### 2-(4-(1-((Benzyloxy)imino)ethyl)phenoxy)-N-((3,4-dihydro-2H-pyran-4-yl)methyl)acetamide

### Example 153

### N-((3,4-Dihydro-2H-pyran-4-yl)methyl)-2-(4-(1-(((4-(trifluoromethyl)benzyl)oxy)imino)ethyl)phenoxy)acetamide

### Example 154

### N-((3,4-Dihydro-2H-pyran-4-yl)methyl)-2-(4-(1-(((6-(trifluoromethyl)pyridin-3-yl)methoxy)imino)ethyl)phenoxy)acetamide

### Example 155

### N-((3,4-Dihydro-2H-pyran-4-yl)methyl)-2-(4-(2-hydroxy-1-(((6-(trifluoromethyl)pyridin-3-yl)methoxy)imino)ethyl)phenoxy)acetamide

### Example 156

### N-((3,4-Dihydro-2H-pyran-4-yl)methyl)-2-(4-(2-methoxy-1-(((6-(trifluoromethyl)pyridin-3-yl)methoxy)imino)ethyl)phenoxy)acetamide

### Example 157

### N-((3,4-Dihydro-2H-pyran-4-yl)methyl)-2-(4-(1-(((6-(trifluoromethyl)pyridin-3-yl)methoxy)imino)butyl)phenoxy)acetamide

### Example 158

### N-((3,6-Dihydro-2H-pyran-4-yl)methyl)-2-(4-(1-(((4-(trifluoromethyl)benzyl)oxy)imino)ethyl)phenoxy)acetamide

### Example 159

### N-((3,6-Dihydro-2H-pyran-4-yl)methyl)-2-(4-(1-((naphthalen-2-ylmethoxy)imino)ethyl)phenoxy)acetamide

### Example 160

### N-((3,6-Dihydro-2H-pyran-4-yl)methyl)-2-((5-(1-((naphthalen-2-ylmethoxy)imino)ethyl)pyridin-2-yl)oxy)acetamide

### Example 161

### N-((3,6-Dihydro-2H-pyran-4-yl)methyl)-2-((1-methyl-4-(1-((naphthalen-2-ylmethoxy)imino)ethyl)-1H-pyrrol-2-yl)oxy)acetamide

### Example 162

### N-((3,6-Dihydro-2H-pyran-4-yl)methyl)-2-((1-methyl-4-(1-((quinolin-7-ylmethoxy)imino)ethyl)-1H-pyrrol-2-yl)oxy)acetamide

### Example 163

### 2-((4-(1-((2-(6-Azaspiro[2.5]octan-6-yl)ethoxy)imino)ethyl)-1-methyl-1H-pyrrol-2-yl)oxy)-N-((3,6-dihydro-2H-pyran-4-yl)methyl)acetamide

### Example 164

### 2-(4-(1-((2-(6-Azaspiro[2.5]octan-6-yl)ethoxy)imino)ethyl)phenoxy)-N-((3,6-dihydro-2H-pyran-4-yl)methyl)acetamide

### Example 165

### N-((3,6-Dihydro-2H-pyran-4-yl)methyl)-2-((4-(((4-(trifluoromethyl)benzyl)oxy)imino)chroman-7-yl)oxy)acetamide

### Example 166

### N-((3,6-Dihydro-2H-pyran-4-yl)methyl)-2-(4-(1-(((4-(trifluoromethyl)benzyl)oxy)imino)propyl)phenoxy)acetamide

### Example 167

### N-((3,6-Dihydro-2H-pyran-4-yl)methyl)-2-(4-(1-(((4-fluorobenzyl)oxy)imino)propyl)phenoxy)acetamide

### Example 168

### N-((3,6-Dihydro-2H-pyran-4-yl)methyl)-2-(4-(1-(((4-fluorobenzyl)oxy)imino)propyl)phenoxy)ethanethioamide

### Example 169

### N-((3,6-Dihydro-2H-pyran-4-yl)methyl)-2-(4-(1-(((4-methoxybenzyl)oxy)imino)propyl)phenoxy)ethanethioamide

### Example 170

### N-((3,6-Dihydro-2H-pyran-4-yl)methyl)-2-(4-(1-(((4-methoxybenzyl)oxy)imino)-2-phenylethyl)phenoxy)ethanethioamide

### Example 171

### N-((3,6-Dihydro-2H-pyran-4-yl)methyl)-2-(4-(1-(((4-methoxybenzyl)oxy)imino)-2-phenylethyl)phenoxy)acetamide

### Example 172

### 2-(4-(1-((Bicyclo[2.2.2]octan-2-yloxy)imino)ethyl)phenoxy)-1-(piperidin-1-yl)ethanone

### Example 173

### 2-(4-(1-((Bicyclo[2.2.2]octan-2-yloxy)imino)-2-phenylethyl)phenoxy)-1-(piperidin-1-yl)ethanone

### Example 174

### 2-(4-(1-((Bicyclo[2.2.2]octan-2-yloxy)imino)-2-cyclohexylethyl)phenoxy)-1-(piperidin-1-yl)ethanone

### Example 175

### 2-(4-(1-((Bicyclo[2.2.2]octan-2-yloxy)imino)-2-morpholinoethyl)phenoxy)-1-(piperidin-1-yl)ethanone

### Example 176

### 2-((5-(1-((Bicyclo[2.2.2]octan-2-yloxy)imino)-2-morpholinoethyl)pyridin-2-yl)oxy)-1-(piperidin-1-yl)ethanone

### Example 177

### 2-((8-((Bicyclo[2.2.2]octan-2-yloxy)imino)-5,6,7,8-tetrahydroisoquinolin-3-yl)oxy)-1-(piperidin-1-yl)ethanone

### Example 178

### 2-(4-(1-((3-(Bicyclo[2.2.2]octan-2-yloxy)propoxy)imino)propyl)phenoxy)-N-(cyclohexylmethyl)acetamide

### Example 179

### 2-(4-(1-((3-(Bicyclo[2.2.2]octan-2-yloxy)propoxy)imino)propyl)phenoxy)-1-morpholinoethanone

### Example 180

### 2-(4-(1-((3-(Bicyclo[2.2.2]octan-2-yloxy)propoxy)imino)propyl)phenoxy)-N-phenylacetamide

### Example 181

### 2-(4-(1-((3-(Bicyclo[2.2.2]octan-2-yloxy)propoxy)imino)propyl)phenoxy)-N-(2-cyanophenyl)acetamide

### Example 182

### 2-(4-(1-((Bicyclo[2.2.2]octan-2-yloxy)imino)propyl)phenoxy)-N-(2-cyanophenyl)acetamide

### Example 183

### 2-(4-(1-((Bicyclo[2.2.2]octan-2-yloxy)imino)propyl)phenoxy)-N-((1-methylpiperidin-4-yl)methyl)acetamide

### Example 184

### 2-(4-(1-((Bicyclo[2.2.2]octan-2-yloxy)imino)ethyl)phenoxy)-N-((1-methylpiperidin-4-yl)methyl)acetamide

### Demonstration of In Vitro Potency of Compounds

The PCSK9-LDLR in vitro Binding Assay is a quantitative solid phase binding assay between PCSK9 and recombinant LDLR. Plates were pre-coated with a recombinant LDLR-AB domain, which binds PCSK9. Test compound at different concentration was added to the PCSK9 and added to LDLR immobilized on the wells. The amount of bound PCSK9 is measured by binding it with biotinylated anti-His-tag monoclonal antibody, followed by binding with horseradish peroxidase conjugated streptavidin substrate. The color was quantified by ELISA reader at 450 nM which reflects the relative amount of PCSK9 that binds to LDLR in presence and absence of the inhibitor.

| | Concentration | % Inhibition |
|---|---|---|
| Example No. | (μM) | PCSK9 |
| 1 | 10 | 50 |
| | 100 | 51 |
| 3 | 10 | 32 |
| | 100 | 38 |
| 4 | 10 | 31 |
| | 100 | 62 |
| 6 | 10 | 49 |
| 8 | 10 | 50 |
| | 100 | 52 |
| 9 | 10 | 53 |
| | 100 | 33 |
| 10 | 10 | 21 |
| | 100 | 53 |
| 11 | 10 | 26 |
| | 100 | 57 |
| 13 | 10 | 52 |
| | 100 | 54 |
| 15 | 10 | 32 |
| | 100 | 64 |
| 20 | 10 | 20 |
| | 100 | 54 |
| 22 | 10 | 35 |
| | 100 | 60 |
| 25 | 10 | 27 |
| | 100 | 40 |
| 26 | 10 | 32 |
| | 100 | 44 |
| 34 | 10 | 25 |
| | 100 | 46 |
| 35 | 10 | 26 |
| | 100 | 42 |
| 36 | 10 | 22 |
| | 100 | 40 |
| 40 | 10 | 24 |
| | 100 | 50 |
| 46 | 10 | 27 |
| | 100 | 59 |
| 50 | 10 | 22 |
| | 100 | 42 |
| 54 | 10 | 40 |
| | 100 | 62 |
| 56 | 10 | 63 |
| | 100 | 66 |
| 57 | 10 | 50 |
| | 100 | 59 |
| 58 | 10 | 50 |
| | 100 | 53 |
| 59 | 10 | 49 |
| | 100 | 79 |
| 60 | 10 | 61 |
| | 100 | 91 |
| 62 | 10 | 57 |
| | 100 | 73 |
| 63 | 10 | 48 |
| | 100 | 58 |

The compounds of the present invention are suitable for the treatment and/or mitigation of obesity, hyperlipidaemia, hypercholesteremia, hypertension, atherosclerotic disease events, vascular restenosis, diabetes and many other related conditions in humans and animals. The pharmaceutical compositions containing the compounds of the present invention optionally with another suitable pharmaceutical agent can comprise of one or more pharmaceutically acceptable excipients as is known in the art. The formulation can be prepared by suitable techniques well known. The formulation may be in the form of a tablet, capsule, caplet, satches etc. which are well known to a skilled person. The doses may vary depending on the disease, gravity of the disease, risk profile of the user etc.

1. Compound of the general formula (I), including its isomers and tautomeric forms wherein, 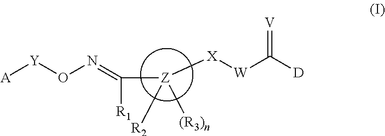
‘A’ represents an optionally substituted single or fused or spirocyclic or bridgeheaded group selected from aryl, heteroaryl or cycloalkyl groups; ‘Y’ represents either a bond or substituted or unsubstituted linear or branched (C₁-C₆)alkyl, (C₂-C₆)alkenyl groups or the groups represented by ‘-U(CH₂)ₘ—’ wherein U represents O, NR₈; ‘m’ represents integers from 2 to 4, and R₈ represents H, substituted or unsubstituted linear or branched (C₁-C₆)alkyl; ‘V’ represents O or S; ‘Z’ represents an optionally substituted single or fused group selected from aryl, or heterocyclyl groups; ‘X’ represents either a bond, or O; ‘W’ represents substituted or unsubstituted linear or branched (C₁-C₆)alkyl, (C₂-C₆)alkenyl groups; R₁ represents hydrogen, optionally substituted (C₁-C₆)alkyl, (C₃-C₆)cycloalkyl, alkoxyalkyl, hydroxyalkyl, aminoalkyl, aryl, heterocyclyl, aralkyl, heterocyclylalkyl groups; R₂ represents hydrogen, or the groups selected from (C₁-C₆)alkyl, aryl, heterocyclyl, aralkyl, heterocyclylalkyl, (C₁-C₆)alkoxy, hydroxyalkyl, thio(C₁-C₆)alkyl, amino, aminoalkyl, alkylamino, halogen, hydroxyl, ester, formyl, acyl, haloalkyl each of which may be optionally substituted; Alternatively R₁ and R₂ wherever possible, together forms a 4 to 7 membered saturated or partially saturated ring containing from 0-2 additional heteroatoms selected from the group consisting of N, O, and S(O)ₒ wherein ‘o’ represents integer from 0 to 2; R₃ at each occurrence independently represents hydrogen, halogen, (C₁-C₃)alkyl, halo(C₁-C₃)alkyl, (C₁-C₃)alkoxy, hydroxyl, esters, formyl, acyl, thio(C₁-C₃)alkyl, sulfenyl derivatives, sulfonyl derivatives; ‘D’ represents —NR₄R₅ or the group —N(R₆)(CH₂)ₚCONR₄R₅;
R₄, R₅, R₆ at each occurrence independently represents
i) H, (C₁-C₆) linear or branched alkyl, (C₁-C₆) linear or branched alkenyl, (C₁-C₆) linear or branched alkynyl, hydroxy, (C₁-C₆) alkoxy, (C₁-C₆) alkenoxy, hydroxy(C₁-C₆)alkyl, alkoxyalkyl, haloalkyl, (C₃-C₆) cycloalkyl, thio(C₁-C₆)alkyl, (C₁-C₆)alkylthio, halo, oxo, imino, nitro, saturated heterocyclyl, optionally substituted amino, amino(C₁-C₆)alkyl, alkylamino, cyano, formyl, acyl, haloalkoxy, aryl, aryloxy, aralkyl, aralkoxy, heterocyclylalkyl, heterocycloxy, heterocyclylalkoxy groups or the groups selected from carboxylic acid and its derivatives such as esters and amides, alkylsulfonyl, alkylsulfonylamino, alkylsulfonyloxy, each of which may be optionally substituted with a provision that when ‘D’ represents —NR₄R₅ and either of R₄ or R₅ is H the other one does not represent 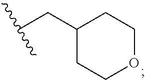 [SMILES: *CC1CCOCC1]
or either of R₄ or R₅ is optionally substituted aryl or heteroaryl group when X represents O; or
ii) R₄ and R₅ wherever possible, together forms optionally substituted 4 to 7 membered saturated or partially saturated ring containing from 0-2 additional heteroatoms selected from the group consisting of N, O, and S(O)ₒ; wherein ‘o’ is as defined earlier; or,
iii) R₄ and R₅ together with N atom may form optionally substituted spirocyclic group containing from 0-2 additional heteroatoms selected from the group consisting of N, O, and S(O)ₒ; wherein ‘o’ is as defined earlier;
‘p’ represents integers from 0 to 5; ‘n’ represents integers from 0-3.

2. The compound as claimed in claim 1 wherein A′ is selected from optionally substituted aryl or heteroaryl groups.

3. The compound as claimed in claim 2 wherein when ‘A’ represents and aryl group, aryl group is selected from substituted or unsubstituted monocyclic or bicyclic aromatic groups wherein substituents selected from halogen, (C₁-C₆)alkyl, CF₃, OCF₃, OCH₃, SO₂CH₃ phenyl.

4. The compound as claimed in claim 3 wherein the aryl group is an optionally substituted phenyl group, the substituents are selected from (C₁-C₆)alkyl, CF₃, OCF₃, OCH₃.

5. The compound as claimed in claim 2 wherein when ‘A’ represents a heteroaryl group, the heteroaryl group is selected from single or fused mono, bi or tricyclic aromatic groups containing one or more hetero atoms selected from O, N or S which optionally substituted with substituents selected from halogen, (C₁-C₆)alkyl, CF₃, OCF₃, OCH₃, SO₂CH₃ phenyl group further optionally substituted with CH₃, CF₃, OCH₃ or halogen.

6. The compound as claimed in claim 5 wherein the heteroaryl group is selected from pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzofuranyl, benzothienyl, indolinyl, indolyl, azaindolyl, azaindolinyl, pyrazolopyrimidinyl, azaquinazolinyl, pyridofuranyl, pyridothienyl, thienopyrimidyl, quinolinyl, pyrimidinyl, pyrazolyl, quinazolinyl, pyridazinyl, triazinyl, benzimidazolyl, benzotriazolyl, phthalazynil, naphthylidinyl, purinyl, carbazolyl, phenothiazinyl, phenoxazinyl, benzoxazolyl, benzothiazolyl, thiazepinyl, oxazolidinyl, thiazolidinyl, benzopyranyl, benzopyranonyl, benzodihydrofuranyl, benzodihydrothienyl, pyrazolopyrimidonyl, azaquinazolinoyl, thienopyrimidonyl, quinazolonyl, pyrimidonyl, benzoxazinyl, benzoxazinonyl, benzothiazinyl, benzothiazinonyl, thieno piperidinyl groups.

7. The compound as claimed in claim 1 wherein ‘Z’ is selected from optionally substituted aryl or groups wherein the substituents are selected from halogen, (C₁-C₆)alkyl, CF₃, CF₃, OCH₃, SO₂CH₃.

8. The compound as claimed in claim 7 wherein when ‘Z’ represents an aryl group, the aryl group is selected from substituted or unsubstituted monocyclic or bicyclic aromatic groups.

9. The compound as claimed in claim 8 wherein the aryl group is an optionally substituted phenyl group.

10. The compound as claimed in claim 7 wherein when ‘Z’ represents a heterocyclyl group, the heterocyclyl group is selected from single or fused mono or bi cyclic aromatic or non-aromatic groups containing one or more hetero atoms selected from O, N or S.

11. The compound as claimed in claim 10 wherein ‘Z’ represents a heteroaromatic group selected from pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzofuranyl, benzothienyl, indolinyl, indolyl, azaindolyl, azaindolinyl, pyrazolopyrimidinyl, azaquinazolinyl, pyridofuranyl, pyridothienyl, thienopyrimidyl, quinolinyl, pyrimidinyl, pyrazolyl, quinazolinyl, pyridazinyl, triazinyl, benzimidazolyl, benzotriazolyl, phthalazynil, naphthylidinyl, purinyl, carbazolyl, phenothiazinyl, phenoxazinyl, benzoxazolyl, benzothiazolyl groups.

12. The compound as claimed in claim 1 selected from
2-(2-Methyl-4-(2-phenyl-1-(((4-(trifluoromethyl)benzyl)oxy)imino)ethyl)phenoxy)-1-morpholinoethanone;
1-Morpholino-2-(4-(2-phenyl-1-(((4-(trifluoromethyl)benzyl)oxy)imino)ethyl)phenoxy)ethanone;
2-(2-Methyl-4-(2-phenyl-1-(((4-(trifluoromethyl)benzyl)oxy)imino)ethyl)phenoxy)-1-(4-methylpiperazin-1-yl)ethanone;
1-(4-Methylpiperazin-1-yl)-2-(4-(2-phenyl-1-(((4-(trifluoromethyl)benzyl)oxy)imino)ethyl)phenoxy)ethanone;
2-(2-Methyl-4-(2-phenyl-1-(((4-(trifluoromethyl)benzyl)oxy)imino)ethyl)phenoxy)-N-(2-morpholino-2-oxoethyl)acetamide;
N-(2-Morpholino-2-oxoethyl)-2-(4-(2-phenyl-1-(((4-(trifluoromethyl)benzyl)oxy)imino)ethyl)phenoxy)acetamide;
N-Hydroxy-2-(2-methyl-4-(2-phenyl-1-(((4-(trifluoromethyl)benzyl)oxy)imino)ethyl)phenoxy)acetamide;
tert-Butyl4-((2-(4-(2-phenyl-1-(((4-(trifluoromethyl)benzyl)oxy)imino)ethyl)phenoxy)acetamido)oxy)piperidine-1-carboxylate;
N-Morpholino-2-(4-(2-phenyl-1-(((4-(trifluoromethyl)benzyl)oxy)imino)ethyl)phenoxy)acetamide;
2-(2-Methyl-4-(2-(thiophen-3-yl)-1-(((4-(trifluoromethyl)benzyl)oxy)imino)ethyl)phenoxy)-1-morpholinoethanone;
1-Morpholino-2-(4-(1-(((4-(trifluoromethyl)benzyl)oxy)imino)ethyl)phenoxy)ethanone;
1-(4-Methylpiperazin-1-yl)-2-(4-(1-(((4-(trifluoromethyl)benzyl)oxy)imino)ethyl)phenoxy)ethanone;
1-Morpholino-2-(4-(1-(((4-(trifluoromethyl)benzyl)oxy)imino)propyl)phenoxy)ethanone
1-(4-Methylpiperazin-1-yl)-2-(4-(1-(((4-(trifluoromethyl)benzyl)oxy)imino)propyl)phenoxy)ethanone;
1-(Piperidin-1-yl)-2-(4-(1-(((4-(trifluoromethyl)benzyl)oxy)imino)propyl)phenoxy)ethanone;
N-Cyclopentyl-2-(4-(1-(((4-(trifluoromethyl)benzyl)oxy)imino)propyl)phenoxy)acetamide;
N-Cyclopropyl-2-(4-(1-(((4-(trifluoromethyl)benzyl)oxy)imino)propyl)phenoxy)acetamide;
4-(2-(4-(1-(((4-(Trifluoromethyl)benzyl)oxy)imino)propyl)phenoxy)acetyl)piperazin-2-one;
N-(2-Hydroxyethyl)-N-phenyl-2-(4-(1-(((4-(trifluoromethyl)benzyl)oxy)imino)propyl)phenoxy)acetamide;
N-Morpholino-2-(4-(1-(((4-(trifluoromethyl)benzyl)oxy)imino)propyl)phenoxy)acetamide;
1-(4-Hydroxypiperidin-1-yl)-2-(4-(1-(((4-(trifluoromethyl)benzyl)oxy)imino)propyl)phenoxy)ethanone;
N-(1,3-Dimethyl-1H-pyrazol-5-yl)-2-(4-(1-(((4-(trifluoromethyl)benzyl)oxy)imino)propyl)phenoxy)acetamide;
2-(2-Methyl-4-(1-(((4-(trifluoromethyl)benzyl)oxy)imino)butyl)phenoxy)-1-morpholinoethanone;
2-(2-Methyl-4-(1-(((4-(trifluoromethyl)benzyl)oxy)imino)butyl)phenoxy)-1-(4-methylpiperazin-1-yl)ethanone;
2-(4-(1-((Benzyloxy)imino)-2-phenylethyl)phenoxy)-1-morpholinoethanone;
2-(2-Methyl-4-(1-(((4-methylbenzyl)oxy)imino)-2-phenylethyl)phenoxy)-1-morpholinoethanone;
2-(4-(1-(((4-Methylbenzyl)oxy)imino)-2-phenylethyl)phenoxy)-1-morpholinoethanone;
2-(4-(1-(((4-Fluorobenzyl)oxy)imino)-2-phenylethyl)-2-methylphenoxy)-1-morpholinoethanone;
2-(4-(1-(((4-Fluorobenzyl)oxy)imino)-2-phenylethyl)phenoxy)-1-morpholinoethanone;
2-(4-(1-(((4-Fluorobenzyl)oxy)imino)-2-phenylethyl)phenoxy)-1-(4-methylpiperazin-1-yl)ethanone;
2-(4-(1-(((4-Fluorobenzyl)oxy)imino)-2-phenylethyl)phenoxy)-N-(2-morpholino-2-oxoethyl)acetamide;
2-(4-(1-(((4-Chlorobenzyl)oxy)imino)-2-phenylethyl)-2-methylphenoxy)-1-morpholinoethanone;
2-(4-(1-(((4-Chlorobenzyl)oxy)imino)-2-phenylethyl)phenoxy)-1-morpholinoethanone;
2-(2-Methyl-4-(2-phenyl-1-(((4-(trifluoromethoxy)benzyl)oxy)imino)ethyl)phenoxy)-1-morpholinoethanone;
1-Morpholino-2-(4-(2-phenyl-1-(((4-(trifluoromethoxy)benzyl)oxy)imino)ethyl)phenoxy)ethanone;
2-(4-(1-(((4-Methoxybenzyl)oxy)imino)-2-phenylethyl)-2-methylphenoxy)-1-morpholinoethanone;
2-(4-(1-(((4-Methoxybenzyl)oxy)imino)-2-phenylethyl)phenoxy)-1-morpholinoethanone;
2-(4-(1-(((4-Methoxybenzyl)oxy)imino)-2-phenylethyl)-2-methylphenoxy)-1-(4-methylpiperazin-1-yl)ethanone;
2-(4-(1-(((4-Methoxybenzyl)oxy)imino)-2-phenylethyl)phenoxy)-1-(4-methylpiperazin-1-yl)ethanone;
2-(4-(1-(((4-Methoxybenzyl)oxy)imino)-2-phenylethyl)-2-methylphenoxy)-1-(piperidin-1-yl)ethanone;
2-(4-(1-(((4-Methoxybenzyl)oxy)imino)-2-phenylethyl)phenoxy)-1-(piperidin-1-yl)ethanone;
2-(4-(1-(((4-(Methylsulfonyl)benzyl)oxy)imino)propyl)phenoxy)-1-morpholinoethanone;
1-(4-Methylpiperazin-1-yl)-2-(4-(1-(((4-(methylsulfonyl)benzyl)oxy)imino)propyl)phenoxy)ethanone;
1-Morpholino-2-(4-(2-phenyl-1-((pyridin-2-ylmethoxy)imino)ethyl)phenoxy)ethanone;
2-(4-(1-((2-(1H-Indol-1-yl)ethoxy)imino)propyl)phenoxy)-1-morpholinoethanone;
2-(4-(1-((2-(1H-Indol-1-yl)ethoxy)imino)propyl)phenoxy)-1-(4-methylpiperazin-1-yl)ethanone;
3-Methyl-2-((((1-(4-(2-morpholino-2-oxoethoxy)phenyl)propylidene)amino)oxy)methyl)quinazolin-4(3H)-one;
2-(4-(1-(((5-Ethylpyrimidin-2-yl)oxy)imino)propyl)phenoxy)-1-morpholinoethanone;
2-(4-(1-(((5-Ethylpyrimidin-2-yl)oxy)imino)propyl)phenoxy)-1-(4-methylpiperazin-1-yl)ethanone;
2-(4-(1-(((5-Methyl-2-phenyloxazol-4-yl)methoxy)imino)propyl)phenoxy)-1-(4-methylpiperazin-1-yl)ethanone;
2-(4-(1-(((5-Methyl-2-phenyloxazol-4-yl)methoxy)imino)propyl)phenoxy)-1-morpholinoethanone;
2-(4-(1-(((3-(tert-Butyl)-1-(p-tolyl)-1H-pyrazol-5-yl)methoxy)imino)propyl)phenoxy)-1-morpholinoethanone;
2-(4-(1-(((3-(tert-Butyl)-1-(p-tolyl)-1H-pyrazol-5-yl)methoxy)imino)propyl)phenoxy)-1-(4-methylpiperazin-1-yl)ethanone;
1-(Piperidin-1-yl)-2-((5-(((4-(trifluoromethyl)benzyl)oxy)imino)-5,6,7,8-tetrahydronaphthalen-2-yl)oxy)ethanone;
1-Morpholino-2-((5-(((4-(trifluoromethyl)benzyl)oxy)imino)-5,6,7,8-tetrahydronaphthalen-2-yl)oxy)ethanone;
1-(4-Methylpiperazin-1-yl)-2-((5-(((4-(trifluoromethyl)benzyl)oxy)imino)-5,6,7,8-tetrahydronaphthalen-2-yl)oxy)ethanone;
N-Morpholino-2-((5-(((4-(trifluoromethyl)benzyl)oxy)imino)-5,6,7,8-tetrahydronaphthalen-2-yl)oxy)acetamide;
N-(Piperidin-1-yl)-2-((5-(((4-(trifluoromethyl)benzyl)oxy)imino)-5,6,7,8-tetrahydronaphthalen-2-yl)oxy)acetamide;
N-(5-Chlorobenzo[d]oxazol-2-yl)-2-((5-(((4-(trifluoromethyl)benzyl)oxy)imino)-5,6,7,8-tetrahydronaphthalen-2-yl)oxy)acetamide;
N-(4-Methylpyrimidin-2-yl)-2-((5-(((4-(trifluoromethyl)benzyl)oxy)imino)-5,6,7,8-tetrahydronaphthalen-2-yl)oxy)acetamide;
N-(1,3-Dimethyl-1H-pyrazol-5-yl)-2-((5-(((4-(trifluoromethyl)benzyl)oxy)imino)-5,6,7,8-tetrahydronaphthalen-2-yl)oxy)acetamide;
tert-Butyl-4-((2-((5-(((4-(trifluoromethyl)benzyl)oxy)imino)-5,6,7,8-tetrahydronaphthalen-2-yl)oxy)acetamido)oxy)piperidine-1-carboxylate;
N-(2-Isocyanophenyl)-2-((5-(((4-(trifluoromethyl)benzyl)oxy)imino)-5,6,7,8-tetrahydronaphthalen-2-yl)oxy)acetamide;
2-((4-((Naphthalen-2-ylmethoxy)imino)chroman-7-yl)oxy)-1-(piperidin-1-yl)ethanone;
2-((4-((Benzyloxy)imino)chroman-7-yl)oxy)-1-(piperidin-1-yl)ethanone;
2-((4-(((4-Fluorobenzyl)oxy)imino)chroman-7-yl)oxy)-1-(piperidin-1-yl)ethanone;
1-(Piperidin-1-yl)-2-((4-(((4-(trifluoromethyl)benzyl)oxy)imino)chroman-7-yl)oxy)ethanone;
2-((4-(((4-Methoxybenzyl)oxy)imino)chroman-7-yl)oxy)-1-(piperidin-1-yl)ethanone;
2-((4-(((4-Methoxybenzyl)oxy)imino)chroman-7-yl)oxy)-1-morpholinoethanone;
2-((4-(((4-Methoxybenzyl)oxy)imino)chroman-7-yl)oxy)-1-(4-methylpiperazin-1-yl)ethanone;
8-(2-((5-(((4-(Trifluoromethyl)benzyl)oxy)imino)-5,6,7,8-tetrahydronaphthalen-2-yl)oxy)acetyl)-1-oxa-3,8-diazaspiro[4.5]decan-2-one;
8-(2-((4-(((4-(Trifluoromethyl)benzyl)oxy)imino)chroman-7-yl)oxy)acetyl)-1-oxa-3,8-diazaspiro[4.5]decan-2-one;
8-(2-((4-(((4-Methoxybenzyl)oxy)imino)chroman-7-yl)oxy)acetyl)-1-oxa-3,8-diazaspiro[4.5]decan-2-one;
8-(2-((4-((Naphthalen-2-ylmethoxy)imino)chroman-7-yl)oxy)acetyl)-1-oxa-3,8-diazaspiro[4.5]decan-2-one;
8-(2-((4-(((6-(Trifluoromethyl)pyridin-3-yl)methoxy)imino)chroman-7-yl)oxy)acetyl)-1-oxa-3,8-diazaspiro[4.5]decan-2-one;
8-(2-((4-(((1-Methyl-1H-indol-6-yl)methoxy)imino)chroman-7-yl)oxy)acetyl)-1-oxa-3,8-diazaspiro[4.5]decan-2-one;
8-(2-(4-(1-(((4-Methoxybenzyl)oxy)imino)propyl)phenoxy)acetyl)-1-oxa-3,8-diazaspiro[4.5]decan-2-one;
8-(2-(4-(1-(((4-(Trifluoromethyl)benzyl)oxy)imino)propyl)phenoxy)acetyl)-1-oxa-3,8-diazaspiro[4.5]decan-2-one;
8-(2-(4-(1-(((4-(Trifluoromethyl)benzyl)oxy)imino)ethyl)phenoxy)acetyl)-1-oxa-3,8-diazaspiro[4.5]decan-2-one;
8-(2-(4-(1-(((4-Methoxybenzyl)oxy)imino)ethyl)phenoxy)acetyl)-1-oxa-3,8-diazaspiro[4.5]decan-2-one;
8-(2-(4-(1-((4-Methoxyphenoxy)imino)ethyl)phenoxy)acetyl)-1-oxa-3,8-diazaspiro[4.5]decan-2-one;
8-(2-(4-(1-((4-(Trifluoromethyl)phenoxy)imino)ethyl)phenoxy)acetyl)-1-oxa-3,8-diazaspiro[4.5]decan-2-one;
1-(6-Azaspiro[2.5]octan-6-yl)-2-(4-(1(((4(trifluoromethyl)benzyl)oxy)imino)ethyl)phenoxy)ethanone;
1-(6-Azaspiro[2.5]octan-6-yl)-2-(4-(1-(((4-(trifluoromethyl)benzyl)oxy)imino)propyl)phenoxy)ethanone;
2-(4-(2-Phenyl-1-(((4-(trifluoromethyl)benzyl)oxy)imino)ethyl)phenoxy)-1-(6-azaspiro[2.5]octan-6-yl)ethanone;
2-(4-(1-(((4-Methoxybenzyl)oxy)imino)propyl)phenoxy)-1-(6-azaspiro[2.5]octan-6-yl)ethanone;
2-(4-(1-(((4-Fluorobenzyl)oxy)imino)propyl)phenoxy)-1-(6-azaspiro[2.5]octan-6-yl)ethanone;
2-(4-(1-((3-(6-Azaspiro[2.5]octan-6-yl)propoxy)imino)propyl)phenoxy)-1-(piperidin-1-yl)ethanone;
2-(4-(1-((2-(6-Azaspiro[2.5]octan-6-yl)ethoxy)imino)propyl)phenoxy)-1-(piperidin-1-yl)ethanone;
2-(4-(1-((2-(Methyl(phenyl)amino)ethoxy)imino)propyl)phenoxy)-1-(piperidin-1-yl)ethanone;
2-(4-(1-((2-((4-Fluorophenyl)(methyl)amino)ethoxy)imino)propyl)phenoxy)-1-(piperidin-1-yl)ethanone;
2-(4-(1-((2-((4-Fluorophenyl)(methyl)amino)ethoxy)imino)propyl)phenoxy)-1-morpholinoethanone;
2-(4-(1-((2-((4-Fluorophenyl)(methyl)amino)ethoxy)imino)propyl)phenoxy)-1-thiomorpholinoethanone;
2-(4-(1-((Naphthalen-2-ylmethoxy)imino)propyl)phenoxy)-1-(6-azaspiro[2.5]octan-6-yl)ethanone;
2-(4-(1-((Naphthalen-2-ylmethoxy)imino)pentyl)phenoxy)-1-(6-azaspiro[2.5]octan-6-yl)ethanone;
2-(4-(1-(((3,4-Dimethylbenzyl)oxy)imino)pentyl)phenoxy)-1-(6-azaspiro[2.5]octan-6-yl)ethanone;
2-(4-(1-(((3,4-Dimethylbenzyl)oxy)imino)-3-methoxypropyl)phenoxy)-1-(6-azaspiro[2.5]octan-6-yl)ethanone;
2-(4-(1-((Dibenzo[b,d]thiophen-3-ylmethoxy)imino)-3-methoxypropyl)phenoxy)-1-(6-azaspiro[2.5]octan-6-yl)ethanone;
2-(4-(1-((Dibenzo[b,d]thiophen-3-ylmethoxy)imino)-3-methoxypropyl)phenoxy)-1-(piperidin-1-yl)ethanone;
N-((1-Methylpiperidin-4-yl)oxy)-2-(4-(2-morpholino-1-(((4-(trifluoromethyl)benzyl)oxy)imino)ethyl)phenoxy)ethanethioamide;
2-(4-(1-(((3,5-Bis(trifluoromethyl)benzyl)oxy)imino)-2-morpholinoethyl)phenoxy)-N-((1-methylpiperidin-4-yl)oxy)ethanethioamide;
2-(4-(1-(((3,5-Bis(trifluoromethyl)benzyl)oxy)imino)-2-morpholinoethyl)phenoxy)-N-((1-methylpiperidin-4-yl)oxy)acetamide;
2-(4-(1-(((3,5-Bis(trifluoromethyl)benzyl)oxy)imino)-2-(piperidin-1-yl)ethyl)phenoxy)-2-methyl-N-((1-methylpiperidin-4-yl)methyl)propanamide;
2-(4-(2-Methoxy-1-(((4-(trifluoromethyl)benzyl)oxy)imino)ethyl)phenoxy)-N-((1-methylpiperidin-4-yl)methyl)ethanethioamide;
2-(4-(2-Methoxy-1-(((4-(trifluoromethyl)benzyl)oxy)imino)ethyl)phenoxy)-1-(piperidin-1-yl)ethanethione;
2-(4-(1-(([1,1′-Biphenyl]-4-ylmethoxy)imino)-2-methoxyethyl)phenoxy)-1-(piperidin-1-yl)ethanethione;
2-(4-(2-Methoxy-1-(((4′-(trifluoromethyl)-[1,1′-biphenyl]-4-yl)methoxy)imino)ethyl)phenoxy)-1-(piperidin-1-yl)ethanethione;
2-(4-(2-Methoxy-1-(((4′-(trifluoromethyl)-[1,1′-biphenyl]-4-yl)methoxy)imino)ethyl)phenoxy)-1-(4-methylpiperazin-1-yl)ethanethione;
N-(Cyclohexylmethyl)-3-(4-(2-phenyl-1-(((4-(trifluoromethyl)benzyl)oxy)imino)ethyl)phenyl) propanethioamide;
N-((1-Methylpiperidin-4-yl)methyl)-2-(4-(2-phenyl-1-(((4-(trifluoromethyl)benzyl)oxy)imino)ethyl)phenoxy)ethanethioamide;
2-(4-(2-Cyclohexyl-1-(((4-(trifluoromethyl)benzyl)oxy)imino)ethyl)phenoxy)-N-((1-methylpiperidin-4-yl)methyl)ethanethioamide;
N-((1-Methylpiperidin-4-yl)methyl)-2-(4-(2-morpholino-1-(((4-(trifluoromethyl)benzyl)oxy)imino)ethyl)phenoxy)ethanethioamide;
1-(4-Allylpiperazin-1-yl)-2-(4-(1-((pyridin-4-ylmethoxy)imino)propyl)phenoxy)ethanone;
1-(4-Allylpiperazin-1-yl)-2,2-difluoro-2-(4-(1-((pyridin-4-ylmethoxy)imino)propyl)phenoxy)ethanone;
N-(Cyclohexylmethyl)-2,2-difluoro-N-methyl-2-(4-(1-((pyridin-4-ylmethoxy)imino)propyl)phenoxy)acetamide;
N-(Cyclohexylmethyl)-2,2-difluoro-2-(4-(1-((pyridin-4-ylmethoxy)imino)propyl)phenoxy)acetamide;
2-(4-(1-((Benzyloxy)imino)propyl)phenoxy)-N-(cyclohexylmethyl)-2,2-difluoroacetamide;
N-(Cyclohexylmethyl)-2,2-difluoro-2-(4-(1-(((4-(trifluoromethyl)benzyl)oxy)imino)propyl)phenoxy)acetamide;
N-(Cyclohexylmethyl)-2-(4-(1-(((4-(trifluoromethyl)benzyl)oxy)imino)propyl)phenoxy)ethanethioamide;
2-(4-(1-(((1,2,3,4-Tetrahydronaphthalen-1-yl)oxy)imino)propyl)phenoxy)-1-(4-(3-(trifluoromethyl)phenyl)piperazin-1-yl)ethanone;
2-(4-(1-((Cyclohexyloxy)imino)propyl)phenoxy)-1-(4-(3-(trifluoromethyl)phenyl)piperazin-1-yl)ethanone;
2-(4-(1-((Benzyloxy)imino)propyl)phenoxy)-1-(4-(3-(trifluoromethyl)phenyl)piperazin-1-yl)ethanone;
2-(4-(1-(((2-Fluorobenzyl)oxy)imino)propyl)phenoxy)-1-(4-(3-(trifluoromethyl)phenyl)piperazin-1-yl)ethanone;
2-(4-(1-((Pyridin-4-ylmethoxy)imino)propyl)phenoxy)-1-(4-(3-(trifluoromethyl)phenyl)piperazin-1-yl)ethanone;
1-(4-Phenylpiperazin-1-yl)-2-(4-(1-((pyridin-4-ylmethoxy)imino)propyl)phenoxy)ethanone;
8-(2-(4-(1-(((1,2,3,4-Tetrahydronaphthalen-1-yl)oxy)imino)propyl)phenoxy)acetyl)-1-oxa-3,8-diazaspiro[4.5]decan-2-one;
1-(Piperidin-1-yl)-2-(4-(1-(((1,2,3,4-tetrahydronaphthalen-1-yl)oxy)imino)propyl)phenoxy)ethanone;
1-(4-(2-Hydroxyethyl)piperazin-1-yl)-2-(4-(1-(((1,2,3,4-tetrahydronaphthalen-1-yl)oxy)imino)propyl)phenoxy)ethanone;
2-Hydroxy-1-(4-(2-(4-(1-(((1,2,3,4-tetrahydronaphthalen-1-yl)oxy)imino)propyl)phenoxy)acetyl)piperazin-1-yl)ethanone;
2-Hydroxy-1-(4-(2-(4-(1-(((2-methylbenzyl)oxy)imino)propyl)phenoxy)acetyl)piperazin-1-yl)ethanone;
2-Hydroxy-1-(4-(2-(4-(1-(((2-(trifluoromethyl)benzyl)oxy)imino) propyl)phenoxy)acetyl)piperazin-1-yl)ethanone;
2-(4-(1-(((5-Fluoro-2-(trifluoromethyl)benzyl)oxy)imino)propyl)phenoxy)-1-(4-(2-hydroxyacetyl)piperazin-1-yl)ethanone;
N-Methyl-2-((1-methyl-5-((phenoxyimino)methyl)-1H-indol-2-yl)oxy)-N-(2-oxo-2-(piperidin-1-yl)ethyl)acetamide;
2-((5-(((Benzyloxy)imino)methyl)-1-methyl-1H-indol-2-yl)oxy)-N-methyl-N-(2-oxo-2-(piperidin-1-yl)ethyl)acetamide;
N-Methyl-2-((1-methyl-5-((((4-(trifluoromethyl)benzyl)oxy)imino)methyl)-1H-indol-2-yl)oxy)-N-(2-oxo-2-(piperidin-1-yl)ethyl)acetamide;
2-((1-Methyl-5-((((4-(trifluoromethyl)benzyl)oxy)imino)methyl)-1H-indol-2-yl)oxy)-N-(2-oxo-2-(piperidin-1-yl)ethyl)acetamide;
2-((5-((((4-Fluorobenzyl)oxy)imino)methyl)-1-methyl-1H-indol-2-yl)oxy)-N-(2-oxo-2-(piperidin-1-yl)ethyl)acetamide;
N-Methyl-2-((1-methyl-4-(1-(((3-phenylallyl)oxy)imino)propyl)-1H-pyrrol-2-yl)oxy)-N-(2-oxo-2-(piperidin-1-yl)ethyl)acetamide;
N-Benzyl-2-((1-methyl-4-(1-(((3-phenylallyl)oxy)imino)propyl)-1H-pyrrol-2-yl)oxy)acetamide;
2-((1-Methyl-4-(1-(((3-phenylallyl)oxy)imino)propyl)-1H-pyrrol-2-yl)oxy)-N-(4-(trifluoromethyl)benzyl)acetamide;
2-((1-Methyl-4-(1-((3-phenylpropoxy)imino)propyl)-1H-pyrrol-2-yl)oxy)-N-(4-(trifluoromethyl)benzyl)acetamide;
2-((1-Methyl-4-(1-((3-phenylpropoxy)imino)propyl)-1H-pyrrol-2-yl)oxy)-N-(naphthalen-2-ylmethyl)acetamide;
2-(4-(1-((Dibenzo[b,d]thiophen-3-ylmethoxy)imino)-3-methoxypropyl)phenoxy)-N-methyl-N-(2-oxo-2-(piperidin-1-yl)ethyl)acetamide;
2-(4-(1-((Benzo[b]thiophen-6-ylmethoxy)imino)-3-methoxypropyl)phenoxy)-N-methyl-N-(2-oxo-2-(piperidin-1-yl)ethyl)acetamide;
2-(4-(1-((Benzyloxy)imino)-3-methoxypropyl)phenoxy)-N-methyl-N-(2-oxo-2-(piperidin-1-yl)ethyl)acetamide;
2-((5-(1-((Benzyloxy)imino)-3-methoxypropyl)pyridin-2-yl)oxy)-N-methyl-N-(2-oxo-2-(piperidin-1-yl)ethyl)acetamide;
2-((4-(1-((Benzyloxy)imino)propyl)-1-methyl-1H-pyrrol-2-yl)oxy)-N-methyl-N-(2-oxo-2-(piperidin-1-yl)ethyl)acetamide;
2-(4-(1-((2-(6-Azaspiro[2.5]octan-6-yl)ethoxy)imino)ethyl)phenoxy)-N-((3,4-dihydro-2H-pyran-4-yl)methyl)acetamide;
2-(4-(1-((Benzyloxy)imino)ethyl)phenoxy)-N-((3,4-dihydro-2H-pyran-4-yl)methyl)acetamide;
N-((3,4-Dihydro-2H-pyran-4-yl)methyl)-2-(4-(1-(((4-(trifluoromethyl)benzyl)oxy)imino)ethyl)phenoxy)acetamide;
N-((3,4-Dihydro-2H-pyran-4-yl)methyl)-2-(4-(1-(((6-(trifluoromethyl)pyridin-3-yl)methoxy)imino)ethyl)phenoxy)acetamide;
N-((3,4-Dihydro-2H-pyran-4-yl)methyl)-2-(4-(2-hydroxy-1-(((6-(trifluoromethyl)pyridin-3-yl)methoxy)imino)ethyl)phenoxy)acetamide;
N-((3,4-dihydro-2H-pyran-4-yl)methyl)-2-(4-(2-methoxy-1-(((6-(trifluoromethyl)pyridin-3-yl)methoxy)imino)ethyl)phenoxy)acetamide;
N-((3,4-Dihydro-2H-pyran-4-yl)methyl)-2-(4-(1-(((6-(trifluoromethyl)pyridin-3-yl)methoxy)imino)butyl)phenoxy)acetamide;
N-((3,6-Dihydro-2H-pyran-4-yl)methyl)-2-(4-(1-(((4-(trifluoromethyl)benzyl)oxy)imino)ethyl)phenoxy)acetamide;
N-((3,6-Dihydro-2H-pyran-4-yl)methyl)-2-(4-(1-((naphthalen-2-ylmethoxy)imino)ethyl)phenoxy)acetamide;
N-((3,6-Dihydro-2H-pyran-4-yl)methyl)-2-((5-(1-((naphthalen-2-ylmethoxy)imino)ethyl)pyridin-2-yl)oxy)acetamide;
N-((3,6-Dihydro-2H-pyran-4-yl)methyl)-2-((1-methyl-4-(1-((naphthalen-2-ylmethoxy)imino)ethyl)-1H-pyrrol-2-yl)oxy)acetamide;
N-((3,6-Dihydro-2H-pyran-4-yl)methyl)-2-((1-methyl-4-(1-((quinolin-7-ylmethoxy)imino)ethyl)-1H-pyrrol-2-yl)oxy)acetamide;
2-((4-(1-((2-(6-Azaspiro[2.5]octan-6-yl)ethoxy)imino)ethyl)-1-methyl-1H-pyrrol-2-yl)oxy)-N-((3,6-dihydro-2H-pyran-4-yl)methyl)acetamide;
2-(4-(1-((2-(6-Azaspiro[2.5]octan-6-yl)ethoxy)imino)ethyl)phenoxy)-N-((3,6-dihydro-2H-pyran-4-yl)methyl)acetamide;
N-((3,6-Dihydro-2H-pyran-4-yl)methyl)-2-((4-(((4-(trifluoromethyl)benzyl)oxy)imino)chroman-7-yl)oxy)acetamide;
N-((3,6-Dihydro-2H-pyran-4-yl)methyl)-2-(4-(1-(((4-(trifluoromethyl)benzyl)oxy)imino)propyl)phenoxy)acetamide;
N-((3,6-Dihydro-2H-pyran-4-yl)methyl)-2-(4-(1-(((4-fluorobenzyl)oxy)imino)propyl)phenoxy)acetamide;
N-((3,6-Dihydro-2H-pyran-4-yl)methyl)-2-(4-(1-(((4-fluorobenzyl)oxy)imino)propyl)phenoxy)ethanethioamide;
N-((3,6-Dihydro-2H-pyran-4-yl)methyl)-2-(4-(1-(((4-methoxybenzyl)oxy)imino)propyl)phenoxy)ethanethioamide;
N-((3,6-Dihydro-2H-pyran-4-yl)methyl)-2-(4-(1-(((4-methoxybenzyl)oxy)imino)-2-phenylethyl)phenoxy)ethanethioamide;
N-((3,6-Dihydro-2H-pyran-4-yl)methyl)-2-(4-(1-(((4-methoxybenzyl)oxy)imino)-2-phenylethyl)phenoxy)acetamide;
2-(4-(1-((Bicyclo[2.2.2]octan-2-yloxy)imino)ethyl)phenoxy)-1-(piperidin-1-yl)ethanone;
2-(4-(1-((Bicyclo[2.2.2]octan-2-yloxy)imino)-2-phenylethyl)phenoxy)-1-(piperidin-1-yl)ethanone;
2-(4-(1-((Bicyclo[2.2.2]octan-2-yloxy)imino)-2-cyclohexylethyl)phenoxy)-1-(piperidin-1-yl)ethanone;
2-(4-(1-((Bicyclo[2.2.2]octan-2-yloxy)imino)-2-morpholinoethyl)phenoxy)-1-(piperidin-1-yl)ethanone;
2-((5-(1-((Bicyclo[2.2.2]octan-2-yloxy)imino)-2-morpholinoethyl)pyridin-2-yl)oxy)-1-(piperidin-1-yl)ethanone;
2-((8-((Bicyclo[2.2.2]octan-2-yloxy)imino)-5,6,7,8-tetrahydroisoquinolin-3-yl)oxy)-1-(piperidin-1-yl)ethanone;
2-(4-(1-((3-(Bicyclo[2.2.2]octan-2-yloxy)propoxy)imino)propyl)phenoxy)-N-(cyclohexylmethyl)acetamide;
2-(4-(1-((3-(Bicyclo[2.2.2]octan-2-yloxy)propoxy)imino)propyl)phenoxy)-1-morpholinoethanone;
2-(4-(1-((3-(Bicyclo[2.2.2]octan-2-yloxy)propoxy)imino)propyl)phenoxy)-N-phenylacetamide;
2-(4-(1-((3-(Bicyclo[2.2.2]octan-2-yloxy)propoxy)imino)propyl)phenoxy)-N-(2-cyanophenyl)acetamide;
2-(4-(1-((Bicyclo[2.2.2]octan-2-yloxy)imino)propyl)phenoxy)-N-(2-cyanophenyl)acetamide;
2-(4-(1-((Bicyclo[2.2.2]octan-2-yloxy)imino)propyl)phenoxy)-N-((1-methylpiperidin-4-yl)methyl)acetamide;
2-(4-(1-((Bicyclo[2.2.2]octan-2-yloxy)imino)ethyl)phenoxy)-N-((1-methylpiperidin-4-yl)methyl)acetamide;

13. The compounds as claimed in claim 12 suitably formulated into a suitable pharmaceutical composition.

14. A method to treat hyperlipidemia or dyslipidemia comprising administering to a subject in need thereof an effective amount of a compound of formula (I) as claimed in claim 1 so as to treat hyperlipidemia or dyslipidemia.

15. (canceled)

16. A pharmaceutical composition comprising a compound of formula (I) as claimed in claim 1 in combination with one or more pharmaceutically active agents selected from group comprising insulin, insulin derivatives and mimetics, insulin secretagogues, insulin sensitizers, biguanide agents, alpha-glucosidase inhibitors, insulinotropic sulfonylurea receptor ligands, meglitinides, GLP-1 (glucagon like peptide-1), GLP-1 analogs, DPPIV (dipeptidyl peptidase IV) inhibitors, GPR-119 activators, sodium-dependent glucose co-transporter (SGLT2) inhibitors, PPAR modulators, non-glitazone type PPAR delta agonist, HMG-CoA reductase inhibitors, cholesterol-lowering drugs, rennin inhibitors, anti-thrombotic and anti-platelet agents and anti-obesity agents or pharmaceutically acceptable salts thereof.

17. A method to treat hyperlipidemia or dyslipidemia comprising administering to a subject in need thereof an effective amount of the pharmaceutical composition as claimed in claim 16 so as to treat dyslipidemia or hyperlipidemia.
